(19) United States
(12) Patent Application Publication
(10) Pub. No.: US 2003/0158209 A1
(43) Pub. Date: Aug. 21, 2003

(54) Ligands of melanocortin receptors and compositions and methods related thereto

(75) Inventors: Brian P. Dyck, San Diego, CA (US); Val Goodfellow, Encinitas, CA (US); Teresa Phillips, San Diego, CA (US); Jessica Parker, San Diego, CA (US); Xiaohu Zhang, San Diego, CA (US); Chen Chen, San Diego, CA (US); Joe Anh Tran, San Marcos, CA (US); Joseph Pontillo, San Diego, CA (US); Fabio C. Tucci, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences Inc., San Diego, CA

(21) Appl. No.: 10/268,923

(22) Filed: Oct. 9, 2002

Related U.S. Application Data
(60) Provisional application No. 60/328,295, filed on Oct. 9, 2001.
(60) Provisional application No. 60/366,745, filed on Mar. 22, 2002.

(51) Int. Cl. A61K 31/496; A61K 31/495; C 07D 4 3/02; C07D 241/04

(52) U.S. Cl. 514/253.01; 514/255.01; 544/386; 544/360

(57) ABSTRACT

Compounds which function as melanocortin receptor ligands and having utility in the treatment of melanocortin receptor-based disorders. The compounds have the following structure (I):

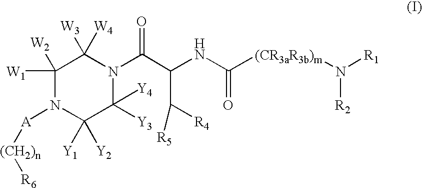

including stereoisomers, prodrugs, and pharmaceutically acceptable salts thereof, wherein A, m, n, R₁, R₂, R₃ₐ, R_{3b}, R₄, R₅, R₆ W₁, W₂, W₃, W₄, Y₁, Y₂, Y₃ and Y₄ are as defined herein. Pharmaceutical compositions containing a compound of structure (I), as well as methods relating to the use thereof, are also disclosed.

### BACKGROUND OF THE INVENTION

1. Field of the Invention

This invention is generally directed to ligands of a melanocortin receptor, as well as to compositions and methods for using such ligands to alter activity of a melanocortin receptor.

2. Description of the Prior Art

Melanocortin (MC) receptors are members of the family of G-protein coupled receptors. To date, five distinct MC receptors (i.e., MC1-R, MC2-R, MC3-R, MC4-R and MC5-R) have been identified in a variety of tissues and these receptors have been shown to mediate a number of physiological processes. Ligands, including peptides and small molecules, have been shown to act as agonists or antagonists at these receptors.

The role of specific MC receptors in physiological processes has been the object of intense study since their discovery and cloning. These receptors are expressed in a variety of tissues including melanocytes, adrenal cortex, brain, gut, placenta, skeletal muscle, lung, spleen, thymus, bone marrow, pituitary, gonads and adipose tissue. A putative role of MC receptors has been shown in melanocytes, stimulatory actions on learning, attention and memory, motor effects, modification of sexual behavior, facilitation of nerve regeneration, anti-inflammatory and antipyretic effects, and the regulation of food intake and body weight.

The pro-opiomelanocortin (POMC) gene product is processed to produce a number of biologically active peptides that are expressed in the pituitary, and two locations in the brain: the arcuate nucleus of the hypothalamus and the solitary tract nucleus of the brain stem. These peptides elicit a range of biological activities. Two POMC peptides, α-melanocyte stimulating hormone (α-MSH) and adrenocorticotropic hormone (ACTH) control melanocyte and adrenocortical function, respectively, in the periphery.

Cloning studies have defined a family of five melanocortin (MC) receptors that respond to POMC peptides (reviewed in Rec. Prog. Hor. Res. 51:287-318, 1996). Each receptor in this family is pharmacologically distinct in its particular response to the POMC peptides α-MSH, γ-MSH and ACTH and to two peptide antagonists. Among the five receptors, MC4-R has the highest affinity for α-MSH. MC4-R differs from the other MC receptors in that it binds both natural melanocortin antagonists, agouti (Nature 371:799-802, 1994) and agouti-related protein (AgRP) (Biochem. Biophys. Res. Commun. 237:629-631, 1997). In contrast, MC 1-R only binds agouti, MC2-R does not bind AgRP, MC3-R only binds AgRP, and MC5-R has only low affinity binding for AgRP (Mol. Endocrinology 13:148-155, 1999).

The expression of specific MC receptors is restricted anatomically. MC 1-R is expressed primarily in melanocytes, while MC2-R is expressed in adrenocortical cells. MC3-R is expressed in brain, placenta and gut, and MC4-R is expressed primarily in the brain where its mRNA can be detected in nuclei that bind α-MSH. MC4-R is notably absent from adrenal cortex, melanocyte and placental tissues. Both MC3-R and MC4-R are expressed in arcuate and paraventricular neurons. MC5-R is expressed in brain, adipose tissues, muscle and exocrine glands.

α-Melanocyte stimulating hormone (α-MSH) is a tridecapeptide whose principal action (i.e., the activation of a set of G-protein coupled melanocortin receptors), results in a range of physiological responses including pigmentation, sebum production and feeding behavior. Cyclized peptide derivatives of α-MSH are potent modulators of these receptors. When administered by intracerebroventricular (i.c.v) injection into fasted animals, peptides exhibiting MCR-4 antagonist activity increase food intake and body weight. Moreover, overexpression of a naturally occurring peptide antagonist, agouti-related peptide (AgRP) has a similar effect on food intake and body weight. The development of small molecule antagonists of the MC4-R would selectively enhance the feeding response. MC4-R antagonists have a unique clinical potential because such compounds would stimulate appetite as well as decrease metabolic rate. Additionally, chronic MC4-R blockade causes an increase in lean body mass as well as fat mass, and the increase in lean body mass is independent of the increase in fat mass. Orally active forms of a small molecule MC4-R antagonist would provide a therapeutic strategy for indications in which cachexia is a symptom.

The MC receptors are also key mediators of steroid production in response to stress (MC2-R), regulation of weight homeostasis (MC4-R), and regulation of hair and skin pigmentation (MC1-R). They may have additional applications in controlling both insulin regulation (MC4-R) and regulation of exocrine gland function (MC5-R) (Cell 91:789-798, 1997); the latter having potential applications in the treatment of disorders such as acne, dry eye syndrome and blepharitis. Melanocortin peptides have also been reported to have anti-inflammatory activity, although the receptor(s) involved in mediating these effects have not yet been determined. Endocrine disorders such as Cushing's disease and congenital adrenal hyperplasia, which are characterized by elevated levels of ACTH, could be effectively treated with ACTH receptor (MC2-R) antagonists. Some evidence suggests that depression, which is characterized by elevated levels of glucocorticoids, may also be responsive to these same compounds. Similarly, elevated glucocorticoids can be an etiological factor in obesity. Synthetic melanocortin receptor agonists have been shown to initiate erections in men (J. Urol. 160:389-393, 1998). An appropriate MC receptor agonist could be an effective treatment for certain sexual disorders.

MC1-R provides an ideal target for developing drugs that alter skin pigmentation. MC1-R expression is localized to melanocytes where it regulates eumelanin pigment synthesis. Two small clinical trials indicate that broad-spectrum melanocortin agonists induce pigmentation with limited side effects. The desired compound would have a short half-life and be topically applied. Applications include skin cancer prevention, UV-free tanning, inhibition of tanning and treatment of pigmentation disorders, such as tyrosinase-positive albinism.

The role of melanocortin receptors in regulation of adiposity signaling and food intake has been recently reviewed (Nature 404:661-669, 2000). Direct experimental evidence for the individual role of MC4 and MC3 receptors in energy homeostasis has not yet been reported due to the lack of potent and specific MC4 and MC3 agonists. Central administration of synthetic, non-selective MC-3R and MC4-R agonists, such as cyclic side-chain-lactam-modified peptide MT-II suppresses food intake in rodents and monkeys, and stimulates energy expenditure resulting in reduced adiposity (Endocrinology 142:2586-2592,2001). Conversely, selective peptide antagonists of the MC4 receptor stimulate food consumption and result in increased body weight, suggesting the main effects of agonist induced inhibition of food consumption are mediated by MC4-R receptor activity. (European .J. Pharmacol. 405:25-32, 2000). Selective small molecule MC4-R antagonists also stimulate food intake in animal models of cachexia.

Genetically modified animals lacking the MC4-R receptor are hyperphagic and obese (Cell 88:131-141, 1997). Humans with defective melanocortin 4 receptors exhibit marked hyperphagia and increased body mass relative to their normal siblings (Nature Genet. 20:111-114, 1998). In addition, studies with mice lacking functional MC-3 receptors suggest that agonist stimulation of this receptor may also play a role in control of energy homeostasis, feeding efficiency, metabolism and bodyweight (Endocrinology 141:3518-3521, 2000). Therefore MC4-R and MC3-R agonists may be useful in the control of obesity and in treatment of related disorders including diabetes.

Due to their important biological role, a number of agonists and antagonists of the MC receptors have been suggested. For example, U.S. Pat. No. 6,054,556 is directed to a family of cyclic heptapeptides which act as antagonists for MC1, MC3, MC4 and MC5 receptors; U.S. Pat. No. 6,127,381 is directed to isoquinoline compounds which act upon MC receptors for controlling cytokine-regulated physiologic processes and pathologies; and published PCT Application No. WO 00/74679 is directed to substituted piperidine compounds that act as selective agonists of MC4-R. Published PCT Application No. WO01/05401 is directed to small peptides that are MC3-R specific agonists.

Accordingly, while significant advances have been made in this field, there is still a need in the art for ligands to the MC receptors and, more specifically, to agonists and/or antagonists to such receptors, particularly small molecules. There is also a need for pharmaceutical compositions containing the same, as well as methods relating to the use thereof to treat conditions associated with the MC receptors. The present invention fulfills these needs, and provides other related advantages.

### BRIEF SUMMARY OF THE INVENTION

In brief, this invention is directed to compounds that function as melanocortin (MC) receptor ligands. In this context, the term “ligand” means a molecule that binds or forms a complex with one or more of the MC receptors. This invention is also directed to compositions containing one or more MC receptor ligands in combination with one or more pharmaceutically acceptable carriers, as well as to methods for treating conditions or disorders associated with MC receptors.

In one embodiment, this invention is directed to MC; receptor ligands which have the following structure (I):

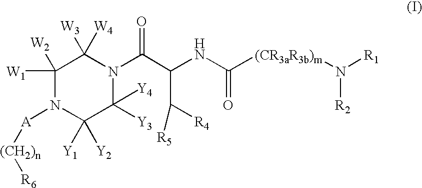

including stereoisomers, prodrugs, and pharmaceutically acceptable salts thereof, wherein A, m, n, R₁, R₂, R₃ₐ, R_{3b}, R₄, R₅, R₆, W₁, W₂, W₃, W₄, Y₁, Y₂, Y₃ and Y₄ are as defined herein.

The MC receptor ligands of this invention have utility over a broad range of therapeutic applications, and may be used to treat disorders or illnesses, including (but not limited to) eating disorders, obesity, inflammation, pain, chronic pain, skin disorders, skin and hair coloration, sexual dysfunction, dry eye, acne, anxiety, depression, and/or Cushing's disease. A representative method of treating such a disorder or illness includes administering an effective amount of a ligand of this invention, preferably in the form of a pharmaceutical composition, to an animal (also referred to herein as a “patient”, including a human) in need thereof. The ligand may be an antagonist or agonist or may stimulate a specific melanocortin receptor while functionally blocking a different melanocortin receptor. Accordingly, in another embodiment, pharmaceutical compositions are disclosed containing one or more ligands of this invention in combination with a pharmaceutically acceptable carrier.

In one embodiment, the MC receptor ligands of this invention are agonists to one or more MC receptors, and are useful in medical conditions where a melanocortin receptor agonist is beneficial. For example, the compounds of this invention may be utilized as MC4-R specific agonists or MC3-R specific agonists. Alternatively, the agonist may have mixed activity on the MC3 and MC4 receptor, and function as an antagonist of one of these receptors.

In this context, the compounds of this invention may be used to treat obesity, erectile and/or sexual dysfunction, or diabetes mellitus. In another embodiment, compounds of this invention may serve as antagonists to either the MC3-R or MC4-R receptor. Such antagonists have beneficial therapeutic effects, especially in the treatment of cachexia or wasting disease associated with cancer, AIDS, failure to thrive syndrome, and diseases associated with aging and senility. In more specific embodiments, the compounds are MC4-R antagonists for treatment of cachexia or wasting disease associated with cancer, AIDs, failure to thrive syndrome, and diseases associated with aging and senility.

These and other aspects of this invention will be apparent upon reference to the following detailed description and attached figures. To that end, certain patent and other documents are cited herein to more specifically set forth various aspects of this invention. Each of these documents is hereby incorporated by reference in its entirety.

### DETAILED DESCRIPTION OF THE INVENTION

As mentioned above, in one embodiment the present invention is generally directed to compounds having the following structure (I):

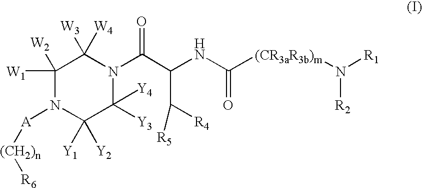

or a stereoisomer, prodrug or pharmaceutically acceptable salt thereof,

wherein:

n is 0, 1, 2, or 3;

m is 1, 2, 3, or 4;

A is alkanediyl optionally substituted with R₇;

R₁ and R₂ are the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl, or —C(═O)R₁₀;

or R₁ and R₂ taken together with the nitrogen atom to which they are attached form heterocycle or substituted heterocycle;

R₃ₐ and R_{3b} are the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;

or R₃ₐ and R_{3b} taken together with the carbon atom to which they are attached form a homocycle, substituted homocycle, heterocycle, or substituted heterocycle;

or R₃ₐ and the carbon atom to which it is attached taken together with one or both of R₁ and R₂ and the nitrogen to which it is attached form heterocycle or substituted heterocycle;

R₄ is aryl, substituted aryl, heteroaryl, or substituted heteroaryl;

R₅ is hydrogen, hydroxy, alkyl, substituted alkyl, aryl, substituted aryl, heterocycle, or substituted heterocycle;

R₆ is cyano, nitro, heterocycle, substituted heterocycle, —NR₈R₉, —C(═O)NR₈R₉, —C(═O)OR₈, —OC(═O)OR₈, —OC(═O)R₈, —OC(═O)NR₈R₉, —NR₈C(═O)OR₈, —NR₈C(═O)R₁₀, —NR₈C(═O)NR₈R₉, —NR₈S(═O)ₚR₁₁, —S(—O)ₚR₁₁, —S(═O)ₚNR₈R₉, —NR₈S(═O)ₚNR₈R₉, or —OR₁₂;

R₇ is alkyl, substituted alkyl, aryl, substituted aryl, arylalkyl, substituted arylalkyl, heterocycle, substituted heterocycle, heterocyclealkyl, substituted heterocyclealkyl, cyano, nitro, —NR₈R₉, —C(═O)NR₈R₉, —C(═O)OR₈, —NR₈C(═O)R₁₀, —NR₈C(═O)NR₈R₉, —NR₈S(═O)ₚR₁₁, —S(O)ₚR₁₁, —NR₈S(═O)ₚNR₈R₉, or —OR₁₂;

R₈ and R₉ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl;

R₁₀, R₁₁ and R₁₂ are the same or different and, at each occurrence, independently hydrogen, halogen, cyano, alkyl, substituted alkyl, aryl, substituted aryl, arylalkyl, substituted arylalkyl, heterocycle, substituted heterocycle, heterocyclealkyl or substituted heterocyclealkyl;

W₁, W₂, W₃, W₄, Y₁, Y₂, Y₃ and Y₄ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, cyano, nitro, —NR₈R₉, —C(═O)NR₈R₉, —C(═O)OR₁₀, —NR₈C(═O)R₁₀, —NR₈C(═O)NR₈R₉, —NR₈S(═O)ₚR₁₁, —S(═O)ₚR₁₁, —NR₈S(═O)ₚNR₈R₉, or —OR₁₂;

or any of one of W₁, W₂, W₃ or W₄ and the carbon to which it is attached together with any one of Y₁, Y₂, Y₃ or Y₄ and the carbon to which it is attached form a bridging heterocycle or substituted heterocycle; and

p is, at each occurrence, 0, 1 or 2.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂cyclohexyl, and the like; while unsaturated cyclic alkyls include cyclopentenyl, cyclohexenyl, —CH₂cyclohexenyl, and the like. Cyclic alkyls are also referred to herein as a “homocycle”, and include bicyclic rings in which a homocycle is fused to a benzene ring. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkanediyl” means a divalent alkyl from which two hydrogen atoms are taken from the same carbon atom or from different carbon atoms, such as —CH₂—, —CH₂CH₂—, —CH₂CH₂CH₂—, —CH(CH₃)CH₂—, -cyclopentane-, -cyclohexane-, -cycloheptane-, and the like.

“Aryl” means an aromatic carbocyclic moiety such as phenyl or naphthyl.

“Arylalkyl” means an alkyl having at least one alkyl hydrogen atom replaced with an aryl moiety, such as benzyl (i.e., —CH₂phenyl), —(CH₂)₂phenyl, —(CH₂)₃phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, oxadiazolyl, benzoxadiazolyl, thiadiazolyl, indazolyl and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

The term “substituted” as used herein means any of the above groups (i.e., alkyl, aryl, arylalkyl, heteroaryl, heteroarylalkyl, heterocycle and heterocyclealkyl) wherein at least one hydrogen atom is replaced with a substituent. In the case of an oxo substituent (“═O”) two hydrogen atoms are replaced. When substituted, “substituents” within the context of this invention include oxo, halogen, hydroxy, cyano, nitro, amino, alkylamino, dialkylamino, alkyl, alkoxy, thioalkyl, haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b} —NRₐSO₂R_{b}, C(═O)Rₐ, —C(═O)ORₐ, —C(═O)NRₐR_{b}, —OC(═O)NRₐR_{b}, ORₐ, —SRₐ, —SORₐ, —S(═O)₂Rₐ, —OS(═O)₂Rₐ, —S(═O)₂ORₐ, —CH₂S(═O)₂Rₐ, —CH₂S(═O)₂NRₐR_{b}, ═NS(═O)₂Rₐ, and —S(═O)₂NRₐR_{b}, wherein Rₐ and R_{b} are the same or different and independently hydrogen, alkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, carbocycle, substituted carbocycle, carbocyclealkyl or substituted carbocyclealkyl.

“Halogen” means fluoro, chloro, bromo and iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

“Alkylamino” and “dialkylamino” mean one or two alkyl moiety attached through a nitrogen bridge (i.e., —N-alkyl) such as methylamino, ethylamino, dimethylamino, diethylamino, and the like.

“Mono- or di(cycloalkyl)methyl” represents a methyl group substituted with one or two cycloalkyl groups, such as cyclopropylmethyl, dicyclopropylmethyl, and the like.

“Alkylcarbonylalkyl” represents an alkyl substituted with a —C(═O)alkyl group.

“Alkylcarbonyloxyalkyl” represents an alkyl substituted with a —C(═O)Oalkyl group or a —OC(═O)alkyl group.

“Mono- or di(alkyl)amino represents an amino substituted with one alkyl or with two alkyls, respectively.

“Alkylamino” and “dialkylamino” mean one or two alkyl moiety attached through a nitrogen bridge (i.e., —N-alkyl) such as methylamino, ethylamino, dimethylamino, diethylamino, and the like.

Depending upon whether the alkanediyl group of moiety “A” is cyclic or noncyclic, representative compounds of the present invention include (but are not limited to) the following structures (Ia) through (Id):

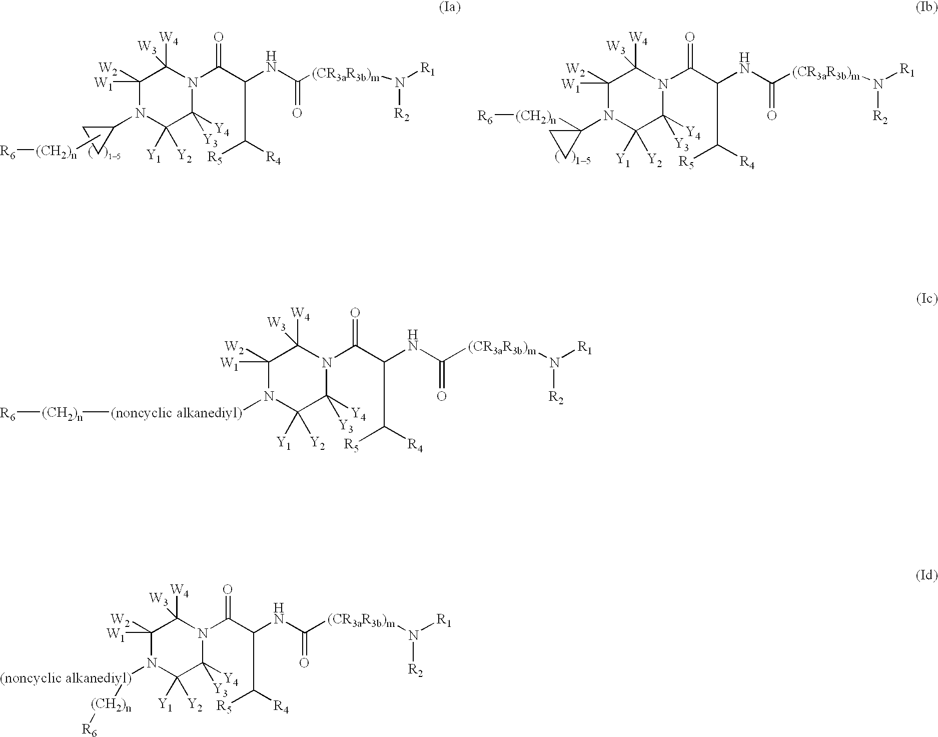

It should be understood that in structure (Ia), the cyclic alkanediyl group includes cyclopropyl, cyclobutyl, cyclopentyl, cyclohexyl and cycloheptyl, wherein the “R₆—(CH₂)ₙ—” group is attached to the carbocyclic ring at any location except the carbon atom that is attached to the nitrogen atom of the piperazine group. This later embodiment being represented by structure (Ib). Similarly, structure (Ic) represents noncyclic alkanediyl groups, wherein the “R₆—(CH₂)ₙ—” group is attached to the alkanediyl group at any location except the carbon atom that is attached to the nitrogen atom of the piperazine group. This later embodiment being represented by structure (Id).

A representative compound where moieties “W₂” and “Y₃” are taken together to form a bridging heterocycle includes (but are not limited to) structure (le), while a representative compound where moieties “R₃ₐ” and “R₁” are taken together to form a heterocycle includes (but is not limited to) structure (If):

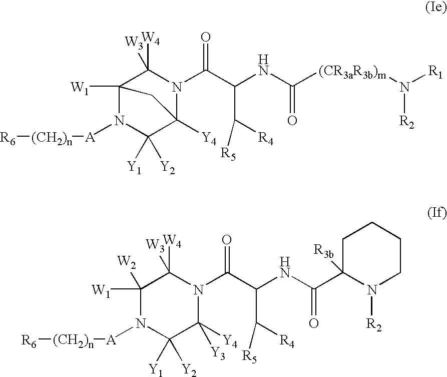

The compounds of the present invention may be prepared by known organic synthesis techniques, including the methods described in more detail in the following Reaction Schemes and Examples. Piperazine subunits of this invention are commercially available, including those having a bridging heterocyle or subsituted heterocyle, are known in the literature or may be synthesized from extensions of known methods. Furthermore, compounds of the present invention may be synthesized by a number of methods, both convergent and sequential, utilizing solution or solid phase chemistry.

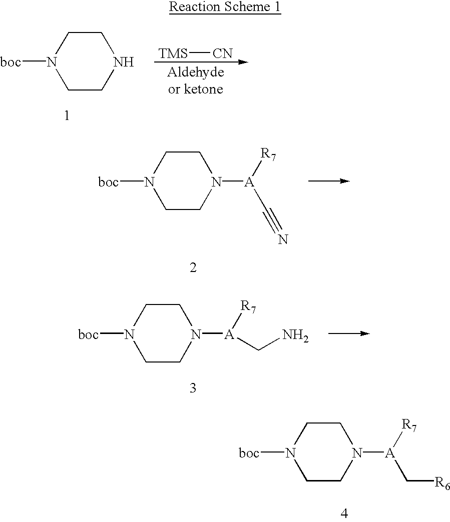

A mono-protected piperazine, here illustrated as N-tert-butyloxycarbonyl-piperazine 1, may be reacted with aldehydes or ketones under the conditions of the Strecker reaction with cyanide or trimethylsilylcyanide to produce a-amino nitrites 2. The procedures are illustrated here with aldehydes but ketones and cyclic ketones may also be used. Reduction of 2 with reagents such as LiAlH₄ produces primary amine intermediate 3 which is versatile for forming a large number of compounds 4, where the nitrogen may be alkylated, acylated, sulfonylated or incorporated into heterocyclic structures.

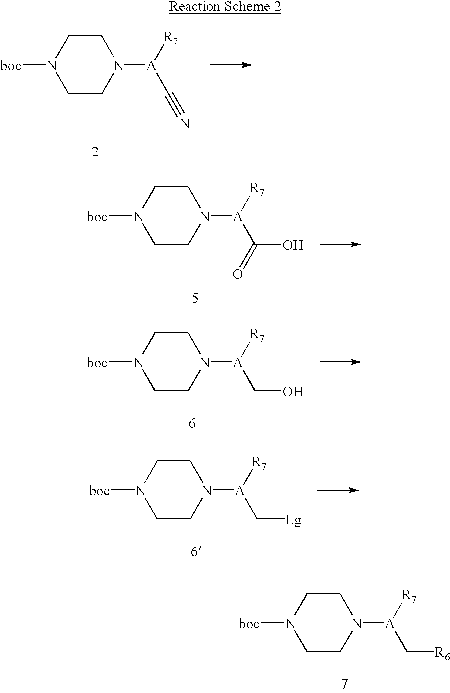

The nitrile 2 may be hydrolyzed, and if necessary protected to provide amino acid 5. LiAlH₄ reduction produces primary alcohol 6. The primary alcohol 6 may be converted to leaving groups such as chlorides, bromides or sulfonyl esters such as mesyl, tosyl, nosyl, triflyl and the like and reacted with nucleophiles. A particularly useful application of this chemistry is to react activated 6′ with heterocyclic molecules to produce compound 7 where R₆ is a triazole or other heterocycle.

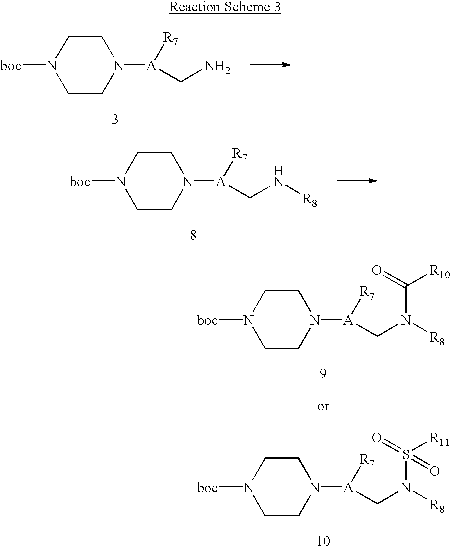

Compound 3 may be reductively alkylated with aldehydes to produce 8 or reacted with sulfonate esters to produce 8, compound 8 in turn may also be acylated or sulfonylated to produce structures such as 9 or 10.

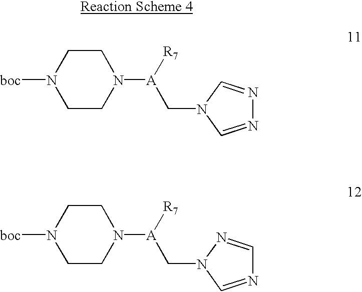

Modification of the displacement conditions (leaving group, solvent, base, phase-transfer conditions) can provide selective regioisomeric modification of heterocycles such as the 1,2,4-triazoles as illustrated. Alternatively reaction of 1,2,4 triazole with acrylonitrile followed by displacement of alkyl mesylates and base elimination of the cyano ethyl group is a directed method for specific alkylation at the 4-position of 1,2,4-triazoles to provide general structures such as 11 (Horvath 1995). A number of similar methods are known in the art for directing alkylation in heterocyclic systems. In addition it is possible to modify alcohol 6 using triphenylphosphine and disubsituted azo derivatives (DEAD, DIAD and the like) to produce derivatized compounds such as 12.

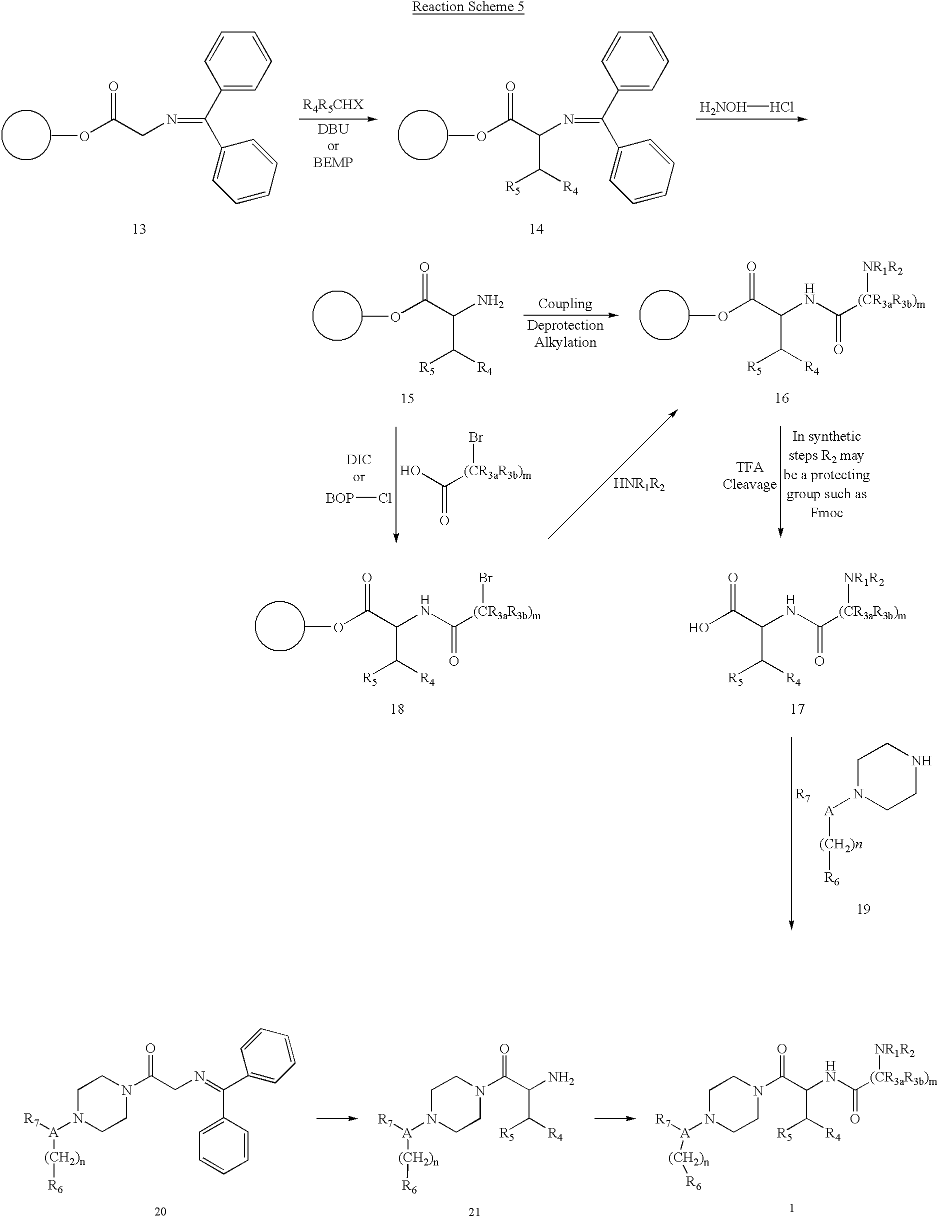

Dipeptide sub-units may be formed by the coupling of protected peptide fragments to a free amine of a piperazine subunit or by stepwise coupling to the piperazine, followed by deprotection, and coupling of individual amino acids by methods well known in the art. A solid state or traditional chemistry methodology may be employed. Novel amino acids in this invention were formed from glycine units 13 which were modified by the reaction with bases such as BEMP or DBU followed by a-carbon alkylation with alkyl halides to form novel α-substituted amino acids 15. Similarly aldol type reactions with 13 and aldehydes and ketones produce novel β-hydroxy amino acids. These methods can be extended to the synthesis of optically active amino acids by use of a chiral auxiliary (O'Donnel 1998). In order make compounds on large scale it is possible to apply the same chemistry to intermediates such as 20 to produce alkylated amino acids such as 21. In addition a variety of methods are well known in the art for producing novel optically active amino acids (Williams, R. M., Synthesis of Optically Active α-Amino Acids, Pergamon Press, Oxford 1989).

Compounds containing N-terminal N-substituted glycines may be synthesized by acylation with substituted bromo acetic acid derivatives to give α-bromo compounds such as 18 followed by displacement with amines in polar aprotic solvents such as DMSO.

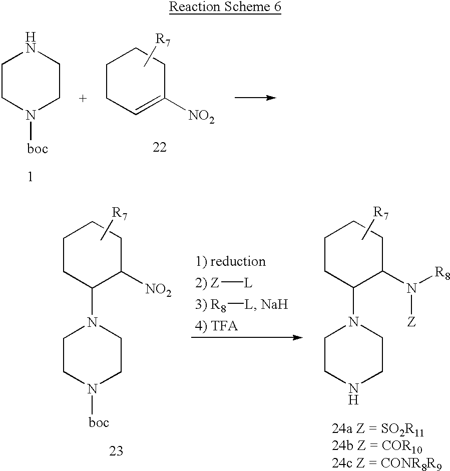

Additional piperazine subunits may be synthesized using the following methodologies or related methods known in the art. Michael addition of piperidine 1 or anions derived from this amine to an appropriate nitro alkene 22 produces nitro substituted-cyclohexyl piperazine 23. Reduction produces a versatile intermediate that may be alkylated, acylated or sulfonylated. In turn these derivatives may be further modified as illustrated.

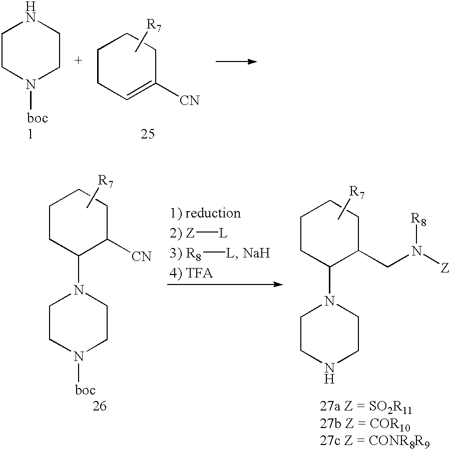

In a similar manner Michael addition of 1 or anions derived from 1 to unsaturated nitrile 25 produces cyanocyclohexyl piperazines 26. Reduction produces amines which may be alkylated, acylated or sulfonylated. These intermediates may also be modified by methods well known in the art to produce structures such as 27.

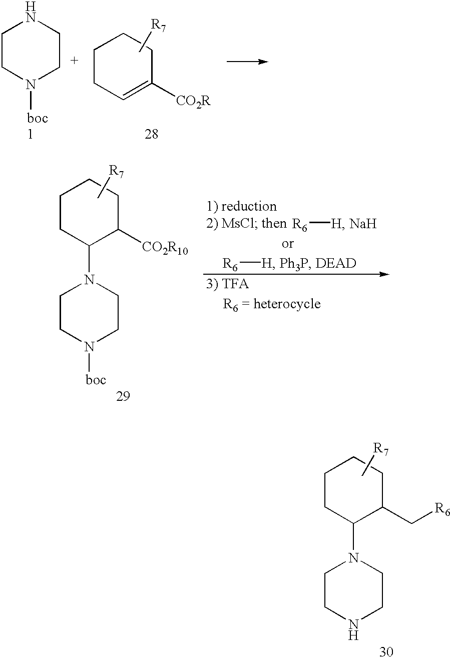

In addition the intermediate amine may be elaborated to produce a variety of heterocyclic substituents of general structure 30.

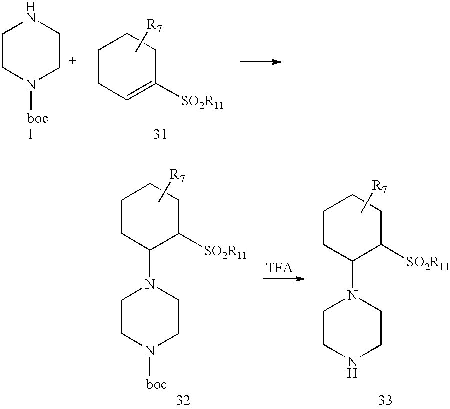

Conjugate addition of piperazines to unsaturated sulfones may also be utilized to produce sulfonyl substituted piperazines 33.

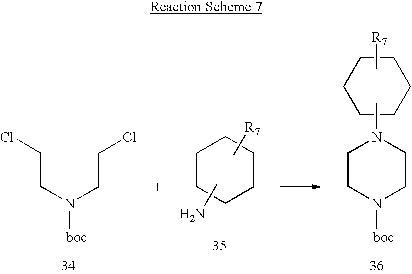

A diverse variety of piperazines suitable for incorporation into structures of general formula 1 are possible using protected and non-protected nitrogen mustards. This process is illustrated for Boc protected mustard reagent 34 reacting with a general cyclic structure 35 to form piperazine subunit of general formula 36. 35 may be cyclic C₃₋₈ or acyclic.

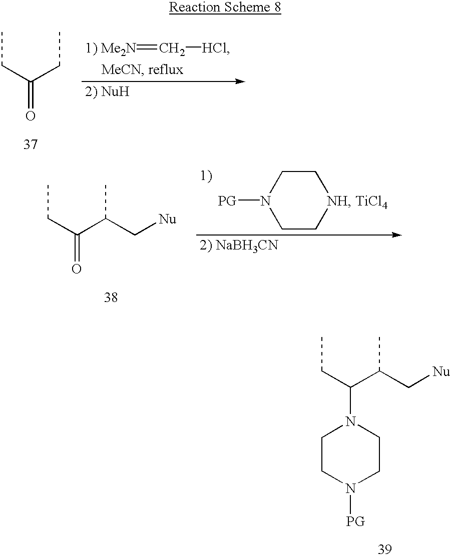

Cyclic or noncyclic ketones 37 in the presence of dimethylammonium chloride and an appropriate nucleophile (NuH) give substituted ketone 38. Reductive alkylation of 38 with a protected piperazine or piperazine analog in the presence of a Lewis acid such as TiCl₄ gives an imine which undergoes hydride reduction to give 39.

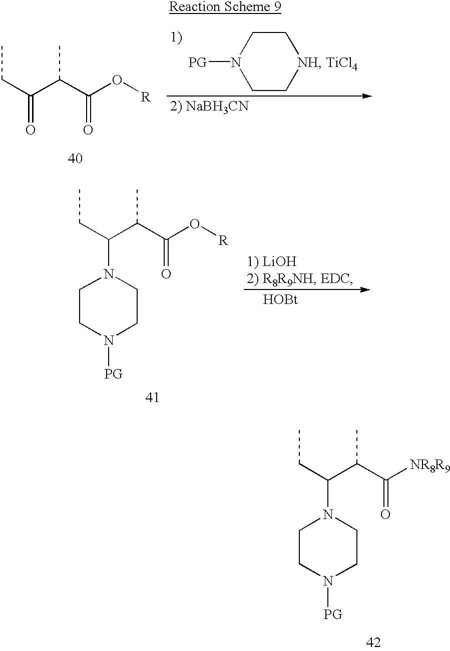

Reductive alkylation of 40 with a protected piperazine or piperazine analog in the presence of a Lewis acid such as TiCl₄ gives an imine which undergoes hydride reduction to give 41. Hydrolysis of the ester followed by amide formation gives 42.

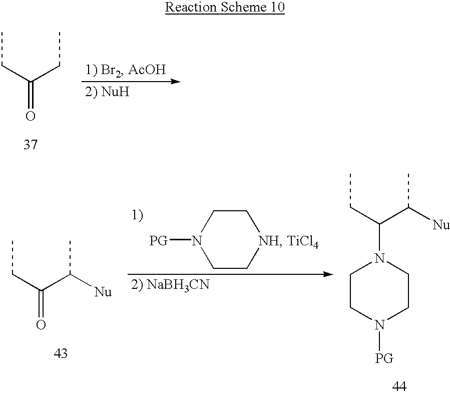

Bromination of 37 using standard conditions such as bromine in acetic acid, is followed by nucleophilic (Nu) displacement to give 43. Reductive alkylation of 43 with a protected piperazine or piperazine analog in the presence of a Lewis acid such as TiCl₄ gives an imine which undergoes hydride reduction to give 44.

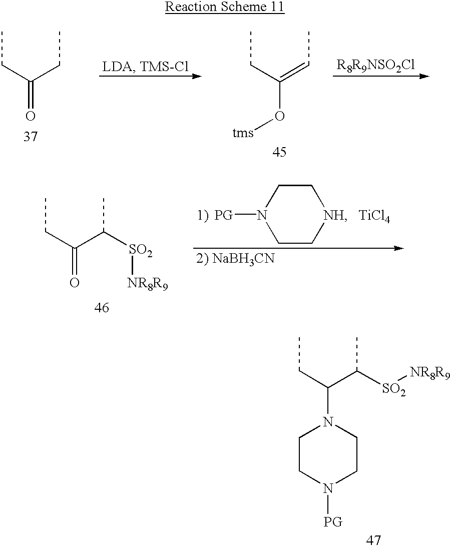

Directed enolization of 37 under conditions such as trimethylsilyl chloride and lithium diisopropylamide gives 45 which which undergoes reaction with a chlorosulfonamide to give α-ketosulfonamide 46. Reductive alkylation of 46 with a protected piperazine or piperazine analog in the presence of a Lewis acid such as TiCl₄ gives an imine which undergoes hydride reduction to give 47.

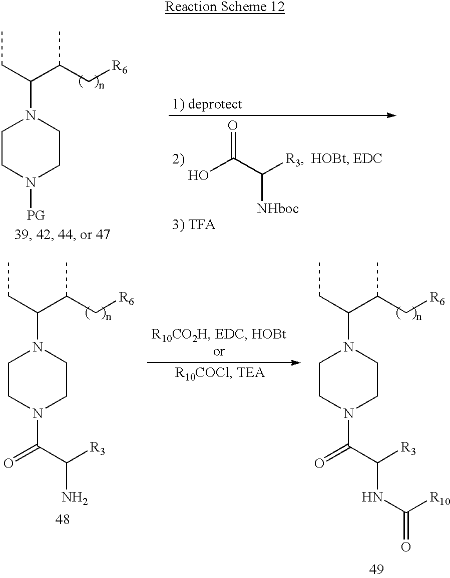

Any of intermediates 39, 42, 44, or 47 are deprotected followed by coupling to a peptide moiety using standard conditions such as 1-hydroxybenzotriazole hydrate (HOBT) and 1-(3-dimethylaminopropyl)-3-ethylcarbodiimide hydrochloride (EDC) to give 48 (following an additional deprotection step using trifluoroacetic acid, if necessary). Addition of a substituted acid via standard peptide coupling conditions or of an acid halide in the presence of a base such as triethylamine gives 49.

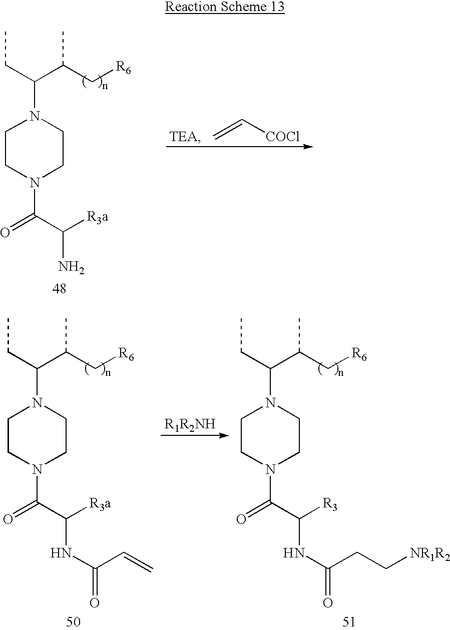

Addition of acryloyl chloride to 48 in the presence of a base such as triethylamine gives acrylamide 50 which may undergo Michael addition with an appropriate amine to give 51.

Representative compounds of this invention include (but are not limited to) the following:

1-{2-(1,2,3,4-Tetrahydro-isoquinoline-3-carboxamido)-3-(4-chlorophenyl)propionyl}-4-{1-[phenylacetamidomethyl]cyclohexyl}piperazine;

1-{2-(2-Amino-3-phenylpropionamido)-3-(4-chlorophenyl)propionyl}-4-{1-[phenylacetamidomethyl]cyclohexyl}piperazine;

1-{2-(2-Amino-indan-2-carboxamido)-3-(4-chlorophenyl)propionyl}-4-{1-[phenylacetamidomethyl]cyclohexyl}piperazine;

1-{2-(2-Amino-indan-2-carboxamido)-3-(4-chlorophenyl)propionyl}-4-{1-[(3-phenylureido)methyl]cyclohexyl}piperazine;

1-{2-(1,2,3,4-Tetrahydro-isoquinoline-3-carboxamido)-3-(4-chlorophenyl)propionyl}-4-{1-[(3-phenylureido)methyl]cyclohexyl}piperazine;

1-{2-(1,2,3,4-Tetrahydro-isoquinoline-3-carboxamido)-3-(4-chlorophenyl)propionyl}-4-{1-[(benzylsulfonamido)methyl]cyclohexyl}piperazine;

1-{2-(1,2,3,4-Tetrahydro-isoquinoline-3-carboxamido)-3-(4-chlorophenyl)propionyl}-4-{1-[(3-phenoxycarbonylamino)methyl]cyclohexyl}piperazine;

1-{2-(1,2,3,4-Tetrahydro-isoquinoline-3-carboxamido)-3-(4-chlorophenyl)propionyl}-4-{1-[(3-phenylthiocarbonylamino)methyl]cyclohexyl}piperazine;

1-{2-(Isoquinoline-3-carboxamido)-3-(4-chlorophenyl)propionyl}-4-{1-[phenylacetamidomethyl]cyclohexyl}piperazine;

1-{2-(2-Amino-1,2,3,4-tetrahydro-naphthalene-2-carboxamido)-3-(4-chlorophenyl)propionyl}-4-{1-[phenylacetanidomethyl]cyclohexyl)}piperazine;

1-{2-(2-Aminopropionamido)-3-(4-chlorophenyl)propionyl}-4-{1-[phenylacetarmidomethyl]cyclohexyl}piperazine;

1-{2-[2-(Methoxycarbonylamino)acetamido)-3-(4-chlorophenyl)propionyl}-4-{1-[phenylacetamidomethyl]cyclohexyl}piperazine;

1-{2-[2-(Methoxycarbonylamino)acetamido)-3-(4-chlorophenyl)propionyl}-4-{1-[(benzylamino)methyl]cyclobexyl}piperazine;

1-{2-[2-(Acetamino)acetamido)-3-(4-chlorophenyl)propionyl}-4-{1-[(benzylamino)methyl]cyclohexyl}piperazine;

1-{2-[2-aminoacetamido)-3-(4-chlorophenyl)propionyl}-4-{1-[(thiazol-2ylmethyl)amino)methyl]cyclohexyl}piperazine;

1-{2-[2-aminoacetamido)-3-(4-chlorophenyl)propionyl}-4-{1-[(pyridin-2-ylamino)methyl]cyclohexyl}piperazine;

1-{2-[2-aminoacetamido)-3-(4-chlorophenyl)propionyl}-4-{1-[(1-imidazol-1-yl)methyl]cyclohexyl}piperazine;

1-{2-[2-aminoacetamido)-3-(4-chlorophenyl)propionyl}-4-{1-[(benzylamino)carbonyl]cyclohexyl}piperazine;

1-{2-[2-aminoacetamido)-3-(4-chlorophenyl)propionyl}-4-{1-[(benzylsulfonamido)methyl]cyclohexyl}piperazine;

1-{2-[2-aminoacetamido)-3-(4-chlorophenyl)propionyl}-4-{1-[(N′-phenyl-guanidino)methyl]cyclohexyl}piperazine;

1-{2-[2-aminoacetamido)-3-(4-chlorophenyl)propionyl}-4-{1-[(1-guanidinocarbonyl)methyl]cyclohexyl}piperazine;

1-{2-[2-aminoacetamido)-3-(4-chlorophenyl)propionyl}-4-{1-[(N-benzyl-guanidinocarbonyl)methyl]cyclohexyl}piperazine;

1-{2-[2-aminoacetamido)-3-(4-chlorophenyl)propionyl}-4-{1-[(N′-benzyl-guanidinocarbonyl)methyl]cyclohexyl}piperazine;

1-{2-[2-aminoacetamido)-3-(4-chlorophenyl)propionyl}-4-{1-[(2-aminoethylaminocarbonyl)methyl]cyclohexyl}piperazine;

1-{2-(3-Aminopropionamido)-3-(2,4-dichlorophenyl)propionyl}-4-{1-[phenylacetamidomethyl]cyclohexyl}piperazine;

1-{2-(3-Aminopropionamido)-3-(2,4-dichlorophenyl)propionyl}-4-{1-[(3-methoxyphenyl)acetamidomethyl]cyclohexyl}piperazine;

1-{2-(3-Aminopropionamido)-3-(2,4-dichlorophenyl)propionyl}-4-{1-[(4-methoxyphenyl)acetamidomethyl]cyclohexyl}piperazine;

1-{2-(3-Aminopropionamido)-3-(2,4-dichlorophenyl)propionyl}-4-{1-[(2-fluorophenyl)acetamidomethyl]cyclohexyl}piperazine;

1-{2-(3-Aminopropionamido)-3-(2,4-dichlorophenyl)propionyl}-4-{1-[(3-fluorophenyl)acetamidomethyl]cyclohexyl}piperazine;

1-{2-(3-Aminopropionamido)-3-(2,4-dichlorophenyl)propionyl}-4-{1-[(4-fluorophenyl)acetamidomethyl]cyclohexyl}piperazine;

1-{2-(3-Aminopropionamido)-3-(2,4-dichlorophenyl)propionyl}-4-{1-[(benzoylamino)methyl]cyclohexyl}piperazine;

1-{2-(3-Aminopropionamido)-3-(2,4-dichlorophenyl)propionyl}-4-{1-[(phenylureido)methyl]cyclohexyl}piperazine;

1-{2-(3-Aminopropionamido)-3-(2,4-dichlorophenyl)propionyl}-4-{1-[(phenylsulfonamido)methyl]cyclohexyl}piperazine;

1-{2-(3-Aminopropionamido)-3-(2,4-dichlorophenyl)propionyl}-4-{1-[(2-fluorobenzylamino)methyl]cyclohexyl}piperazine;

1-{2-(3-Aminopropionamido)-3-(2,4-dichlorophenyl)propionyl}-4-{1-[(benzylamino)methyl]cyclohexyl}piperazine;

1-{2-(3-Aminopropionamido)-3-(2,4-dichlorophenyl)propionyl}-4-{1-[(3-fluorobenzylamino)methyl]cyclohexyl}piperazine;

1-{2-(3-Aminopropionamido)-3-(2,4-dichlorophenyl)propionyl}-4-{1-[(2-methoxybenzylamino)methyl]cyclohexyl}piperazine;

1-{2-(3-Aminopropionamido)-3-(2,4-dichlorophenyl)propionyl}-4-{1-[(2-trifluoromethylbenzylamino)methyl]cyclohexyl}piperazine;

1-{2-(3-Aminopropionamido)-3-(2,4-dichlorophenyl)propionyl}-4-{1-[(2-hydroxylethylamino)methyl]cyclohexyl}piperazine;

1-{2-(3-Aminopropionamido)-3-(2,4-dichlorophenyl)propionyl}-4-{1-[(2-methoxylethylamino)methyl]cyclohexyl}piperazine;

1-{2-(3-Aminopropionamido)-3-(2,4-dichlorophenyl)propionyl}-4-{1-[(1,1,1-trifluoroethylamino)methyl]cyclohexyl}piperazine;

1-{2-(3-Aminopropionamido)-3-(2,4-dichlorophenyl)propionyl}-4-{1-[(phenethylamino)methyl]cyclohexyl}piperazine;

1-{2-(3-Aminopropionamido)-3-(2,4-dichlorophenyl)propionyl}-4-{1-[(2-fluorophenethylamino)methyl]cyclohexyl}piperazine;

1-{2-(3-Aminopropionamido)-3-(2,4-dichlorophenyl)propionyl}-4-{1-[(2-fluorobenzylamino)ethyl]cyclohexyl}piperazine;

1-{2-(3-Aminopropionamido)-3-(2,4-dichlorophenyl)propionyl}-4-{1-[(benzoylamino)ethyllcyclohexyl}piperazine;

1-{2-(3-Aminopropionamido)-3-(2,4-dichlorophenyl)propionyl}-4-{1-[(phenylsulfonamido)ethyl]cyclohexyl}piperazine;

1-{[2-(3-Aminopropionamido)-3-(2,4-dichlorophenyl)propionyl}-4-{1-[(phenylureido)ethyl]cyclohexyl}piperazine;

1-{2-(1,2,3,4-Tetrahydro-isoquinoline-3-carboxamido)-3-(4-chlorophenyl)propionyl}-4-{1-[phenylacetamidomethyl]cyclohexyl}piperazine;

1-{2-(1-Amino-indan-1-carboxamido)-3-(4-chlorophenyl)propionyl}-4-{1-[phenylacetamidomethyl]cyclohexyl}piperazine;

1-{2-(3-Amino-3-phenylpropionamido)-3-(4-chlorophenyl)propionyl}-4-{1-[phenylacetamidomethyl]cyclohexyl}piperazine;

1-{2-(1,2,3,4-Tetrahydro-isoquinoline-1-carboxamido)-3-(4-chlorophenyl)propionyl}-4-{1-[phenylacetamidomethyl]cyclohexyl}piperazine;

1-{2-(2-Amino-2-phenylacetamido)-3-(4-chlorophenyl)propionyl}-4-{1-[phenylacetamidomethyl]cyclohexyl}piperazine;

1-{2-(Quinoline-3-carboxamido)-3-(4-chlorophenyl)propionyl}-4-{1-[phenylacetamidomethyllcyclohexyl}piperazine;

1-{2-[2-Amino-3-(2-pyridyl)propionamido]-3-(4-chlorophenyl)propionyl}-4-{1-[phenylacetamidomethyl]cyclohexyl}piperazine;

1-{[2-[2-Amino-3-(3-pyridyl)propionamido]-3-(4-chlorophenyl)propionyl}-4-{1-[phenylacetamidomethyl]cyclohexyl}piperazine; and

1-{2-[2-Amino-3-(4-pyridyl)propionamido]-3-(4-chlorophenyl)propionyl}-4-{1-[phenylacetamidomethyl]cyclohexyl}piperazine.

The compounds of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, ftimaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Compounds of structure (I) may also possess axial chirality which may result in atropisomers. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The compounds of this invention may be evaluated for their ability to bind to a MC receptor by techniques known in this field. For example, a compound may be evaluated for MC receptor binding by monitoring the displacement of an iodonated peptide ligand, typically [¹²⁵I]-NDP-α-MSH, from cells expressing individual melanocortin receptor subtypes. To this end, cells expressing the desired melanocortin receptor are seeded in 96-well microtiter Primaria-coated plates at a density of 50,000 cells per well and allowed to adhere overnight with incubation at 37° C. in 5% CO₂. Stock solutions oftest compounds are diluted serially in binding buffer (D-MEM, 1 mg/ml BSA) containing [¹²⁵I]-NDP-α-MSH (10⁵ cpm/ml). Cold NDP-A-MSH is included as a control. Cells are incubated with 50 μl of each test compound concentration for 1 hour at room temperature. Cells are gently washed twice with 250 μl of cold binding buffer and then lysed by addition of 50 μl of 0.5 M NaOH for 20 minutes at room temperature. Protein concentration is determined by Bradford assay and lysates are counted by liquid scintillation spectrometry. Each concentration of test compound is assessed in triplicate. IC₅₀ values are determined by data analysis using appropriate software, such as GraphPad Prizm, and data are plotted as counts of radiolabeled NDP-MSH bound (normalized to protein concentration) versus the log concentration of test compound.

In addition, functional assays of receptor activation have been defined for the MC receptors based on their coupling to Gₛ proteins. In response to POMC peptides, the MC receptors couple to G_{S} and activate adenylyl cyclase resulting in an increase in cAMP production. Melanocortin receptor activity can be measured in HEK293 cells expressing individual melanocortin receptors by direct measurement of cAMP levels or by a reporter gene whose activation is dependent on intracellular cAMP levels. For example, HEK293 cells expressing the desired MC receptor are seeded into 96-well microtiter Primaria-coated plates at a density of 50,000 cells per well and allowed to adhere overnight with incubation at 37° C. in 5% CO₂ Test compounds are diluted in assay buffer composed of D-MEM medium and 0.1 mM isobutylmethylxanthine and assessed for agonist and/or antagonist activity over a range of concentrations along with a control agonist α-MSH. At the time of assay, medium is removed from each well and replaced with test compounds or α-MSH for 30 minutes at 37° C. Cells are harvested by addition of an equal volume of 100% cold ethanol and scraped from the well surface. Cell lysates are centrifuged at 8000×g and the supernatant is recovered and dried under vacuum. The supernatants are evaluated for cAMP using an enzyme-linked immunoassay such as Biotrak, Amersham. EC₅₀ values are determined by data analysis using appropriate software such as GraphPad Prizm, and data are plotted as cAMP produced versus log concentration of compound.

As mentioned above, the compounds of this invention function as ligands to one or more MC receptors, and are thereby useful in the treatment of a variety of conditions or diseases associated therewith. In this manner, the ligands function by altering or regulating the activity of an MC receptor, thereby providing a treatment for a condition or disease associated with that receptor. In this regard, the compounds of this invention have utility over a broad range of therapeutic applications, and may be used to treat disorders or illnesses, including (but not limited to) eating disorders, cachexia, obesity, diabetes, metabolic disorders, inflammation, pain, skin disorders, skin and hair coloration, male and female sexual dysfunction, erectile dysfunction, dry eye, acne and/or Cushing's disease.

The compounds of the present invention may also be used in combination therapy with agents that modify sexual arousal, penile erections, or libido such as sildenafil, yohimbine, apomorphine or other agents. Combination therapy with agents that modify food intake, appetite or metabolism are also included within the scope of this invention. Such agents include, but are not limited to, other MC receptor ligands, ligands of the leptin, NPY, melanin concentrating hormone, serotonin or B₃ adrenergic receptors.

In another embodiment, pharmaceutical compositions containing one or more compounds of this invention are disclosed. For the purposes of administration, the compounds of the present invention may be formulated as pharmaceutical compositions. Pharmaceutical compositions of the present invention comprise a compound of structure (I) and a pharmaceutically acceptable carrier and/or diluent. The compound is present in the composition in an amount which is effective to treat a particular disorder of interest, and preferably with acceptable toxicity to the patient. Typically, the pharmaceutical composition may include a compound of this invention in an amount ranging from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface active agents, binders, and lubricants. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating a condition related to an MC receptor. Such methods include administration of a compound of the present invention to a warm-blooded animal in an amount sufficient to treat the condition. In this context, “treat” includes prophylactic administration. Such methods include systemic administration of compound of this invention, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration, not limitation.

### EXAMPLES

Aqueous Work Up

The reaction mixture was concentrated under a stream of nitrogen, taken up in dichloromethane, washed with aqueous sodium bicarbonate, and again concentrated. Final compounds were dissolved in methanol and filtered prior to preparative HPLC purification.

HPLC Columns and Gradients

Analytical HPLC columns were BHK laboratories ODS/0/13 30×75 mm, 5 μm, 120 A; the standard gradient was 1 mL/min 10-90% CH₃CN in water over 2 minutes, then 90% CH₃CN for 1 minute. Constant percentage of 0.1% TFA was added.

Prep HPLC column

YMC AQ, 5 μm, 120 A20, 20×50 mm cartridges

### Example 1

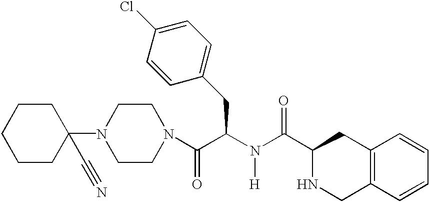

Step 1A: Synthesis of Nitrile

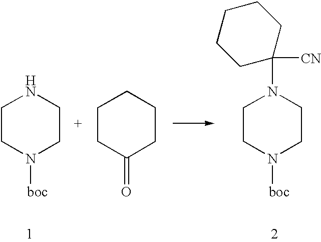

Cyclohexanone (27 mmol) was dissolved in water (80 mL) and treated with sodium metabisulfite (2.57 g, 13.5 mmol). The mixture was stirred for 90 min and the protected piperazine 1 (27 mmol) was added. After an additional 2 h, sodium cyanide (1.38 g, 28.2 mmol) was added and stirring was continued for 20 h. The mixture was extracted three times with dichloromethane (30 mL), the extracts were combined, dried (MgSO₄), and concentrated to afford 2.

Step 1B: Deprotection

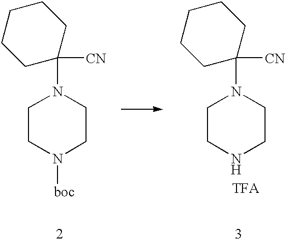

Compound 2 was dissolved in dichloromethane, treated with an equal volume of anhydrous trifluoroacetic acid and stirred 0.5 hours at room temperature. The solvent was removed in vacuo. The compound was suspended in dichloromethane, the solvent removed and the residue pumped under high vacuum to give compound 3.

Step 1C: Peptide Coupling

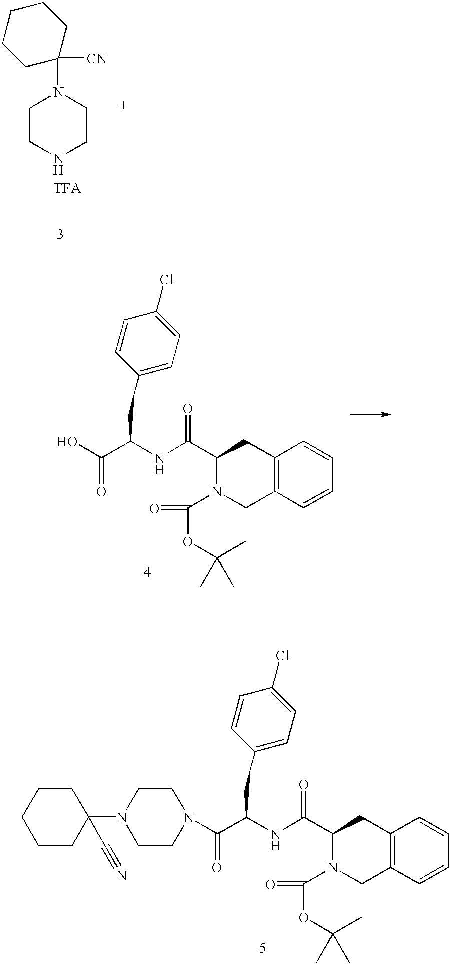

Dipeptide 4 (100 mg) was dissolved in CH₂Cl₂ (4 mL) and was treated with 80 uL of DIEA. HBTU (206 mg) was added and the reaction stirred 30 minutes. The piperazine-TFA salt 3 was added in 1 mL dry CH₂Cl₂ and the reaction stirred 60 hours. The reaction mixture was diluted with CH₂Cl₂ and was washed with 10% sodium bicarbonate solution, water and saturated sodium chloride solution. The organic layer was dried over anhydrous sodium sulfate and concentrated in vacuo to give oil 5.

Step 1D: Deprotection and Purification

Dipeptide 5 was dissolved in 500 uL of CH₂Cl₂ and was treated with 500 uL anhydrous TFA. The reaction was stirred for 30 minutes at room temperature and was concentrated. A portion of this material was purified using preparative thin layer chromatography eluting with a mixture of methanol and dichloromethane. The compound of Example 1 was obtained after extraction from the silica as a colorless oil. RT=2.763 min (gradient A), LC-MS (M−CN)+=507.

### Example 2

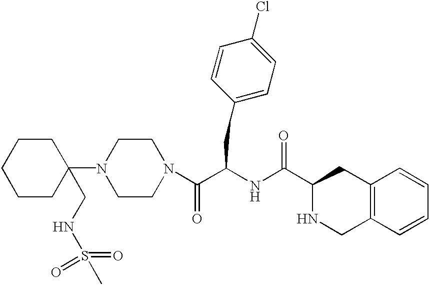

Step 2A: Sulfonamide

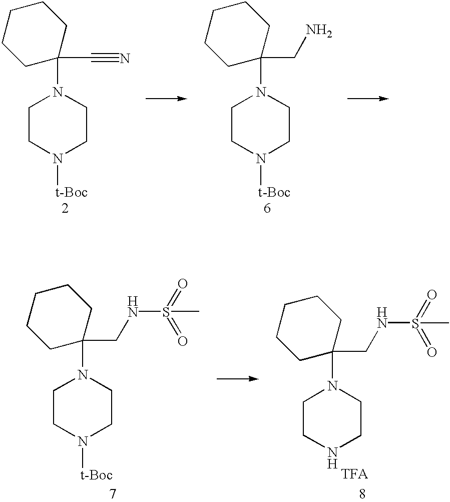

The nitrile 2 (0.853 mmol) was dissolved in THF (5 mL) and LiAlH₄ (161 mg, 4.26 mmol) was added at 0° C. The reaction was brought to room temperature and stirred for 30 minutes. The mixture was cautiously treated with water (0.16 mL), 15% aqueous sodium hydroxide (0.16 mL), and water (0.48 mL) with vigorous stirring. The mixture was filtered and the filtrate concentrated to afford the crude amine. This material (0.11 mmol) was dissolved in dichloromethane (1 mL), treated with triethylamine (0.15 mmol) and methanesulfonyl chloride (0.15 mmol), and the resulting mixture was stirred for 18 h. Workup according to procedure A produced the desired BOC-protected sulfonamide 7.

Sulfonamide 7 (0.338 mmole) was dissolved in 1 mL 1:1 dichloromethane:trifluroacetic acid, after 1 hour the solvent was removed in vacuo and the residue was suspended in 1 mL of dichloromethane and evaporated to dryness under high vacuum to provide TFA salt 8.

Step 2B: Deprotection and Purification

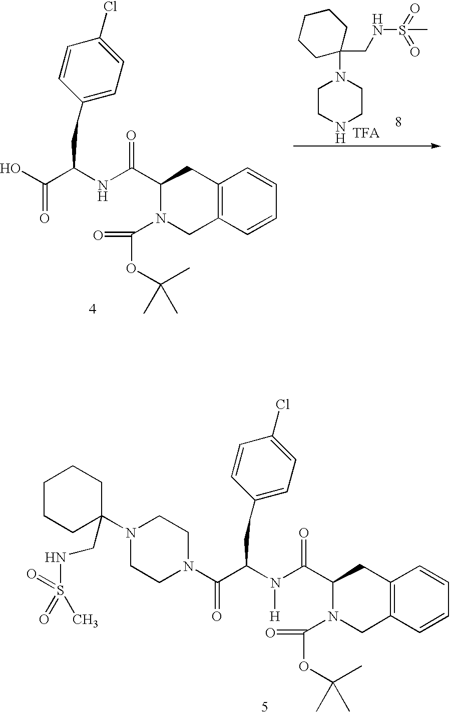

Protected dipeptide fragment 4 (0.05 mmole) was dissolved in 300 uL of dichloromethane, and 20 uL of N-diisopropyl-N-ethyl amine was added followed by HBTU. After 30 minutes the TFA piperidine salt 8 (0.05 mmole) was added in 500 uL dichloromethane and was stirred for approximately 15 hours. Aqueous work-up provided dipeptide 9.

Dipeptide 9 was dissolved in 500 uL of C14₂Cl₂ and was treated with 500 uL anhydrous TFA. The reaction was stirred for 30 minutes at room temperature and was concentrated in vacuo. A portion of this material was dissolved in CH₃CN and was purified using preparative C₁₈ HPLC-MS chromatography eluting with a gradient of acetonitrile in water containing 0.1% TFA. The compound of Example 2 was obtained as a colorless oil as the TFA salt after evaporation of solvent. RT=2.419 min (gradient A), LC-MS (M+H)=616.

### Example 3

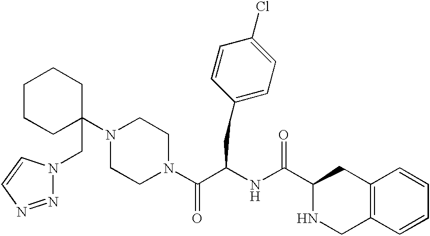

Step 3A: Synthesis of N-methanesulfonic 2,2-dichloroethylidene Hydrazide

Mesylhydrazine (100 mg) was dissolved in 1.5 mL of propionic acid and was treated with dichloroacetaldehyde at 0° C. After stirring for 1 hour at 0° C., the white solid was collected by filtration and washed with toluene to provide the title compound.

Step 3B: Synthesis of 1.2,3 Triazole

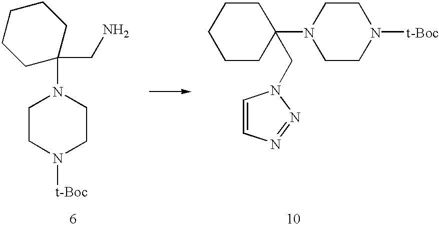

Amine 6 (0.58 mmole) was dissolved in 500 uL of methanol and 140 uL of triethylamine was added and the mixture was cooled to 0° C. N-Methanesulfonic 2,2-dichlorethylidene hydrazide (100 mg) in 500 uL MeOH was added dropwise. The reaction was then heated to 50° C., and was stirred at this temperature for 15 hours. The reaction mixture was then concentrated in vacuo, dissolved in dichloromethane and washed with saturated sodium bicarbonate solution and saturated NaCl solution. The mixture was dried over anhydrous sodium sulfate and concentrated in vacuo to provide triazole 10 as an oil.

Step 3C: Deprotection and Coupling

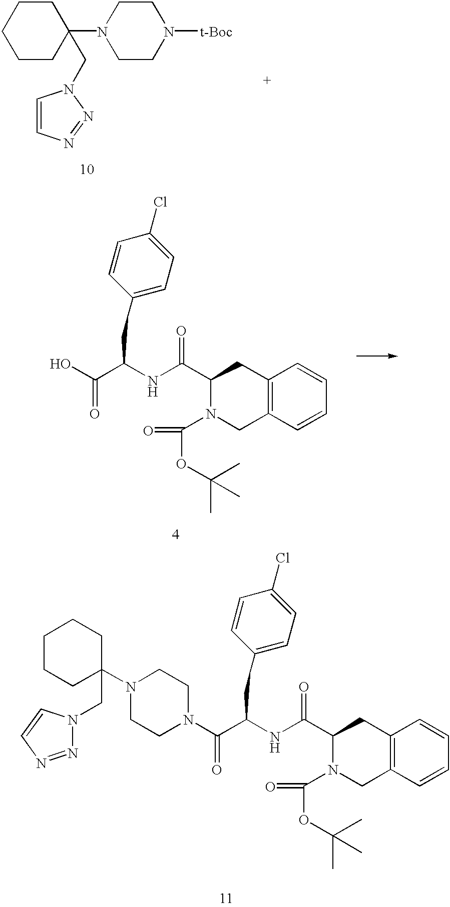

Triazole 10 (˜0.58 mmole) was dissolved in 2 mL 1:1 dichloromethane:trifluoroacetic acid, after 30 minutes the solvent was removed in vacuo and the residue was suspended in 1 mL of dichloromethane and evaporated to dryness under high vacuum to provide TFA salt 10a.

Protected dipeptide fragment 4 (240 mg) was dissolved in 1.5 mL of dichloromethane, and 0.34 mL of N-diisopropyl-N-ethyl amine was added followed by HBTU (385 mg). After 30 minutes, a solution of the TFA piperidine salt 10a (240 mg) in 1 mL dichloromethane was added and stirred approximately 15 hours. Aqueous work-up provided dipeptide 11.

Step 3D: Deprotection and Purification

Dipeptide 11 was dissolved in 500 FL of CH₂Cl₂ and treated with 500 uL anhydrous TFA. The reaction stirred 30 minutes at room temperature and was concentrated in vacuo. A portion of this material was dissolved in CH₃CN and purified using preparative C₁₈ HPLC-MS chromatography eluting with a gradient of acetonitrile in water containing 0.1% TFA. The compound of Example 3 was obtained as the TFA salt as a colorless oil after evaporation of solvent. RT=2.428 min (gradient A), LC-MS (M+H)=590.

### Example 4

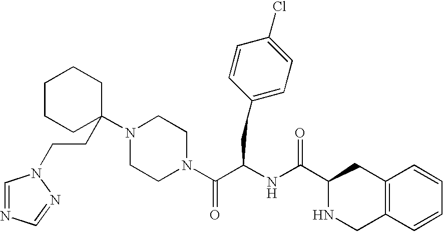

Step 4A:

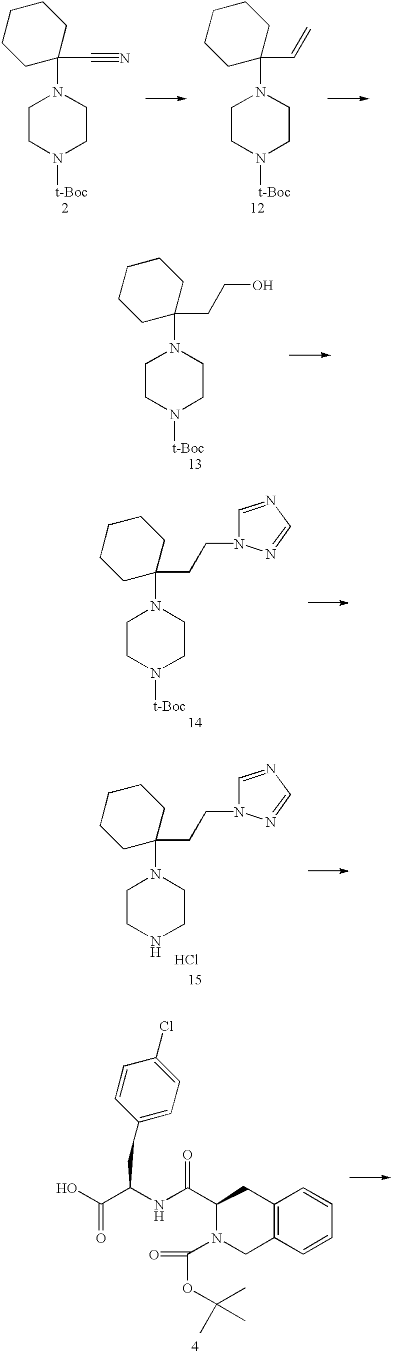

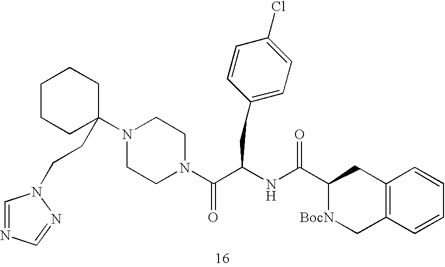

Nitrile 2 (500 mg) was dissolved in 3 mL of dry THF and was cooled to 0° C. under nitrogen atmosphere. A 1 M solution of vinyl magnesium bromide (5 mL) was added dropwise via syringe over 5 minutes. The cooling bath was removed and the reaction stirred for 3 hours. The mixture was cooled to 0° C. and was quenched by the slow, careful addition of 8 mL of saturated NH₄Cl solution. The mixture was extracted three times with ethyl acetate; the organic layers were combined and washed with saturated sodium chloride solution and dried over anhydrous sodium sulfate. Removal of the solvent in vacuo provided crude alkene 12 (500 mg).

Step 4B:

Alkene 12 (260 mg) was dissolved in 6 mL of dry THF and treated slowly under nitrogen with a 1M solution BH₃-THF in THF (4.5 mL). The reaction was heated at reflux for 15 hours, allowed to cool and concentrated in vacuo. MeOH (6 mL) was added cautiously, and concentrated. Again MeOH (6 mL) was added and concentrated. The mixture was then dissolved in 4 mL THF and ˜300 μL of 4 N NaOH was added followed by a H₂O₂ (30% solution, 500 μL). The reaction stirred for two hours at room temperature and was diluted with a few mL of water and extracted with EtOAc. The combined organic layers were washed with water and saturated sodium chloride solution and concentrated to crude alcohol 13 (170 mg).

Step 4C:

A portion of the alcohol 13 (80 mg) was dissolved in THF (2 mL) followed by triphenylphospine (90 mg) and diisopropylazo-dicarboxylate (DIAD 70 FtL) and was stirred for 5 minutes. 1,2,4-Triazole (20 mg) was added and the reaction was stirred for 15 hours. An additional 90 mg of triphenyl phosphine and DIAD (70 μL) were added, stirred 5 minutes and then 1.2,4 triazole (60 mg) was added. The mixture stirred an additional three hours. Extractive work-up according to method A provided crude product 14. This material was dissolved in dichloromethane (2 mL) and was treated with TFA (2 mL). After 30 minutes the solvent was removed in vacuo. In order to remove triphenyl phosphine the product was dissolved in dichloromethane and was then stirred with 10% K₂CO₃ solution. The aqueous solution was extracted with dichloromethane solution. All organic layers were combined, dried carefully over anhydrous sodium sulfate and concentrated to a very small volume. Anhydrous diethyl ether was added followed by 345 μL of 2M HCl in ether. The HCl salt 15 was collected and used without further purification.

Step 4D:

Dipeptide 4 (70 mg) was dissolved in dichloromethane (3 mL) and was treated with DIEA (55 μL) and HBTU (61 mg) and the mixture was stirred for 15 minutes. HCl salt 15 was dissolved in minimum amount of dichloromethane and was added. The reaction was stirred overnight. Normal extractive work up method A provided crude compound 16. This material was dissolved in 1 mL CH₂Cl₂ and was treated with 1 mL anhydrous TFA, after 30 minutes the solvent was removed in vacuo.

A portion of this material was dissolved in CH₃CN and was purified using preparative C₁₈ HPLC-MS chromatography eluting with a gradient of acetonitrile in water containing 0.1% TFA. The compound of Example 4 was obtained as a colorless oil as the TFA salt after evaporation of the solvent. RT=2.406 min (gradient A), LC-MS (M+H)=604.

### Example 5

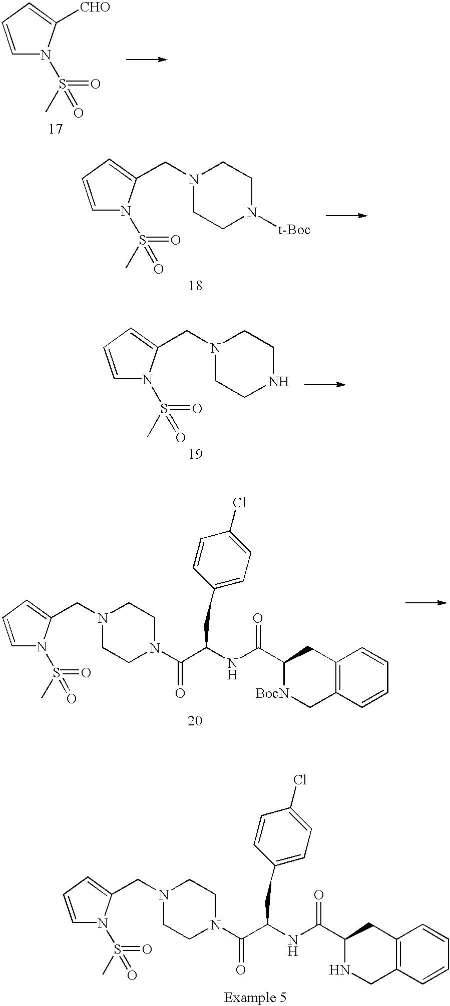

Step 5A:

Pyrrole-2-carboxaldehyde (1.01 g) was dissolved in dry THF (15 mL) and was treated with sodium hydride (300 mg). The reaction was stirred under nitrogen for 10 minutes then mesyl chloride (0.53 mL) was added. The reaction was stirred for 2 hours at room temperature then NaH (100 mg) and mesyl chloride (0.20 mL) were added and the reaction was stirred an additional 2 hours. The mixture was quenched with water and extracted with ethyl acetate. The extracts were combined and dried over anhydrous magnesium sulfate and were concentrated to provide crude 17 (261 mg) as a dark oil.

Step 5B:

Aldehyde 17 (99 mg) and Boc-piperazine (117 mg) were dissolved in dry acetonitrile and stirred for five minutes. Sodium triacetoxyborohycride was added and the mixture stirred for 18 hours at room temperature. The mixture was concentrated under a stream of nitrogen and was dissolved in dichloromethane (4 mL) and 4 mL of TFA. After stirring 1 hour the mixture was concentrated under a stream of nitrogen, dissolved in 4 mL of dichloromethane and was washed with saturated NaHCO₃ solution. The organic layer was dried over anhydrous magnesium sulfate and concentrated to afford crude piperazine 19_(144 mg) as an oil.

Step 5C:

Dipeptide 4 (182 mg) and piperidinie 19 were dissolved in a mixture of 1.5 mL dichloroniethane and 0.4 mL NMP. HOBt (48 mg) and EDC (67 mg) were added and the reaction stirred at room temperature 15 hours. Extractive work up A provided the crude compound 20.

Step 5D:

Compound 20 was dissolved in 1 mL of dichloromethane and treated with 1 mL of anhydrous TFA, after 30 minutes the solvent was removed in vacuo. A portion of this material was dissolved in CH₃CN and purified using preparative C₁₈ HPLC-MS chromatography eluting with a gradient of acetonitrile in water containing 0.1% TFA. The compound of Example 5 was obtained as a colorless oil as the TFA salt after evaporation of solvent. RT=2.332 min (gradient A), LC-MS (M+H)=584.

### Example 6

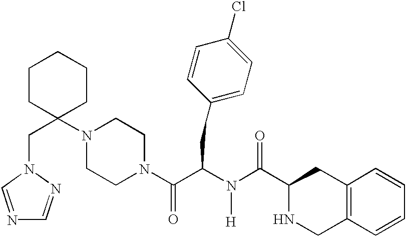

Step 6A: Synthesis of Nitrile

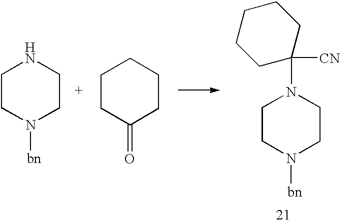

Cyclohexanone (5.90 mL, 56.9 mmol) and sodium metabisulfite (9.80 g, 51.6 mmol) were dissolved in water (200 mL) and stirred for 1 hour. Benzyl 1-piperazinecarboxylate (11.0 mL, 57.0 mmol) was added and stirring was continued for 2 h. Sodium cyanide (2.79 g, 56.9 mmol) was added and the mixture was stirred for 16 h and then was extracted with dichloromethane. The combined extracts were dried (MgSO₄) and concentrated under vacuum to afford 16.4 g (100%) of 21 as a white solid: LCMS (MH⁺−HCN, 257).

Step 6B: Reduction to Amine

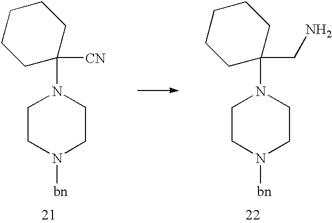

Nitrile 21 (2.12 g, 7.48 mmol) was dissolved in THF (50 mL) and was cooled to 0° C. LAH (1.42 g, 37.4 mmol) was added in portions over 15 min. Upon completion of the addition, the ice-bath was removed and stirring was continued for 18 h. The mixture was cooled in an ice-bath and treated cautiously with water (1.4 mL), 15% aqueous sodium hydroxide (1.4 mL) and water (4.3 mL) and stirring was continued for 30 minutes at rt. The mixture was dried (MgSO₄), filtered, and the solid washed liberally with ethyl acetate. The combined filtrates were concentrated under vacuum to afford 1.97 g (92%) of 22 as a colorless oil. LCMS (MH⁺, 288).

Step 6C: Synthesis of Triazole

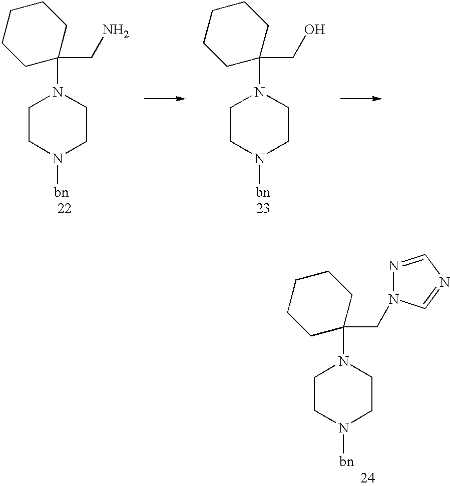

Amine 22 (630 mg, 2.19 mmol) was suspended in water (5 mL) and the pH was adjusted to 10 by the addition of 15% aqueous sodium hydroxide. Sodium nitroferricyanide dihydrate (979 mg, 3.29 mmol) was added and the mixture was heated at 60° C. for 8 h, with the pH being maintained above 9 by the occasional addition of aqueous sodium hydroxide. The mixture was cooled to rt, filtered (Celite), and the resulting solution was extracted with dichloromethane. The combined extracts were dried (MgSO₄) and concentrated under vacuum to afford the crude alcohol 23.

The above material was dissolved in dichloromethane (5 mL), cooled in an ice-bath and treated with triethylamine (0.17 mL, 1.2 mmol) and methanesulfonyl chloride (0.062 mL, 0.80 mmol). The ice-bath was removed and the mixture was stirred for 1 h, washed with water, dried (MgSO₄) and filtered. Sodium triazole (182 mg, 2.00 mmol) was added and the mixture was heated at 50° C. in a sealed vial for 20 h. The mixture was cooled, filtered, and concentrated under vacuum. The residue was purified by preparative HPLC to afford 60 mg of the TFA salt of 24 as a colorless oil.

Step 6D: Removal of Benzyl Protecting Group

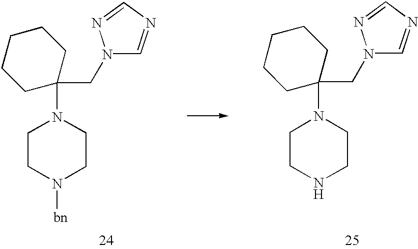

Triazole 24 (32 mg, 0.071 mmol), ammonium formate (15 mg, 0.24 mmol) and 10% palladium on charcoal (15 mg) were combined in ethanol (0.5 mL) and heated at 80° C. in a sealed vial for 90 minutes. The mixture was cooled, concentrated in vacuo, taken up in methanol (1 mL) and filtered (Celite). The methanol solution was then concentrated under vacuum to afford 11 mg (33%) of the TFA salt of 25, which was used without further purification.

Step 6E: Peptide Coupling and Removal of BOC Protecting Group

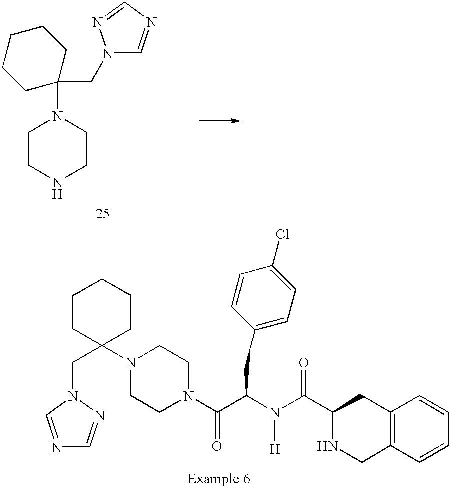

Triazole 25 (11 mg, 0.024 mmol) was dissolved in dichloromethane (0.5 mL) and was treated with triethylamine (0.028 mL, 0.20 mmol), boc-D-tic-D-Cl-phe-OH (22 mg, 0.048 mmol) and HOBt (7 mg, 0.052 mmol). The mixture was stirred for 10 min and then treated with EDC (10 mg, 0.052 mmol). It was stirred for 20 h, washed with aqueous sodium bicarbonate, treated with TFA (0.5 mL) and stirred for 45 min. The mixture was concentrated under a stream of nitrogen and the residue was purified by preparative HPLC to afford The compound of Example 6 as a white solid. RT=2.623 min (gradient A), LC-MS (M+H)=590.

### Example 7

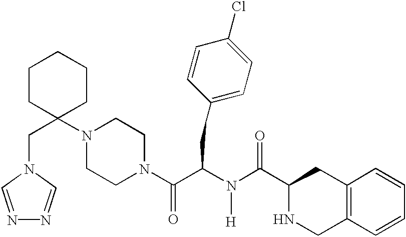

Step 7A: Triazole Formation

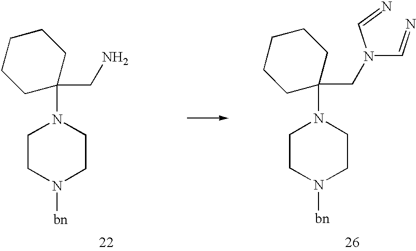

Amine 22 (223 mg, 0.78 mmol) and N,N-dimethylformamidine azine dihydrochloride (172 mg, 0.80 mmol) were combined in DMF (2 mL) and heated at 150° C. for 18 h. The mixture was cooled, diluted with ethyl acetate (10 mL), and washed four times with aqueous sodium chloride. The organic extracts were dried (MgSO₄), concentrated and the residue was purified by prep HPLC to afford 83 mg (23%) of the TFA salt of 26 as a colorless oil: LCMS (MH⁺, 340).

Step 7B: Benzyl Deprotection, Peptide Coupling, and BOC Deprotection

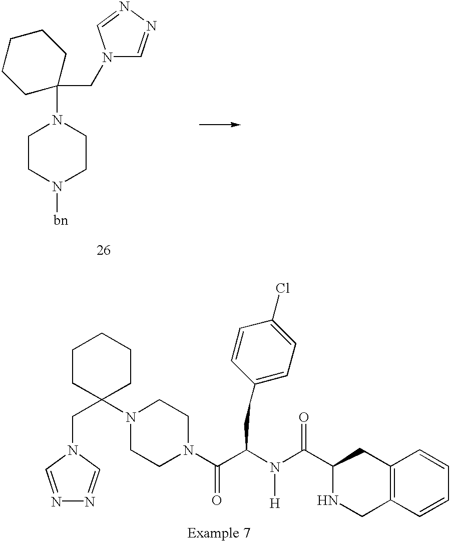

Triazole 26 was elaborated to the compound of Example 7 in an analogous manner as in the conversion of 24 to the compound of Example 6. The compound of Example 7: RT=2.479 min (gradient A), LC-MS (M+H)=590.

### Example 8

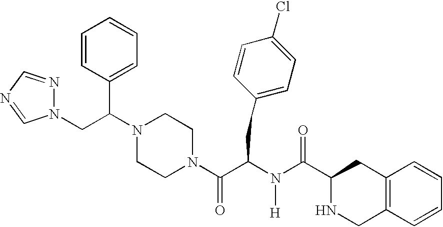

Step 8A: Synthesis of 27

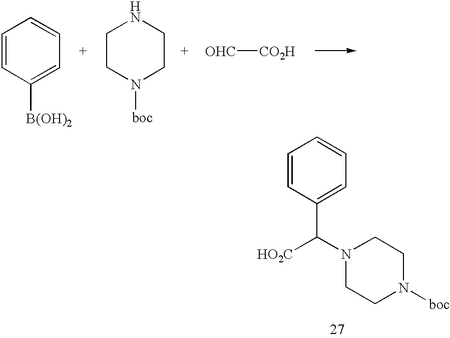

t-Butyl 1-piperazinecarboxylate (100 mg, 0.54 mmol), glyoxylic acid monohydrate (50 mg, 0.54 mmol), and benzeneboronic acid (66 mg, 0.54 mmol) were heated at 50° C. in ethanol (2 mL) for 20 h. The mixture was cooled and concentrated in vacuo to afford the crude acid 27 as a white solid. LCMS (MH⁺, 321).

Step 8B: Synthesis of Triazole

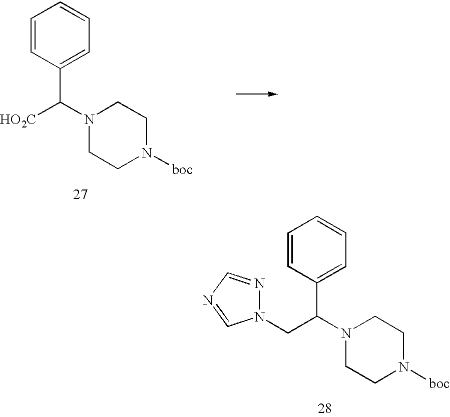

Carboxylic acid 27 (173 mg, 0.54 mmol) and triethylamine (0.090 mL, 0.64 mmol) were dissolved in THF (5 mL) and cooled to 0° C. Ethyl chloroformate (0.062 mL, 0.64 mmol) was added, the ice-bath was removed and stirring was continued for 2 h. The mixture was filtered and the resulting solution was added to an ice-cooled, stirred suspension of sodium borohydride (82 mg, 2.2 mmol) in water (1 mL). The mixture was stirred for 1 h at 0° C. and then diluted with water (5 ml,). It was then extracted with ethyl acetate and the combined extracts were dried (MgSO₄) and concentrated to afford the crude alcohol, which was used without further purification. This material was converted to triazole 28 using the same procedure for the conversion of 2 into 2.

Step 8C: Synthesis of Dipeptide

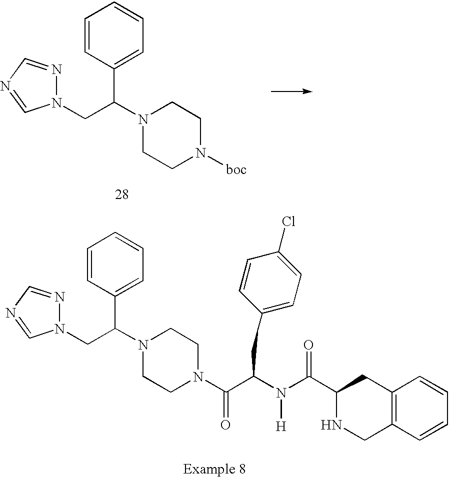

Triazole 28 (30 mg, 0.083 mmol) was dissolved in dichloromethane (0.5 mL), treated with TFA (0.5 mL) and stirred for 45 minutes. The mixture was concentrated under vacuum to afford the TFA salt of the deprotected piperazine that was elaborated to the compound of Example 8 in an analogous manner as in the conversion of 2 to the compound of Example 6. The compound of Example 8: RT=2.283 min (gradient A), LC-MS (M+H)=598.

By the general procedures set forth above, the following compounds were also made.

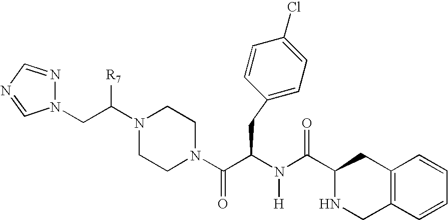

| Example | R₇ | MW | MS ion | Retention |
|---|---|---|---|---|
| 8-1 | Ph | 598.1 | 598 | 2.283 |
| 8-2 | 4-OMe-Ph | 628.2 | 628 | 2.299 |
| 8-3 | 1-Naphthyl | 648.2 | 548 | 2.708 |
| 8-4 | 4-SMe-Ph | 644.2 | 644 | 2.676 |
| 8-5 | 2-Naphthyl | 648.2 | 648 | 2.709 |
| 8-6 | 4-t-Butyl-Ph | 654.3 | 654 | 2.547 |
| 8-7 | 3-Ph-Ph | 674.2 | 674 | 2.541 |
| 8-8 | 5-Isopropyl-2-OMe-Ph | 670.3 | 670 | 2.503 |
| 8-9 | 2,5-Dimethyl-Ph | 626.2 | 626 | 2.435 |
| 8-10 | Ph-CH₂CH₂— | 626.2 | 626 | 2.4 |
| 8-11 | 2-Furan | 592.1 | 592 | 2.209 |

### EXAMPLE 9

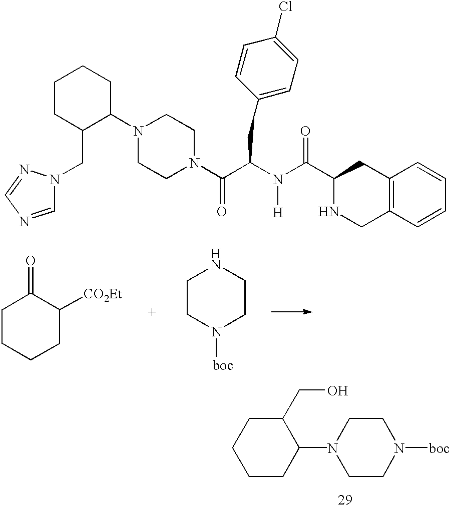

Step 9A:

t-Butyl 1-piperazinecarboxylate (5.08 g, 27.3 mmol), ethyl 2-cyclohexanonecarboxylate (4.35 mL, 27.2 mmol) and acetic acid (10 drops) were dissolved in DMF (25 mL) and stirred for 20 min. Sodium cyanoborohydride (2.41 g, 38.4 mmol) was added and the mixture was heated at 55° C. for 16 h. The reaction mixture was cooled, poured into ethyl acetate (75 mL) and washed with water (75 mL) and aqueous sodium chloride (3×75 mL). The organic layer was dried (MgSO₄) and concentrated in vacuo to afford 6.26 g of the crude ester. A portion of this material (2.02 g, ca 5.93 mmol) was dissolved in THF (5 mL) and added to an ice-cooled, stirred suspension of LAH (1.13 g, 29.8 mmol) in THF (10 mL). Once the addition was complete, the ice-bath was removed and stirring was continued for 1 h. The mixture was treated cautiously with water (1.1 mL), 15% aqueous sodium hydroxide (1.1 mL), and water (3.4 mL) with vigorous stirring. The resulting suspension was dried (MgSO₄), filtered, and concentrated under vacuum to afford the 1.79 g of crude 29 as a yellow oil. LCMS (MH⁺, 299).

Step 9B: Synthesis of Triazole

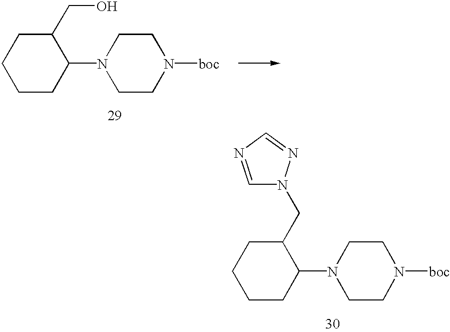

Alcohol 29 was converted to triazole 30 in an analogous manner to the conversion of 23 to 24.

Step 9C: Synthesis of Dipeptide

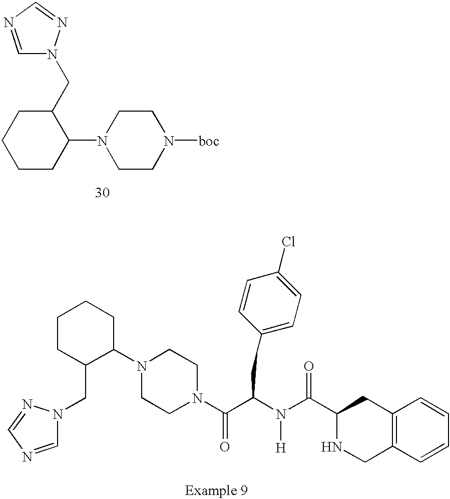

Triazole 30 was converted to the compound of Example 9 using the same procedure as for the conversion of 28 to the compound of Example 8. The compound of Example 9: RT=2.389 min (gradient A), LC-MS (M+H)=590.

By the general procedures set forth above, the following compounds were also made.

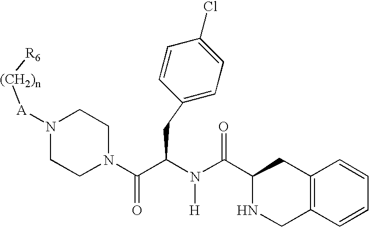

| Example | —A—(CH₂)ₙ—R₆ | MW | MS ion | Retention |
|---|---|---|---|---|
| 9-1 | 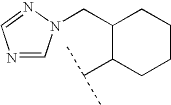 | 590.2 | 590 | 2.389 |
| 9-2 | 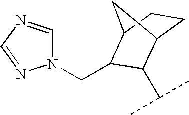 | 602.2 | 602 | 2.134 |
| 9-3 | 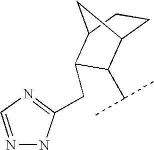 | 602.2 | 602 | 2.163 |
| 9-4 | 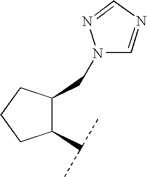 | 576.1 | 576 | 2.349 |
| 9-5 | 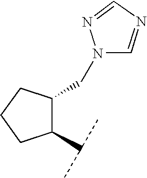 | 576.1 | 576 | 2.338 |

Using (1S,4S)-2,5-diazabicyclo[2.2.1]heptane in place of t-butyl piperazine carboxylate as a starting material gave the following compound.

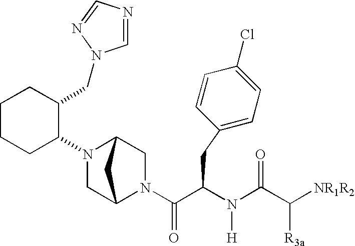

| Example | —CHR₃ₐNR₁R₂ | MW | MS ion | Retention |
|---|---|---|---|---|
| 9-6 | 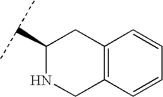 | 602.2 | 602 | 2.396 |

### Examples 10-13

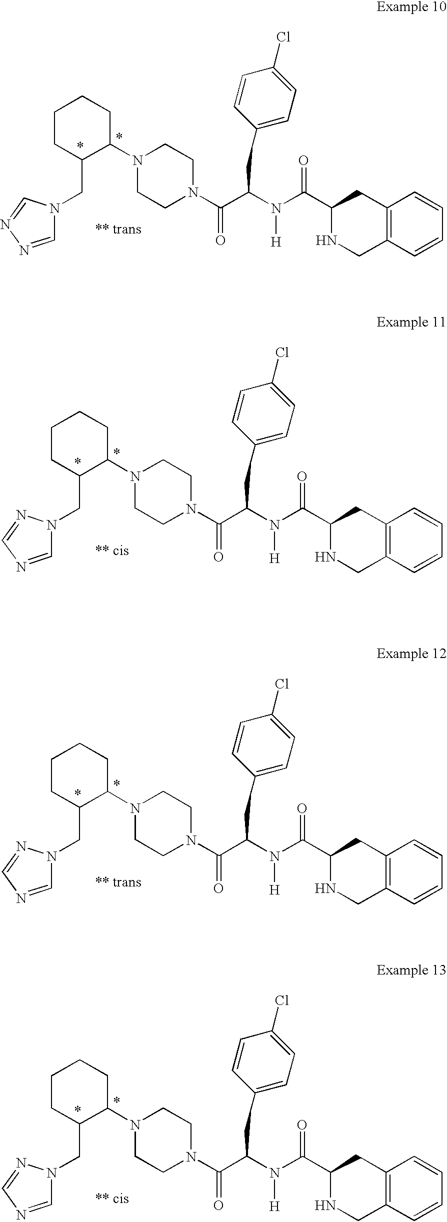

Step 10A: Synthesis of Keto-Triazoles

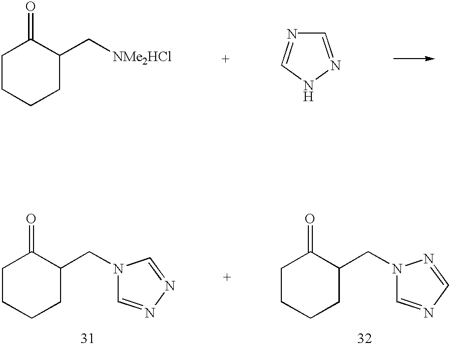

Triazole (9.01 g, 130 mmol) and 2-(dimethylaminomethyl)-1-cyclohexanone (5.00 g, 26.0 mmol) were refluxed in 1:1 ethanol-water (80 mL) for 4 h. The mixture was concentrated, taken up in dichloromethane (30 mL), washed with aqueous sodium bicarbonate, dried (MgSO₄) and again concentrated. The residue was purified on a silica gel column (elution with 1-5% methanol in dichloromethane) to afford 2.04 g (44%) of 32 as a colorless oil and 0.759 g (16%) of 31 as awhite powder. Triazole 31: LCMS (MH⁺, 180). Triazole 32: LCMS (MH⁺, 180).

Step 10B: Reductive Amination

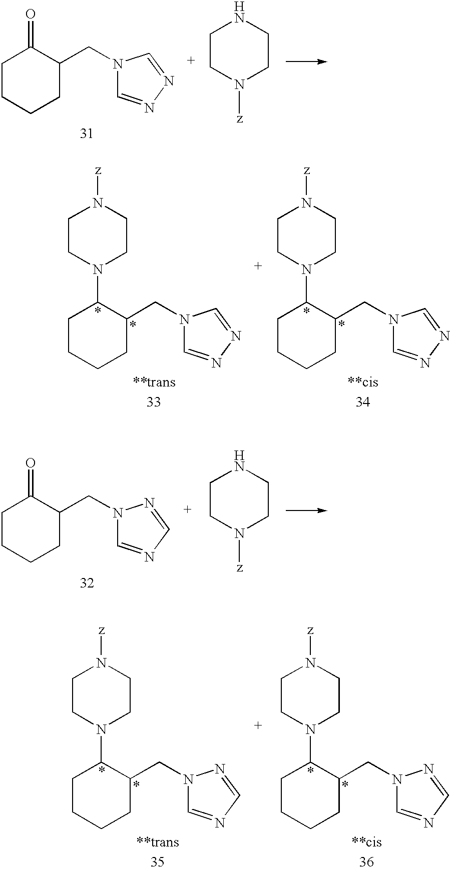

Ketone 31 (100 mg, 0.56 mmol) and benzyl 1-piperazinecarboxylate (0.32 mL, 1.66 mmol) were dissolved in dichloromethane (6 mL) and cooled to 0° C. A 1.0 M solution oftitanium(IV) chloride in dichloromethane (0.56 mL, 0.56 mmol) was added and the mixture was stirred at 0° C. for 30 min. and 3 h at rt. A solution of sodium cyanoborohydride (141 mg, 2.24 mmol) in isopropanol (6 mL) was added and stirring was continued for 20 h. Water (1 mL) was added and the mixture was stirred for 5 min. and filtered. The filtrate was concentrated and the residue was taken up in dichloromethane, washed with aqueous sodium chloride, dried (MgSO₄) and again concentrated. The residue was purified by preparative HPLC to afford 28 mg (10%) of the TFA salt of 33 and 22 mg (8%) of the TFA salt of 34, both as colorless oils.

Triazoles 35 and 36 were prepared in a similar fashion from 32.

Step 10C

### Synthesis of Examples 10-13

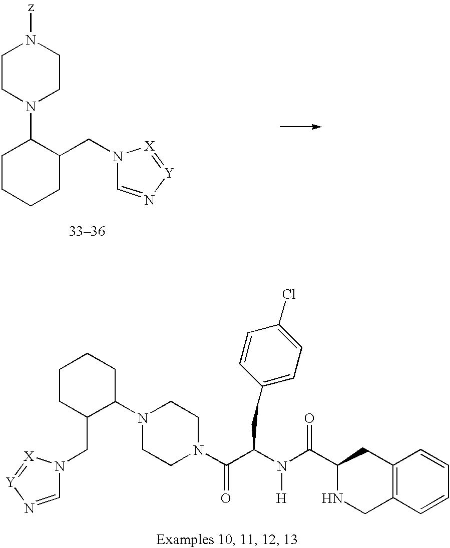

The compounds of Examples 10-13 were synthesized from triazoles 33 through 36, respectively, in an analogous manner as in the conversion of 24 to the compound of Example 6. The compound of Example 10: RT=2.418 min (gradient A), LC-MS (M+H)=590. The compound of Example 11: RT=2.339 min (gradient A), LC-MS (M+H)=590. The compound of Example 12: RT=2.502 min (gradient A), LC-MS (M+H)=590. The compound of Example 13: RT=2.449 min (gradient A), LC-MS (M+H)=590.

By the general procedures set forth above, the following compounds were also made.

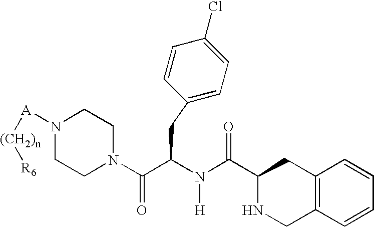

| Example | —A—(CH₂)ₙ—R₆ | MW | MS ion | Retention |
|---|---|---|---|---|
| 10 | 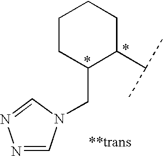 | 590.2 | 590 | 2.418 |
| 11 | 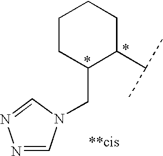 | 590.2 | 590 | 2.339 |
| 12 | 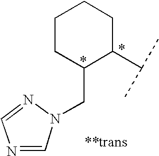 | 590.2 | 590 | 2.191 |
| 13 | 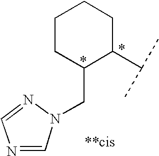 | 590.2 | 590 | 2.168 |

### Example 14

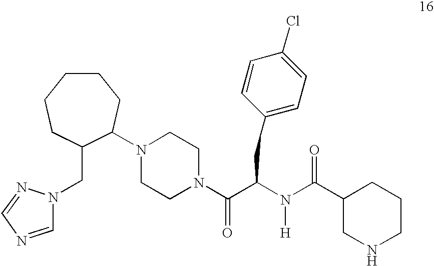

Step 14A: Synthesis of Keto-Triazoles 37 and 38

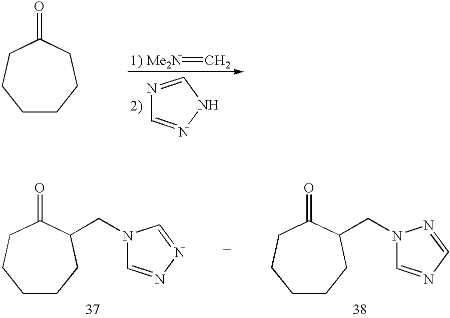

Cycloheptanone (2.60 mL, 22.0 mmol) and dimethyl methyleneammonium chloride (1.87 g, 20.0 mmol) were suspended in acetonitrile (10 mL) and heated in a sealed tube at 100° C. for 1 h. The mixture was cooled and the resulting solid isolated by filtration (1.82 g). This material was combined with triazole (1.83 g,26.5 mmol) and heated to reflux in 1:1 ethanol-water (20 mL) for 4 h. The mixture was concentrated under vacuum, taken up in dichloromethane, washed with aqueous sodium chloride, dried (MgSO₄) and again concentrated. The residue was purified by flash chromatography (elution with 2-5% methanol in dichloromethane) to afford 337 mg (9%) of 37 as a colorless oil: ¹H-NMR (300 MHz) δ 8.08 (s, 1H), 7.89 (s, 1H), 4.54 (dd, J=13.7, 8.0 Hz, 1H), 4.09 (dd, J=13.5, 5.7 Hz, 1H), 3.38-3.28 (m, 1H), 2.45-3.40 (m, 2H), 1.98-1.45 (m, 6H), 1.37-1.20 (m, 2H); LCMS 194 (MH⁺). Compound 38 was recovered as a white powder: mp 80-82° C.; ¹H-NMR (300 MHz) δ 8.17 (s, 2H), 4.39 (dd, J=14.1, 7.8 Hz, 1H), 4.02 (dd, J=14.1, 4.8 Hz, 1H), 3.06-2.97 (m, 1H),2.55-2.37 (m, 2H),1.97-1.76 (m, 3H),1.73-1.47 (m, 3H),1.42-1.23 (m, 2H); LCMS 194 (MH⁺).

Step 14B: Reductive Amination

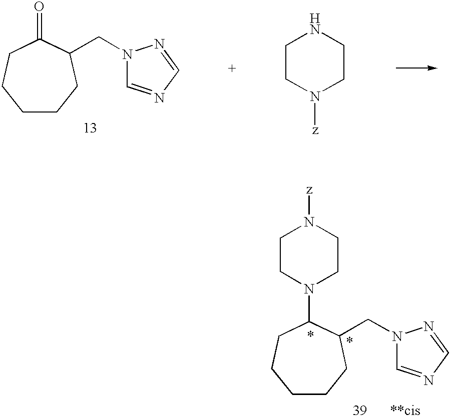

Ketone 38 (100 mg, 0.52 mmol) and benzyl 1-piperazinecarboxylate (0.32 mL, 1.66 mmol) were dissolved in dichloromethane (6 mL) and cooled to 0° C. A 1.0 M solution of titanium(IV) chloride in dichloromethane (0.52 nL, 0.52 mmol) was added and the mixture was stirred at 0° C. for 30 minutes and for 3 hours at room temperature. A solution of sodium cyanoborohydride (111 mg, 1.77 mmol) in isopropanol (6 mL) was added and stirring was continued for 20 h. Water (1 mL) was added and the mixture was stirred for 5 min. and filtered. The filtrate was concentrated and the residue was purified by preparative TLC to afford 15 mg (7%) of compound 39 as a colorless oil: ¹H-NMR (300 MHz) δ 8.01 (s, 1H), 7.92 (s, 1H), 7.37-7.25 (m, 5H), 5.13 (s, 2H), 4.49 (dd, J=13.1, 3.5 Hz, 1H), 4.13 (dd, J=13.4, 7.7 Hz, 1H), 3.55-3.42 (m, 5H), 2.70-2.62 (m, 2H), 2.36-2.21 (m, 314), 2.13-2.11 (m, 1H), 1.74-1.70 (m, 2H), 1.53-1.25 (m, 8H); LCMS 398 (MH⁺).

Step 14C: Amide Bond Formation and Deprotection

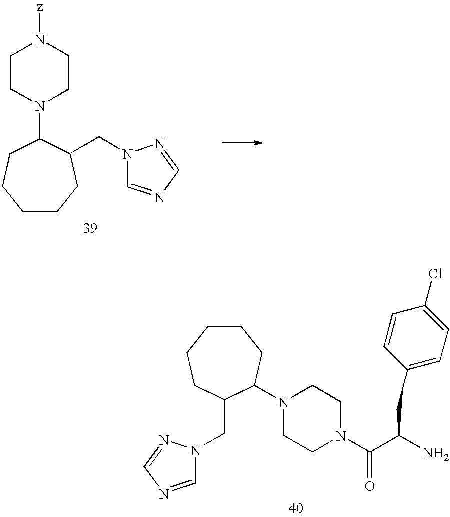

Triazole 39 (540 mg, 1.49 mmol), ammonium formate (500 mg, 8.0 mmol) and 10% palladium on charcoal (500 mg) were combined in ethanol (15 mL) and heated at 80° C. in a sealed tube for 10 min. The mixture was cooled and filtered (Celite). The solution was then concentrated under vacuum. For compounds protected with a butyloxycarbonyl (boc), this group was removed by dissolving the material in dichloromethane, adding an equal volume of TFA, and stirring at rt for 45 min. Concentration under vacuum afforded the TFA salt of the deprotected amine, which was used directly in subsequent steps.

The residue from above was dissolved in dichloromethane (15 mL) and treated with triethylamine (1.0 mL, 7.4 mmol), boc-D-phe(4-Cl)—OH (445 mg, 1.49 mmol) and HOBt (221 mg, 1.63 mmol). The mixture was stirred for 10 min and treated with EDC (313 mg, 1.63 mmol). It was stirred for 20 h, washed with aqueous sodium bicarbonate, dried (MgSO₄) and concentrated-upper vacuum. The residue was purified by flash chromatography (elution with ethyl acetate) to afford 218 mg (27%) of the desired amide: LCMS (MH⁺, 545). This material was dissolved in DCM, treated with TFA (15 mL) and stirred for 45 min. The mixture was concentrated under vacuum to afford 40 as a pale yellow oil.

Step 14D: Amide Bond Formation and Deprotection

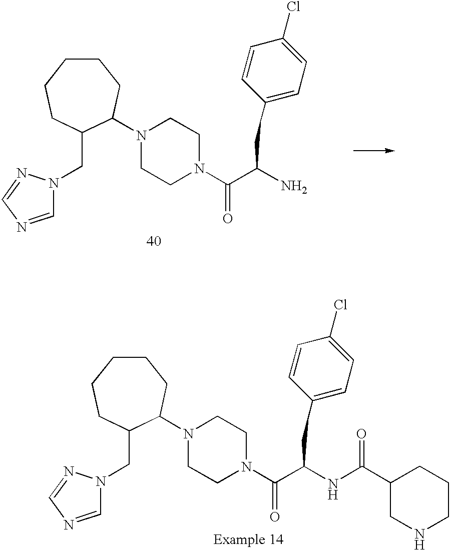

Example 14 was prepared from 40 and boc-protected nipecotic acid using the same procedure as used in the conversion of 39 to 40 in Step 14C. Example 14: LCMS (t_{R}, 2.188 (gradient A)) 556 (MH⁺).

By the general procedures set forth above, the following compounds were also made.

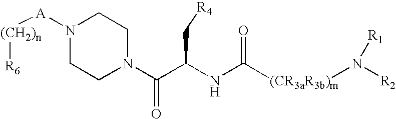

| Example | —A—(CH₂)ₙ—R₆ | R₄ | 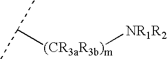 | MW | MS ion | Retention |
|---|---|---|---|---|---|---|
| 14-1 | 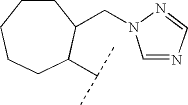 | 4-Cl-Ph | 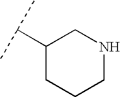 | 556.2 | 556 | 2.188 |
| 14-2 | 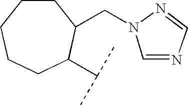 | 4-Cl-Ph | 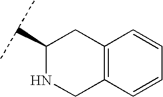 | 604.2 | 604 | 2.458 |
| 14-3 | 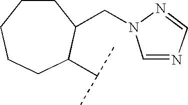 | 4-Cl-Ph | —CH₂CH₂NH₂ | 516.1 | 516 | 2.405 |
| 14-4 | 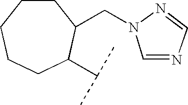 | 4-Cl-Ph | —CH₂NH₂ | 502.1 | 502 | 2.157 |
| 14-5 | 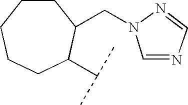 | 4-Cl-Ph | —CH₂CH₂CH₂NH₂ | 530.1 | 530 | 2.117 |
| 14-6 | 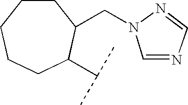 | 4-Cl-Ph | —CH₃ | 487.0 | 487 | 2.301 |
| 14-7 | 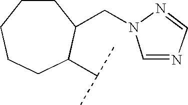 | 3,4-di-Cl-Ph | —CH₂CH₂NH₂ | 550.5 | 550 | 2.191 |
| 14-8 | 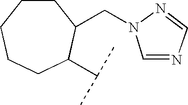 | 2,4-di-Cl-Ph | —CH₂CH₂NH₂ | 550.5 | 550 | 2.194 |
| 14-9 | 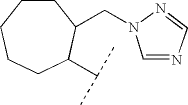 | Ph | —CH₂CH₂NH₂ | 481.6 | 482 | 2.048 |
| 14-10 | 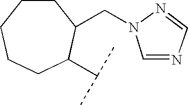 | 4-Cl-Ph | 3-Pyridyl | 550.1 | 550 | 2.261 |
| 14-11 | 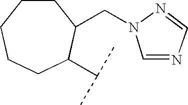 | 4-Cl-Ph | 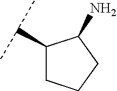 | 556.2 | 556 | 2.222 |
| 14-12 | 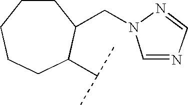 | 4-Cl-Ph | 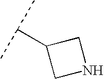 | 528.1 | 528 | 2.195 |
| 14-13 | 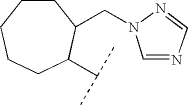 | 4-Cl-Ph | 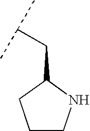 | 556.2 | 556 | 2.189 |
| 14-14 | 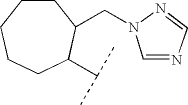 | 3,4-di-Cl-Ph | 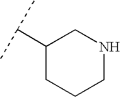 | 590.6 | 590 | 2.23 |
| 14-15 | 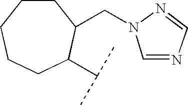 | 2,4-di-Cl-Ph | 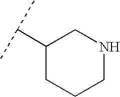 | 590.6 | 590 | 2.225 |
| 14-16 | 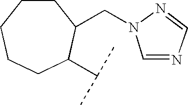 | Ph | 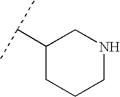 | 521.7 | 522 | 2.231 |
| 14-17 | 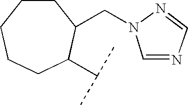 | 4-Cl-Ph | 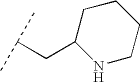 | 570.2 | 570 | 2.269 |
| 14-18 | 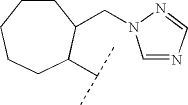 | 4-Cl-Ph | 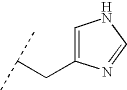 | 553.1 | 553 | 2.167 |
| 14-19 | 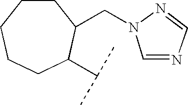 | 4-Cl-Ph | 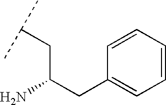 | 606.2 | 606 | 2.269 |
| 14-20 | 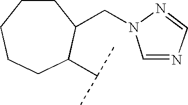 | 4-Cl-Ph | 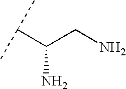 | 531.1 | 531 | 2.096 |
| 14-21 | 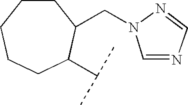 | 4-Cl-Ph | 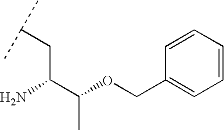 | 650.3 | 650 | 2.335 |
| 14-22 | 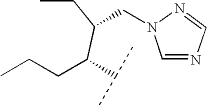 | 4-Cl-Ph | 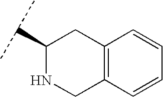 | 606.2 | 606 | 2.478 |
| 14-23 | 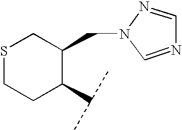 | 4-Cl-Ph | 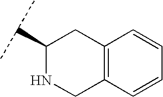 | 608.2 | 608 | 2.392 |
| 14-24 | 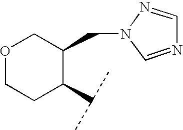 | 4-Cl-Ph | 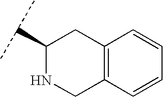 | 592.1 | 592 | 2.346 |
| 14-25 | 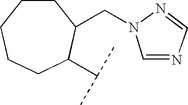 | 4-Cl-Ph | 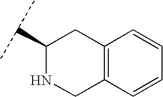 | 604.2 | 604 | 2.43 |

### Example 15

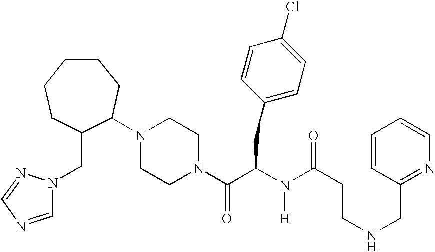

Step 15A: Formation of Acrylamide 41

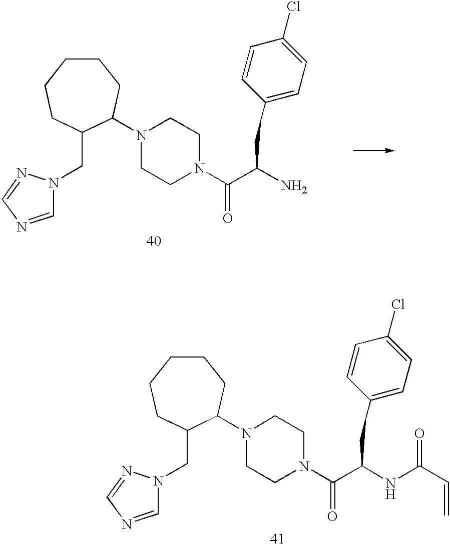

Compound 40 (0.37 mmol) was dissolved in DCM (5 mL), treated with TEA (0.26 mL) and cooled to 0° C. Acryloyl chloride (0.036 mL, 0.44 mmol) was added, the ice-bath was removed, and stirring was continued for 20 h. The mixture was poured into aqueous sodium bicarbonate and extracted with DCM. The combined extracts were dried (MgSO₄) and concentrated to afford 169 mg of crude 41 as a white foam: LCMS (MH⁺, 499).

Step 15B: Addition of 2-(aminomethyl)pyridine to 41

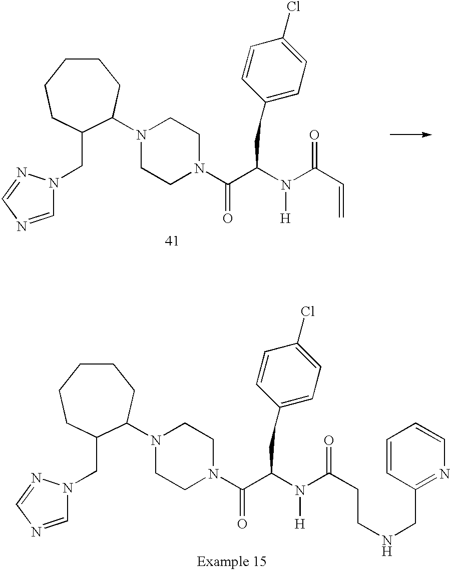

Acrylamide 22 (20 mg, 0.040 mmol) was dissolved in methanol (1 mL), 2-(aminomethyl)pyridine (2 drops) was added, and the mixture was heated at 80° C. in a sealed vial for 20 h. The mixture was cooled to rt, and purified directly by preparative HPLC to 5 afford Example 15 as a colorless oil: LCMS (tR 2.215 min. (gradient A); MH+607.

By the general procedures set forth above, the following compounds were also made.

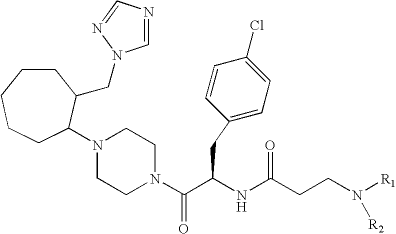

| Example | —NR₁R₂ | MW | MS ion | Retention |
|---|---|---|---|---|
| 15-1 | 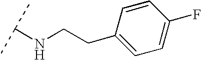 | 638.2 | 638 | 2.315 |
| 15-2 | 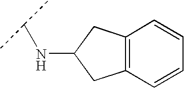 | 632.2 | 632 | 2.3 |
| 15-3 | 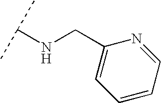 | 607.2 | 607 | 2.215 |
| 15-4 | 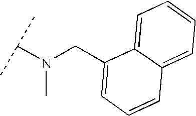 | 670.3 | 670 | 2.369 |
| 15-5 | 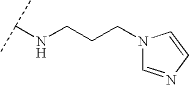 | 624.2 | 624 | 2.348 |
| 15-6 | 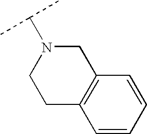 | 632.2 | 632 | 2.504 |

### Example 16

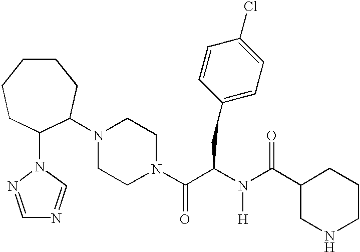

Step 16A: Synthesis of Keto-Triazole 42

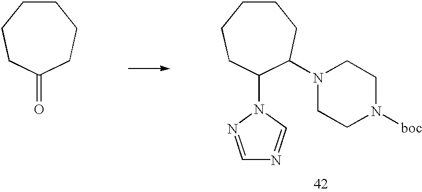

Cycloheptanone (5.30 mL, 47.7 mmol) was dissolved in acetic acid (5 mL) and water (7 mL) and warmed to 60° C. Bromine (2.20 mL, 42.9 mmol) was added over 10 min. Heating was continued for 40 min., the mixture was cooled to rt, and potassium carbonate (10 g) was cautiously added. The mixture was poured into water, extracted with DCM, and the combined extracts were dried (MgSO₄) and concentrated. The residue was combined with 1,2,4-triazole (3.42 g, 49.5 mmol) and potassium carbonate (9.24 g, 66.9 mmol) in acetone (200 mL), and the mixture was heated at 60° C. for 20 h. The mixture was filtered, concentrated, taken up in DCM, washed with aqueous sodium chloride, dried (MgSO₄), and again concentrated. The residue was crystallized from ether to afford 1.70 g (20%) of 42 as a white powder: LCMS (MH⁺, 180).

Step 16B

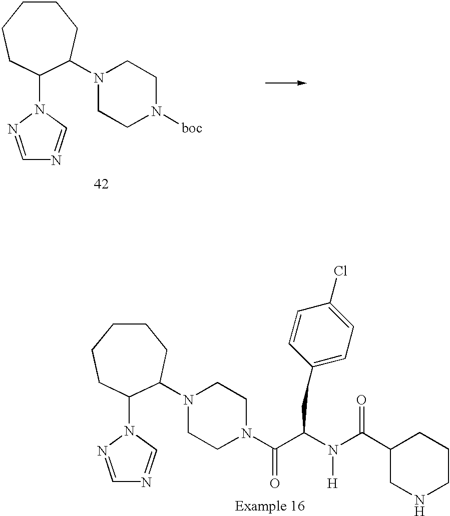

Triazole 42 was elaborated into Example 16 in the same manner as in the conversion of compound 39 into Example 14 as shown in Steps 14c and 14d. Example 16: LCMS (t_{R}, 2.433 (gradient A)) 542 (MH⁺).

By the general procedures set forth above, the following compounds were also made.

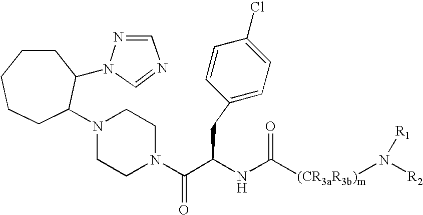

| Example | —(CR₃ₐR_{3b})ₘ—NR₁R₂ | MW | MS ion | Retention |
|---|---|---|---|---|
| 16-1 | —CH₂CH₂NH₂ | 502.1 | 502 | 2.41 |
| 16-2 | 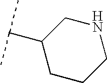 | 542.1 | 542 | 2.433 |
| 16-3 | 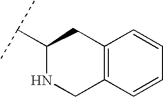 | 590.2 | 590 | 2.478 |

### Example 17

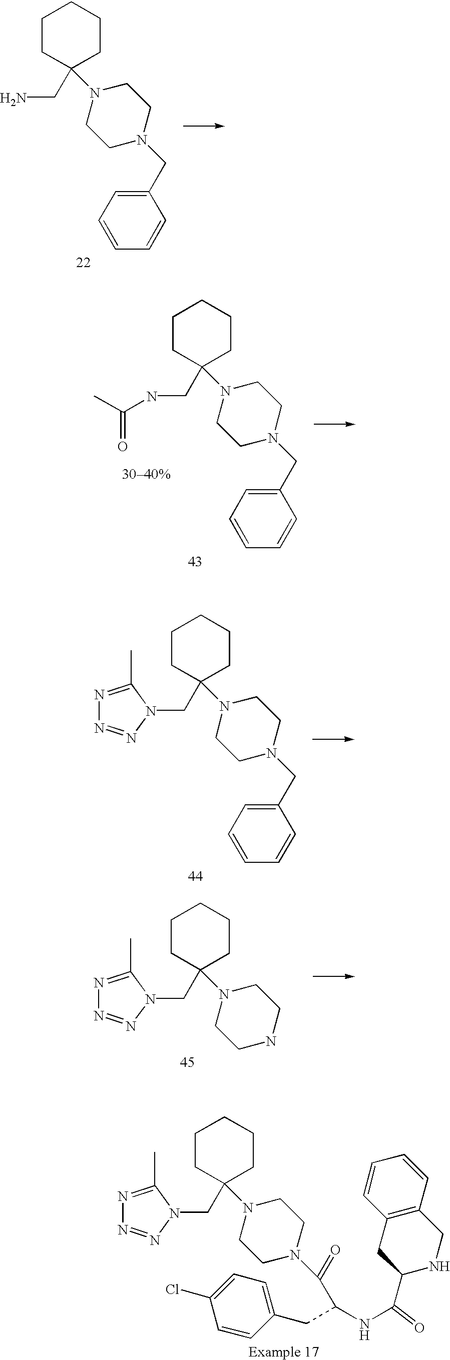

Step 17A

To a mixture of 4 mL acetic anhydride and 0.5 mL TEA was added Compound 22 (see example 6, M.W. 287, 1.4 mmol, 0.4 g). The reaction mixture was stirred at RT overnight. The reaction mixture was concentrated and purified by preparative TLC plates (4 plates), using a mixture of CHCl₃, MeOH, ethyl acetate and aminonium hydroxide. Compound 43 was purified by preparative thin layer chromatography and isolated as an oil. ¹H NMR (CDCl₃), δ=1.19-1.82 (m, 10H), 2.00 (s, 3H), 2.71 (m, 4H), 2.91 (m, 4H), 3.43(d, 2H), 3.68 (s, 2H), 7.24-7.32 (m, 5H, aromatic).

Step 17B

In 5 mL dry acetonitrile were added 43 (80 mg, 0.24 mmol), trifluorosulfonyl anhydride (0.08 g, 1.2 eq), and sodium azide (0.02 g, 1.2 eq). The reaction mixture was stirred overnight. The reaction mixture was extracted by 5 mL CH₂Cl₂ and 5 mL saturated NaHCO₃, dried over Na₂SO₄ and concentrated to give 44.

Step 17C

To 10 mL ethanol was added the crude 44 and 0.6 g ammonium formate followed by 0.2 g Pd (20%W on carbon). The mixture was sealed and heated at 80° C. for 2 hours. The mixture was filtered through celite and concentrated to give 40 mg (62% two steps) 45. ¹H NMR (CDCl₃), δ=1.2-1.8 (m, 10H), 2.59 (s, 2H), 2.89-3.31 (m, 8H), 3.86 (s, 3H).

Step 17D

Coupling of 45 to the D-pCl-Phe-D-Tic-Boc dipeptide, deprotection and HPLC purification as described previously in Steps 14c and 14d provided Example 17.

### Example 18

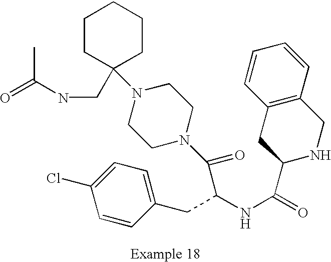

To 10 mL ethanol was added 43 and 0.6 g ammonium formate followed by 0.2 g Pd (20%W on carbon). The mixture was sealed and heated at 80° C. for 2 hours. The mixture was filtered through celite and concentrated to give 40 mg of deprotected intermediate. ¹H NMR (CDCl₃), δ=1.2-1.8 (m, 10H), 2.01 (s, 3H), 3.40 (d, 2H), 3.44-3.55 (m, 8H). Coupling of this intermediate to the D-pCl-Phe-D-Tic-Boc dipeptide, deprotection and HPLC purification as described previously provided Example 18.

### Example 19

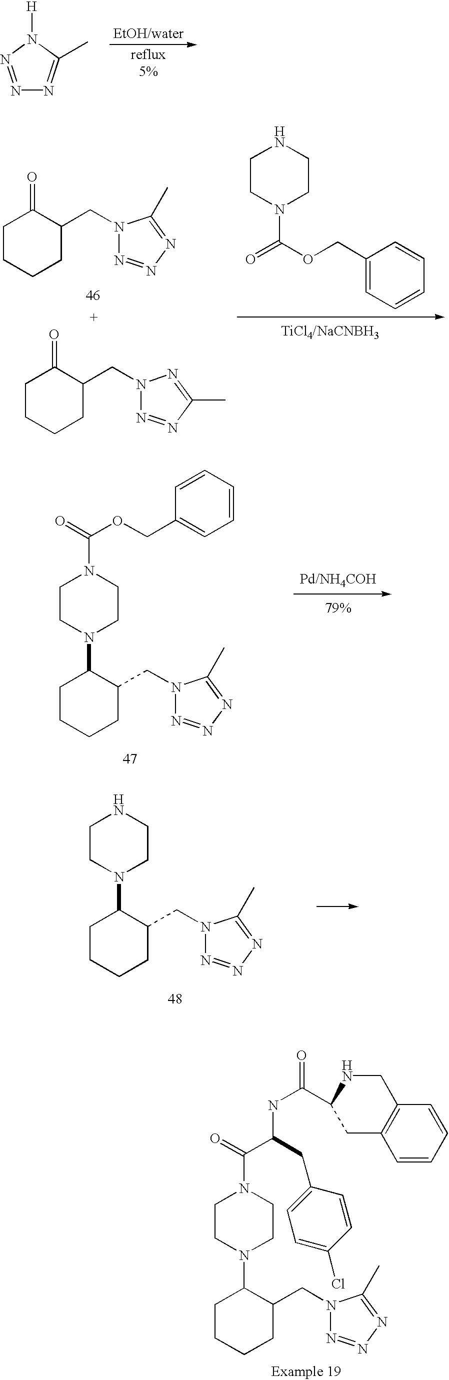

Step 19A

In a mixture of 10 mL/10 mL water/EtOH were added 2-(dimethylaminomethyl)-1-cyclohexanone (1.5 g,7.8 mmol) and methyl-tetrazole (2.6 g, 31.2 mmol, 4 eq). The reaction mixture was refluxed for 6 hours. The reaction mixture was dried, extracted with 20 mL brine and 20 mL CH₂Cl₂, the organic layer dried over Na₂SO₄, concentrated, and purified by Jones column (10 g, 0-80% ethyl acetate in hexane in 23 mins). Obtained compound 46 as a clear oil. 1I NMR (300 MHz, CDCl₃), δ=1.43-1.48 (m, 1H), 1.66-1.70 (m, 3H), 1.88-1.90 (m, 3H), 2.11-2.20 (m, 1H), 2.52(s, 3H), 3.11-3.12 (m, 1H), 4.42-4.49 (dd, 1H), 4.95-5.02 (dd, 1H).

Step 19B

In 10 mL CH₂Cl₂ at 0° C. were added 46 (0.11 g, M.W. 194, 0.57 mmol) and Cbz piperazine (0.35 mL, 1.6 mmol, 2.5 eq), followed by TiCl₄ (0.6 mL, 1.0M solution). The reaction mixture was stirred at 0° C. for 30 mins, then 2 hours at room temperature. A solution of NaCN-BH₃ in isopropanol (0.14 g in 7 mL) was added and the reaction mixture was stirred at room temperature overnight. The reaction mixture was concentrated and loaded directly onto 4 prep-TLC plates. The plates were eluted by 850/150/2 CHCl₃/MeOH/NH₃, the appropriate band cut and eluted, concentrated to obtain 140 mg of 47 as a clear oil. ¹H NMR (300 MHz, CDCl₃), δ=1.25-2.59 (m, 16H), 2.52 (s, 3H), 2.73 (m, 1H), 4.63 (dd, 1H), 4.78 (dd, 1H), 5.14 (s, 2H), 7.36(s, 5H).

Step 19C

To 5 mL EtOH were added 47 (130 mg, M.W. 398, 0.33 mmol), ammonium formate (200 mg) and 50 mg Pd (10% on carbon). The mixture was headed at 80° C. for 1 hour. The mixture was filtered through a 50 micro A disc and concentrated to give 68 mg of 48 as a clear oil.

Step 19D

Coupling of 48 to the D-p-Cl-Phe-D-Tic-Boc dipeptide, deprotection and HPLC purification as described previously provided Example 19.

### Example 20

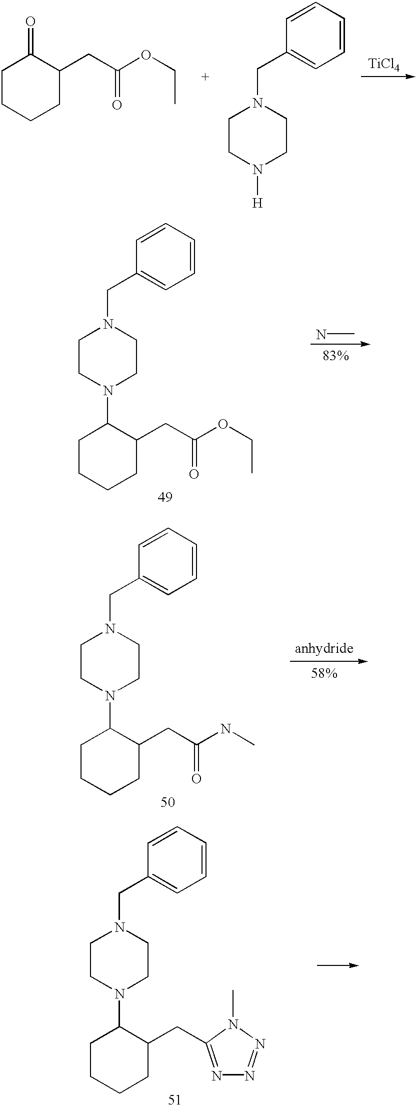

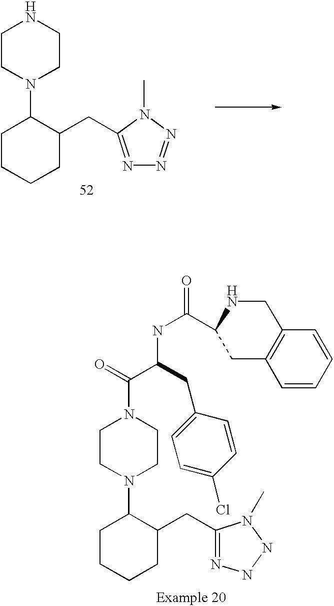

Step 20A

Compound 49 was obtained by the procedure as Step 19B using benzylpiperazine and ethyl 2-cyclohexanoneacetate as starting materials. ¹H NMR (300 MHz, CDCl₃), δ=1.21-1.26 (t, 3H), 1.13-2.61 (m, 19H), 3.49 (s, 2H), 4.05-4.12 (q, 2H), 7.29 (m, 5H).

Step 20B

In 10 mL 1.0M methylamine in MeOH were added NaOMe and compound 49 (0.3 g, M.W. 344, 0.87 mmol). The reaction mixture was sealed and heated at 70° C. for two days. The reaction mixture was concentrated and purified by three prep-TLC plates, using 95/5 CH₂Cl₂/MeOH. Compound 50 was obtained as a white solid (240 mg, 83.6% yield).

¹H NMR (300 MHz, CDCl₃), δ=1.19-2.76 (m, 18H), 2.95-2.96 (d, 2H), 3.3.50-3.51 (d, 3H), 5.29 (s, 2H), 7.29 (m, 5H).

Step 20C

In 4 mL of CH₃CN were added NaN₃ (30 mg, 65,0.3 1 mmol), (CF₃SO₂)₂O (82 mg, 0.3 mmol) and 50 (80 mg, M.W. 329, 0.24 mmol). The reaction mixture was stirred at room temperature overnight. LC-MS showed 60% reaction, additional 50 mgNaN₃ and 100 μL anhydride were added and the reaction was stirred for another day. The reaction was purified by LC-MS, giving 50 mg of compound 51 (58% yield).

Deprotection of 51, and coupling with dipeptide, followed by Boc deprotection and HPLC purification as previously described provided Example 20 (T_{R} 2.45, MS 605).

### Example 21

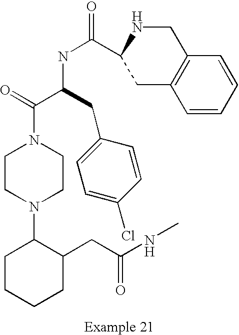

Transfer catalysis hydrogenation mediated benzyl deprotection of amide 50, coupling to the corresponding dipeptide, Boc-deprotection and HPLC purification as previously described produced Example 21 (TR 2.43, MS 580).

### Example 22

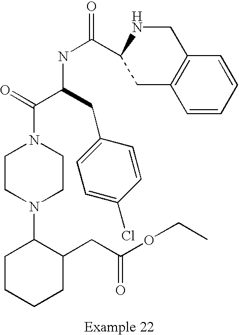

Transfer catalysis hydrogenation mediated benzyl deprotection of ester 49, coupling to the corresponding dipeptide, Boc-deprotection and HPLC purification as previously described produced Example 22 (T_{R} 2.55, MS 595).

### Example 23

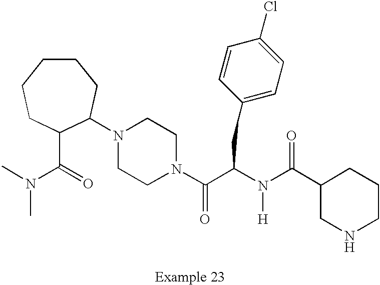

Step 23A: Synthesis of Methyl Ester 53

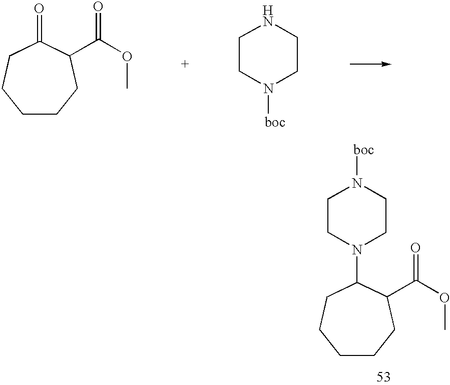

Compound 53 was prepared from 2-(methoxycarbonyl)cycloheptanone using the procedure of Step 14B. Compound 53: LCMS 341 (MH⁺).

Step 23B: Saponification of Methyl Ester

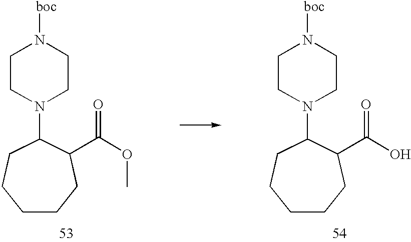

The methyl ester (500 mg, 1.47 mmol) was dissolved in 4 mL of 1,4-dioxane and a solution of lithium hydroxide (617 mg, 14.7 mmol in 0.5 mL of water) was added. This mixture was heated at reflux overnight. The reaction was cooled, concentrated, dissolved in dichloromethane and washed with 5% citric acid. The organic layer was dried (Na₂SO4) and evaporated to afford 380 mg (80%) of 54: LCMS 327 (MH⁺).

Step 23C: Synthesis of Compound 55

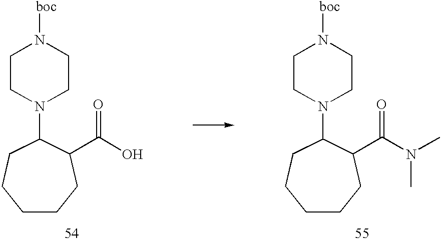

Carboxylic acid 54 (25 mg, 0.080 mmol) was dissolved in dichloromethane. TEA (0.022 ml, 0.16 mmol), dimethylamine (0.08 mmoles), and HOBt (12 mg, 0.088 mmol) were added and the solution was stirred for 10 min. EDC (17 mg, 0.088 mmol) was added and the reaction was stirred overnight and was partitioned between dichloromethane and saturated sodium bicarbonate. The organic layer was then washed with saturated sodium chloride solution, dried (Na₂SO₄), and evaporated. The crude material was used without further purification. Compound 55: LCMS 354 (MH⁺).

Step 23D: Synthesis of Example 23

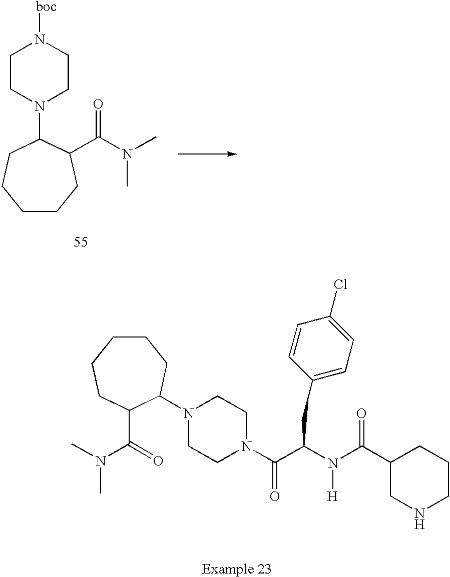

Example 23 was prepared from 55 using the same procedure shown in Step 14C and Step 14D. Example 23: LCMS (t_{R}, 2.180 (gradient A)) 546 (MH⁺).

By the general procedures set forth above, the following compounds were also made.

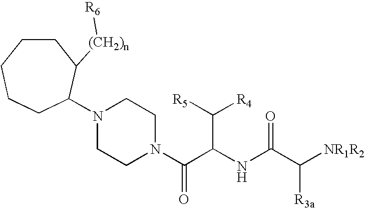

| Example | —(CH₂)ₙR₆ | —CHR₄R₅ | —CHR₃ₐNR₁R₂ | MW | MS ion | Retention |
|---|---|---|---|---|---|---|
| 23-1 | —C(O)N(CH₃)₂ | 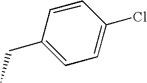 | —CH₂CH₂NH₂ | 506.1 | 506 | 2.156 |
| 23-2 | —C(O)N(CH₃)₂ | 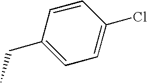 | 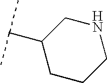 | 546.2 | 546 | 2.18 |
| 23-3 | —C(O)NH(CH₃) | 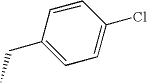 | 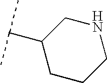 | 532.1 | 532 | 2.136 |
| 23-4 | —CO₂CH₃ | 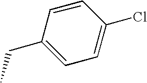 | —CH₂CH₂NH₂ | 493.0 | 493 | 2.223 |
| 23-5 | —CO₂NHBz | 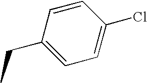 | 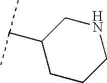 | 608.2 | 608 | 2.317 |
| 23-6 | —C(O)NH(CH₂)₂—(2-imidazole) | 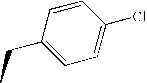 | 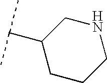 | 612.2 | 612 | 2.064 |
| 23-7 | —C(O)NH(CH₂)₂—(4-F-Ph) | 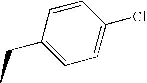 | 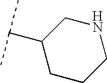 | 640.2 | 640 | 2.376 |
| 23-8 | —C(O)NH(CH₂)₂—N(CH₃)₂ | 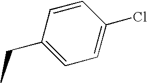 | 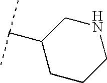 | 589.2 | 589 | 2.066 |
| 23-9 | —CO₂CH₃ | 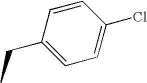 | 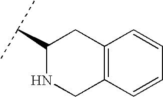 | 581.2 | 581 | 2.518 |
| 23-10 | —CO₂CH₃ | 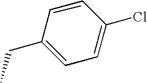 | —CH₂CH₂NH—C(NH)NH₂ | 535.1 | 535 | 2.246 |
| 23-11 | —CO₂CH₃ | 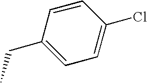 | 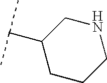 | 533.1 | 533 | 2.199 |

### Example 24

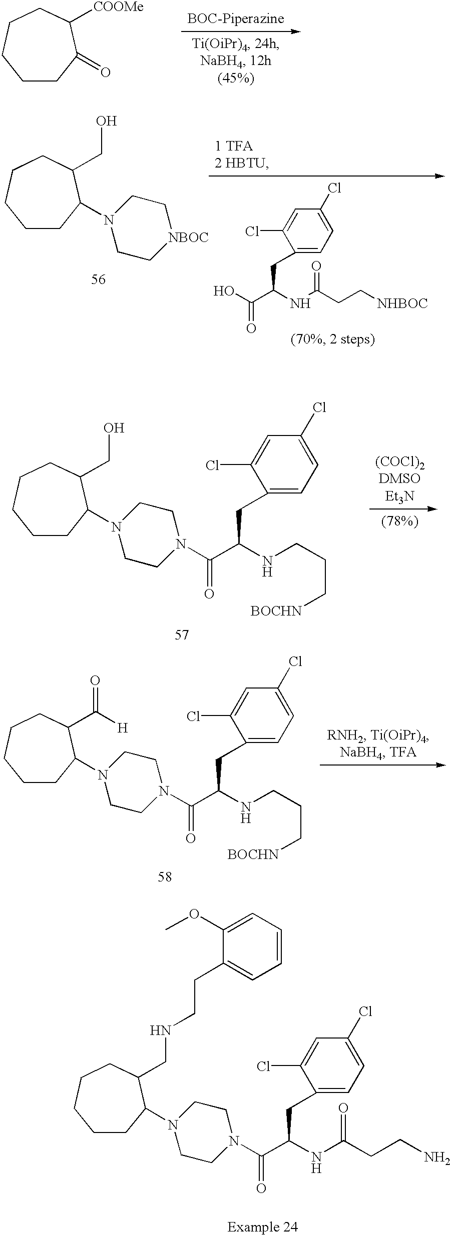

Step 24A:

To a stirring solution of 2-oxocycloheptanecarboxylic acid methyl ester (2.30 g, 13.5 mmol) and BOC-piperazine (5.0 g, 27 mmol) in dry ethanol (20 mL) under nitrogen was added titanium (IV) isoproproxide (8.0 mL, 27 mmol), and stirring was continued for 24 h. Sodium borohydride (1.5 g, 41 mmol) was then added, and the resulting suspension was stirred overnight. The mixture was diluted with ethyl acetate (60 mL) and quenched with 2N aq. ammonium hydroxide (40 mL), then filtered over celite, rinsing with ethyl acetate. The layers were separated, and the aqueous extracted with ethyl acetate (3×50 mL). The combined organics were dried (magnesium sulfate), concentrated, and purified by column chromatography (99:1 dichloromethane: triethylamine to 96:3:1 dichloromethane:methanol:triethylamine) to give the 1-(tertiary-butoxycarbonyl)-4-{2-(hydroxymethyl)cycloheptyl}piperazine 56 as a viscous, colorless oil (1.91 g,45%), MS (MH⁺) 313.2.

Step 24B:

To 1-(tertary-butoxycarbonyl)-4-{2-(hydroxymethyl)cycloheptyl}piperazine 56 (1.72 g, 5.51 mmol) in dichloromethane (5 mL) was added TFA (5 mL) and stirring was continued for 30 min. Concentration, followed by addition of 1:1 dichloromethane: diisopropylethylamine (10 mL), and subsequent re-concentration gave the free base as a paste. A solution of the dipeptide N-Boc-b-Alanine-(2,4-Cl)-phenylalanine (2.45 g, 6.06 mmol) and HBTU (2.30 g, 6.06 mmol) in DMF (8 mL) was stirred for 30 min, then added to the free base. Stirring was continued overnight, then the solution was diluted with ethyl acetate (100 mL) and washed with sat. aq. sodium bicarbonate (100 mL). The aqueous layer was extracted with ethyl acetate (3×100 mL), and the combined organics were washed with brine (100 mL), dried (magnesium sulfate), concentrated and purified by column chromatography (95:5 dichloromethane:methanol) to give 3-Boc-amino-N-[1-(2,4-dichlorobenzyl)-2-oxo-2-(4-{2-[hydroxymethyl]cycloheptyl}piperazin-1-yl)ethyl]propionamide57 as apaleyellowoil (2.63 g, 70%). MS (MH⁺) 599.2.

Step 24C:

To oxalyl chloride (0.66 g, 5.2 mmol) in dichloromethane (10 mL) at −78° C. was added dropwise DMSO (0.65 mL, 9.2 mmol) and the mixture was stirred for 30 min. A solution of 3-Boc-amino-N-[ ]-(2,4-dichlorobenzyl)-2-oxo-2-(4-{2-[hydroxymethyl]cycloheptyl}piperazin-1-yl)ethyl]propionamide 57 (2.25 g, 3.76 mmol) in dichloromethane (10 mL) was added via canula, and stirting was continued for 1 h. Triethylamine (2.6 mL, 18.8 mmol) was then added dropwise, and the mixture was stirred at −78° C. for 1 h, then allowed to warm to ambient temperature over 20 min. The mixture was quenched with sat. aq. sodium bicarbonate (10 mL) and separated, and the aqueous extracted with dichloromethane (2×20 mL). The combined organics were washed with brine (50 mL), dried (magnesium sulfate), concentrated and purified by column chromatography (96:4 dichloromethane:methanol) to give 3-Boc-amino-N-[1-(2,4-dichlorobenzyl)-2-oxo-2-(4-{2-formylcycloheptyl}piperazin-1-yl)ethyl]propionamide 58 as apale yellow foam (1.76 g, 78%). MS (MH⁺) 597.2.

Step 24D: 3-Amino-N-[1-(2,4-dichlorobenzyl)-2-oxo-2-(4-{2-[2-(2-methoxyphenethylamino)methyl]cycloheptyl}piperazin-1-yl)ethyl]propionamide

To 3-Boc-amino-N-[1-(2,4-dichlorobenzyl)-2-oxo-2-(4-{2-formylcycloheptyl}piperazin-1-yl)ethyl]propionamide 58 (100 mg, 0.167 mmol) and 2-methoxyphenethylamine (50 mg, 0.334 mml, 2 eq.) in dry ethanol (1 mL) was added added titanium (IV) isoproproxide (100 μL, 0.251 mmol), and stirring was continued for 24 h. Sodium borohydride (9.5 mg, 0.25 mmol) was then added, and the resulting suspension was stirred overnight. The mixture was evaporated, diluted with ethyl acetate (1 mL) and quenched with 2N aqueous ammonium hydroxide (1 mL), then filtered over celite, rinsing with ethyl acetate. The layers were separated, and the combined organics were dried (magnesium sulfate) and concentrated. Dichloromethane (1 mL) and TFA (1 mL) were added and the mixture was stirred for 30 min. The mixture was evaporated and purified by preparative LCMS to give Example 24. (MH⁺=633)

By the general procedures set forth above, the following compounds were also made.

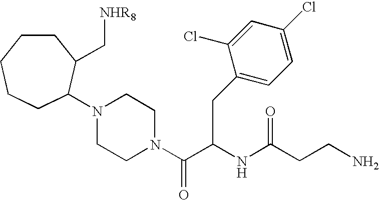

| Example | R₈ | MS (MH+) | MW |
|---|---|---|---|
| 24-1 | 2-(2-methoxyphenyl)ethyl | 633 | 632.7 |
| 24-2 | 1-methoxy-2-propyl | 571 | 570.6 |
| 24-3 | 2-(2-thiophenyl)ethyl | 609 | 608.7 |

### Example 25

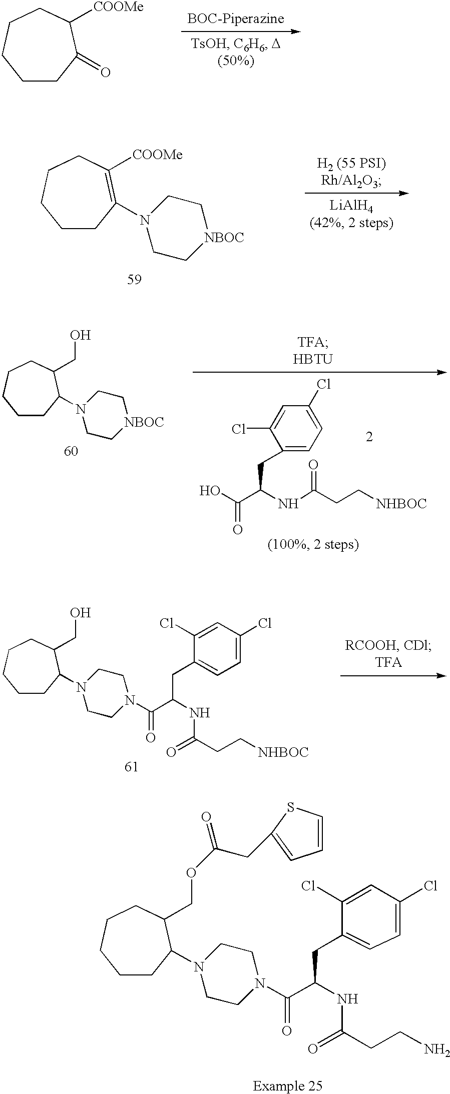

Step A:

A solution of 2-oxocyclobeptanecarboxylic acid methyl ester (3.00 g, 17.6 mmol), BOC-piperazine (1.86 g, 24.7 mmol) and toluenesulfonic acid (70 mg, 0.35 mmol) in dry benzene (20 mL) was refluxed using a Dean-Stark apparatus under nitrogen for 48 h. The mixture was concentrated and filtered over silica gel (eluting with 70:30 dichloromethane: ethyl acetate) to give the crude enamine 59 as a viscous, yellow oil (3.0 g, 50%), which was used directly in the next step. The enamine 59 was dissolved in 50 mL dry methanol, and 5% rhodium on alumina (850 mg) was added. The mixture was hydrogenated at 55 PSI for 40 h, filtered over celite and evaporated to give the crude ester as a white solid (2.65 g). The ester was immediately dissolved in 50 mL dry THF under nitrogen, cooled to 0° C., and solid LAH (0.90 g, 24 mmol) was added in portions. The mixture was then stirred at room temperature for 20 min., quenched with sat. aq. potassium carbonate (4.5 mL), filtered over celite, and dried over magnesium sulfate. Concentration, followed by purification by column chromatography (96:3:1 dichloromethane:methanol:triethylamine) to give 1-(tertary-butoxycarbonyl)-4-{2-(hydroxymethyl)cycloheptyl}piperazine 60 as aviscous, colorless oil (1.17 g, 42%). MS (MS⁺) 313.2.

Step B:

To 1-(tertary-butoxycarbonyl)-4-{2-(hydroxymethyl)cycloheptyl}piperazine 60 (0.750 g, 2.40 mmol) in dichloromethane (3 mL) was added TFA (2 mL) and stirring was continued for 20 min. Concentration, followed by addition of 1:1 dichloromethane: diisopropylethylamine (5 mL), and subsequent re-concentration gave the crude free base as a paste. A solution of the N-Boc-b-Alanine-(2,4-di-Cl)-phenylalanine (1.07 g, 2.64 mmol) and HBTU (0.910 g, 2.40 mmol) in DMF (4 mL) was stirred for 60 min, then added to the free base. Stirring was continued for 3 h, then the solution was diluted with ethyl acetate (100 mL) and washed with sat. aq. sodium bicarbonate (100 mL). The aqueous layer was extracted with ethyl acetate (3×100 mL), and the combined organics were washed with brine (100 mL), dried (magnesium sulfate), concentrated and purified by column chromatography (95:5 dichloromethane:methanol) to give 3-Boc-amino-N-[ ]-(2,4-dichlorobenzyl)-2-oxo-2-(4-{2-[hydroxymethyl]cycloheptyl}piperazin-1-yl)ethyl]propionamide 61 as a pale yellow oil (1.44 g, 100%). MS (MH⁺) 599.2.

Step C: 3-Amino-N-[1-(2,4-dichlorobenzyl)-2-oxo-2-(4-{2-[(2-{2-thiophenylmethyl}carboxy)methyl]cycloheptyl}piperazin-1-yl)ethyl]propionamide

To a solution of carbonyldiimidazole (17 mg, 0.10 mmol) in dichloromethane (0.5 mL) was added the 2-thiopheneacetic acid (14 mg,0.10 mmol). Stirring was continued for 10 min., then 3-Boc-amino-N-[1-(2,4-dichlorobenzyl)-2-oxo-2-(4-{2-[hydroxymethyl]cycloheptyl}piperazin-1-yl)ethyl]propionamide 61 (60 mg, 0.10 mmol) in 0.5 mL dichloromethane was added, and the mixture was stirred overnight. The mixture was then diluted with ethyl acetate (2 mL) and washed with sat. aq. sodium bicarbonate (1 mL). The organic layer was concentrated, then dichloromethane (1 mL) and TFA (1 mL) were added and the mixture was stirred for 30 min. The mixture was concentrated and purified by preparative LCMS to give Example 25 as viscous yellow oil. (MH⁺=624)

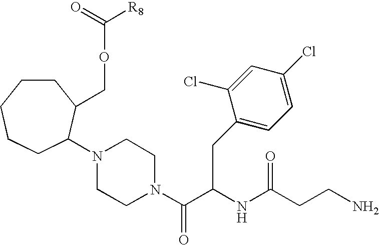

| Example | R₈ | MS (MH⁺) | MW |
|---|---|---|---|
| 25-1 | 2-thiophenylmethyl | 624 | 623.6 |
| 25-2 | 3-thiophenylmethyl | 624 | 623.6 |
| 25-3 | aminomethyl | 557 | 556.5 |
| 25-4 | ethylamino | 571 | 570.5 |

### Example 26

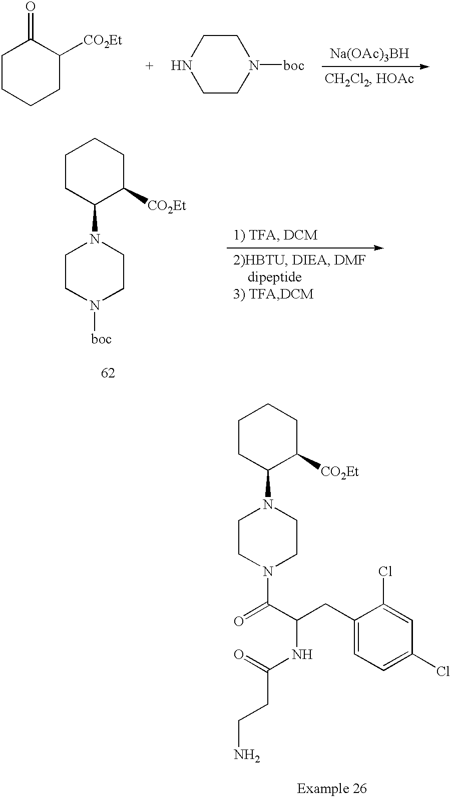

Step A. cis-4-(2-ethoxycarbonyl-cyclohexyl)-piperazine-1-carboxylic Acid Tert-Butyl Ester 62

A solution containing 2-oxo-cyclohexanecarboxylic acid ethyl ester (9.60 mL, 60.0 mmol), 1-Boc-piperazine (11.18 g, 60.0 mmol), HOAc (3.6 mL, 63.0 mmol) in dichloromethane (60 mL) was stirred at room temperature for 1.5 h. Sodium triacetoxy borohydride (31.79 g, 150.0 mmol) was added portionwise. The resulting white suspension was stirred vigorously at room temperature for 22 h. The reaction mixture was diluted with EtOAc (200 mL), and the organics were washed with H₂O, saturated NaHCO₃ and brine. After drying and concentration in vacuo, the resulting residue was chromatographed on silica-gel, eluting with a 4:1 v/v mixture of hexanes and EtOAc.

Compound 62 was isolated as a colorless oil. Yield: 5.45 g (16.0 mmol, 27%). LCMS m/z 341 (M⁺+1).

Step B: cis-2-{4-[2-(3-amino-propionylamino)-3-(R)-(2,4-dichloro-phenyl)-propion]-peperazin-1-yl}-cyclohexanecarboxylic Acid Ethyl Ester

cis-4-(2-Ethoxycarbonyl-cyclohexyl)-piperazine-1-carboxylic acid tert-butyl ester 62 (136 mg, 0.4 mmol) was dissolved in dichloromethane (2 mL) and to that solution, trifluoroacetic acid (1 mL) was added. The resulting solution was stirred at room temperature for 1 h. The reaction was deemed complete by TLC (4:1 v/v hexanes/EtOAc). The volatiles were removed in vacuo. The residue was then dissolved in DMF (1 mL) and treated with diisopropylethyl amine (140 μL, 0.80 mmol). This solution was set aside. In a separate flask, a solution containing the dipeptide (R)-2-(3-tert-butoxycarbonylamino-propionylamino)-3-(2,4-dichlorophenyl)-propionic acid (178 mg, 0.44 mmol) and diisopropylethyl amine (140 μL, 0.80 mmol) in DMF (2 mL), was treated with HBTU (200 mg, 0.52 mmol). The resulting golden yellow solution was stirred at room temperature, under N₂, for 30 minutes. The solution containing the deprotected amine was added to this, and the resulting mixture was stirred for 16 h at room temperature. The reaction was diluted with EtOAc (30 mL) and washed with 0.1 N HCl and then with saturated NaHCO₃. The organics were washed with brine, dried over anhydrous MgSO₄ and filtered. Evaporation gave a residue that was dissolved in dichloromethane (4 mL) and treated with trifluoroacetic acid (2 mL). After 2 h, the reaction was deemed complete by LCMS. The volatiles were removed under vacuum and the residue was purified by preparative HPLC/MS to give Example 26. Yield: 76 mg (0.14 mmol, 35%). LCMS m/z 527 (M⁺+1).

### Example 27

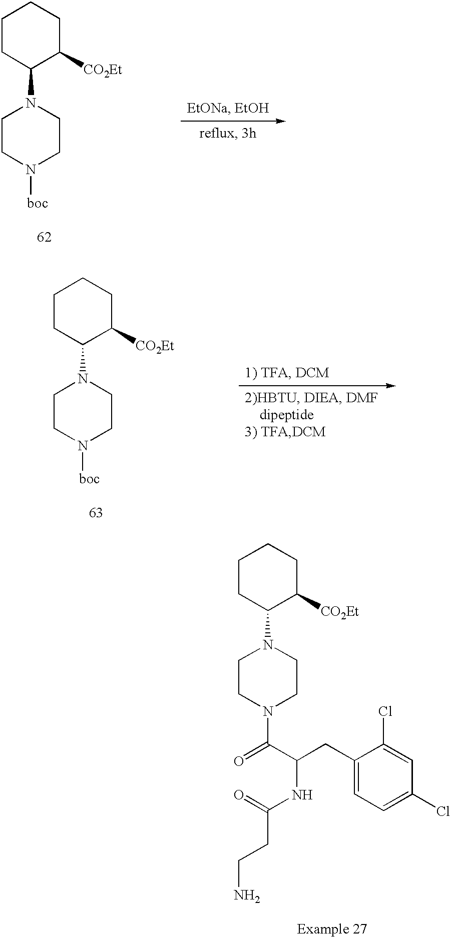

Step 27A. trans-4-(2-ethoxycarbonyl-cyclohexyl)-piperazine-1-carboxylic Acid Tert-Butyl Ester 63

Sodium metal (460 mg, 20.0 mmol) was cut into small pieces and added portionwise to EtOH (50 mL), under N₂. When all solids dissolved, compound 62 (3.40 g, 10.0 mmol) was added and the resulting mixture was refluxed for 3 h. The reaction mixture was cooled, diluted with EtOAc (100 mL) and washed with H₂O. The organics were washed with brine, dried over anhydrous MgSO₄ and filtered. Concentration under vacuum gave a yellow oil that was purified by column chromatography (eluting with a 9:1 v/v mixture of hexanes and EtOAc) to give compound 63 as a thick yellow oil that solidified upon standing (1.60 g, 4.7 mmol, 47%). LCMS m/z 341 (M⁺+1).

Step 27B: trans-2-{4-[2-(3-Amino-propionylamino)-3-(R)-(2,4-dichloro-phenyl)-propionyl]-piperazin-1-yl}-cyclohexanecarboxylic Acid Ethyl Ester Example 27

trans-4-(2-ethoxycarbonyl-cyclohexyl)-piperazine-1-carboxylic acid tert-butyl ester 63 (136 mg, 0.4 mmol) was dissolved in dichloromethane (2 mL) and to that solution, trifluoroacetic acid (1 mL) was added. The resulting solution was stirred at room temperature for 1 h. The reaction was deemed complete by TLC (4:1 v/v hexanes/EtOAc). The volatiles were removed in vacuo. The residue was then dissolved in DMF (1 mL) and treated with diisopropylethyl amine (140 μL, 0.80 mmol). This solution was set aside. In a separate flask, a solution containing the dipeptide (R)-2-(3-tert-butoxycarbonylamino-propionylamino)-3-(2,4-dichloro-phenyl)-propionic acid (178 mg, 0.44 mmol), diisopropylethyl amine (140 μL, 0.80 mmol) in DMF (2 mL), was treated with HBTU (200 mg, 0.52 mmol). The resulting golden yellow solution was stirred at room temperature, under N₂, for 30 minutes. The solution containing the deprotected amine was added to this, and the resulting mixture was stirred for 16 h at room temperature. The reaction was diluted with EtOAc (30 mL) and washed with 0.1 N HCl and then with saturated NaHCO₃. The organics were washed with brine, dried over anhydrous MgSO₄ and filtered. Evaporation gave a residue that was dissolved in dichloromethane (4 mL) and treated with trifluoroacetic acid (2 mL). After 2 h, the volatiles were removed under vacuum and the residue was purified by preparative HPLC/MS to give Example 27. Yield=88 mg (0.17 mmol, 42%). LCMS m/z 527 (M⁺+1).

### Example 28

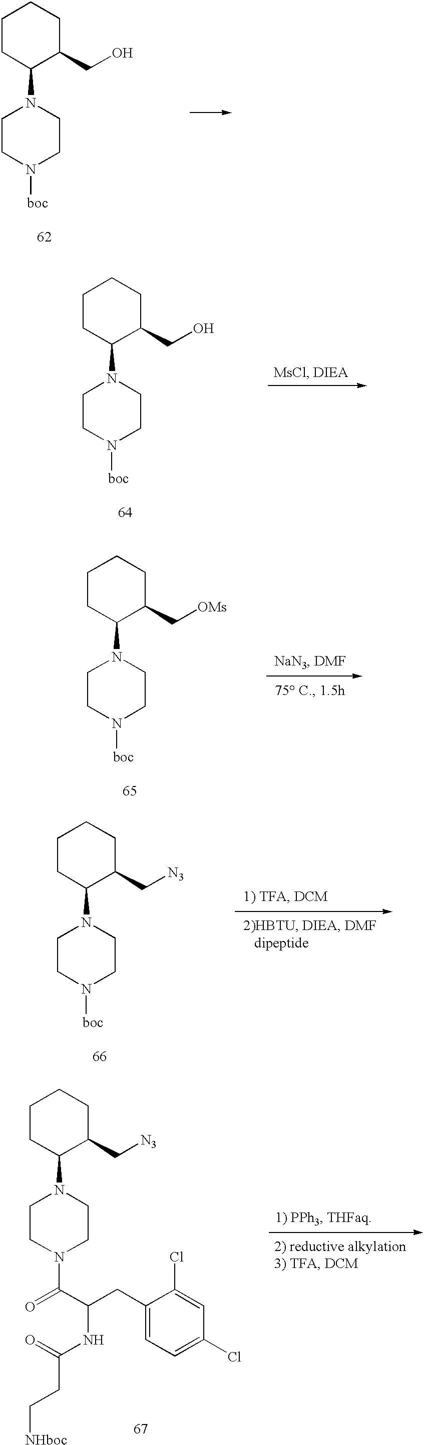

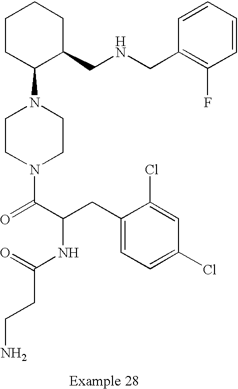

Step 28A: cis-4-(2-hydroxymethyl-cyclohexyl)-piperazine-1-carboxylic Acid Tert-Butyl Ester

cis-4-(2-Ethoxycarbonyl-cyclohexyl)-piperazine-1-carboxylic acid tert-butyl ester 62 (3.40 g, 10.0 mmol) was dissolved in THF (25 mL) and added slowly to a stirred suspension of LiAlH₄ (0.80 g, 20.0 mmol) in THF (50 mL), at 0° C. under N₂. The resulting mixture was stirred at 0° C. for 30 min. and then at room temperature for 1 h. The reaction mixture was cooled to 0° C., and quenched carefully by the addition of EtOAc (˜5 mL), followed by saturated Rochelle's salt solution (˜50 mL). EtOAc (100 mL) was added and the resulting white suspension was stirred vigorously for 30 min. The layers were separated and the organics were washed with brine, dried over anhydrous MgSO₄ and filtered. Evaporation gave the compound 64 as an oil, which solidified upon standing. Yield=2.40 g (8.1 mmol, 81%). LCMS m/z 299 (M⁺+1).

Step 28B: cis-4-(2-Methanesulfonyloxymethyl-cyclohexyl)-piperazine-1-carboxylic Acid Tert-Butyl Ester

Methanesulfonyl chloride (373 μL, 4.8 mmol) was added dropwise to a stirring solution of cis-4-(2-hydroxymethyl-cyclohexyl)-piperazine-1-carboxylic acid tert-butyl ester 64 (1.19 g, 4.0 mmol) and diisopropylethyl amine (1.40 mL, 8.0 mmol) in THF (20 mL), at 0° C. under N₂. The mixture was stirred at 0° C. for 30 minutes, and then allowed to reach room temperature. After 1 h, the reaction was diluted with EtOAc (100 mL) and washed with H₂O, diluted HCl and brine. The organics were dried over MgSO₄ and filtered. Evaporation gave the compound 65 as a thick yellow oil (780 mg, 2.1 mmol, 52%), which was used without any further purification. LCMS m/z 377 (M⁺+1).

Step 28C: cis-4-(2-Azidomethyl-cyclohexyl)-piperazine-1-carboxylic Acid Tert-Butyl Ester

A solution of cis-4-(2-methanesulfonyloxymethyl-cyclohexyl)-piperazine-1-carboxylic acid tert-butyl ester 65 (780 mg, 2.1 mmol) and sodium azide (650 mg, 10.0 mmol) in DMF (10 mL) was heated to 75° C. for 1.5 h. The reaction was deemed complete by LCMS. It was then cooled, diluted with EtOAc (100 mL), washed with H₂O, 0.1N HCl, and brine. The organics were dried over MgSO4 and filtered. Evaporation gave the 66 as a yellow oil, which was used without any further purification. Yield=743 mg (>100%). LCMS m/z 324 (M⁺+1).

Step 28D: {2-[2-[4-cis-(2-Azidomethyl-cyclohexyl)-piperazin-1-yl-]-(R)-(2,4-dichloro-benzyl)-2-oxo-ethylcarbamoyl]-ethyl}carbamic Acid Tert-Butyl Ester

cis-4-(2-Azidomethyl-cyclohexyl)-piperazine-1-carboxylic acid tert-butyl ester 66 (669 mg, 2.1 mmol) was dissolved in dichloromethane (10 mL) and treated with trifluoroacetic acid (5 mL). The resulting solution was stirred at room temperature for 4.5 h. The volatiles were then removed in vacuo and the residue was dissolved in DMF (5 mL) and treated with diisopropylethyl amine (720 μL, 4.1 mmol). This solution was set aside. In a separate flask, a solution containing the dipeptide (R)-2-(3-tert-butoxycarbonylamino-propionylamino)-3-(2,4-dichloro-phenyl)-propionic acid (920 mg, 2.3 mmol), diisopropylethyl amine (720 μL, 4.1 mmol) in DMF (11 mL), was treated with HBTU (1.02 g, 2.7 mmol). The resulting golden yellow solution was stirred at room temperature, under N₂, for 30 minutes. The solution containing the deprotected amine was added to this, and the resulting mixture was stirred for 66 h at room temperature. The reaction was diluted with EtOAc (100 mL) and washed with 0.1 N HCl and then with saturated NaHCO₃. The organics were washed with brine, dried over anhydrous MgSO₄ and filtered. Evaporation gave a residue that was purified by silica-gel chromatography, eluting with 3:2 v/v mixture of hexanes and EtOAc, respectively. Compound 67 was obtained as a tan foam. Yield=475 mg (0.8 mmol,38%). LCMS m/z 610 (M⁺+1).

Step 28E: 3-Amino-N-[1-(R)-(2,4-dichloro-benzyl)-2-(4-cis{2-[(2-fluoro-benzylamino)-methyl]-cyclohexyl}-piperazin-1-yl)-2-oxo-ethyl]-propionamide

Triphenylphosphine (245 mg, 0.94 mmol) was added to a stirring solution of {2-[2-[4-cis-(2-azidomethyl-cyclohexyl)-piperazin-1-yl]-1-(R)-(2,4-dichloro-benzyl)-2-oxo-ethylcarbamoyl]-ethyl}-carbamic acid tert-butyl ester 67 (475 mg, 0.78 mmol) in THF (8 mL) and H₂O (1 mL). The mixture was stirred at room temperature, and it was monitored by LCMS. After 24 h, the volatiles were removed under vacuum and the residue was purified by preparative HPLC/MS. The pure amine (15 mg, 0.03 mmol) was dissolved in MeOH (1 mL) and treated with 2-fluorobenzaldehyde (2 drops). The resulting solution was stirred at room temperature for 1 h. NaBH₄ (30 mg) was added in one portion, followed by gas evolution. The reaction mixture was then diluted with EtOAc (20 mL), washed with H₂O and brine. The organics were dried over anhydrous MgSO₄, filtered and concentrated under vacuum. The residue was dissolved in a 1:1 v/v mixture of dichloromethane and trifluoroacetic acid (2 mL) and stirred for 1 h. The volatiles were removed in vacuo and Example 28 was obtained after purification by preparative HPLC/MS. Yield=2.1 mg (3.6 μmol, 19%). LCMS m/z 592 (M⁺+1).

### Example 29

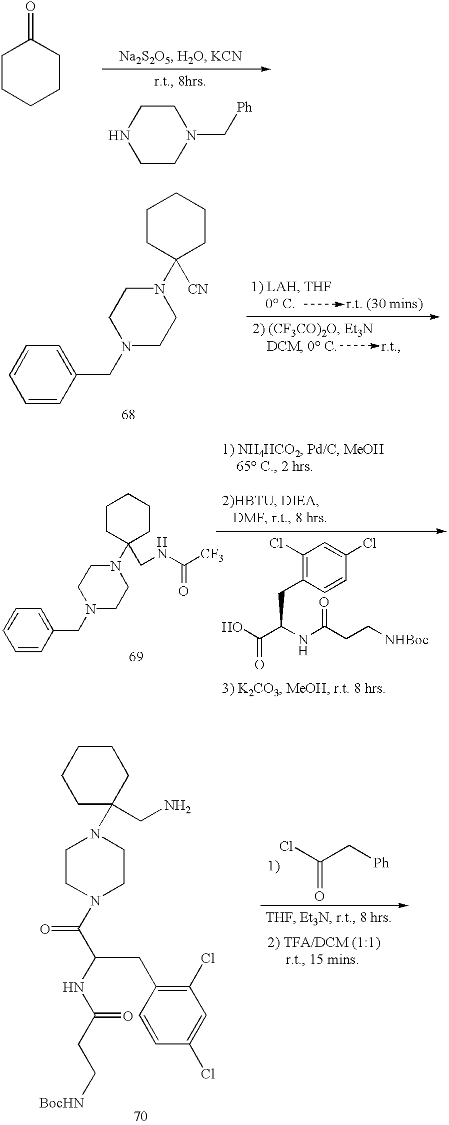

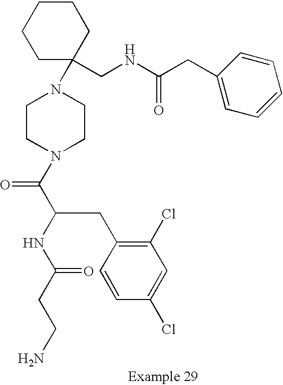

Step 29A: 1-(1-Cyanocyclohexyl)-4-benzylpiperazine 68:

Cyclohexanone (7.3 mL, 70 mmol) was dissolved in water (140 mL) along with Na₂S₂O₅ (6.4 g, 35 mmol). The mixture was allowed to stir at room temperature for 1.5 hours then 1-benzylpiperazine (12.2 mL, 70 mmol) was added. The mixture was stirred for 2 hours and KCN (4.8 g, 74 mmol) was added to the reaction mix. The reaction mixture was then allowed to stir at room temperature overnight. The product was then extracted with dichloromethane (3×200 mL). The combined extracts were dried over anhydrous MgSO₄, filtered, and solvent was removed under vacuum. Compound 68 was recovered as a white solid in quantitative yield.

Step 29B: 1-[1-(Trifluoroacetamidomethyl)cyclohexyl]-4-benzylpiperazine 69:

1-(1-Cyanocyclohexyl)-4-benzylpiperazine 68 (10 g, 35.3 mmol) was dissolved in ether (176 mL) and added dropwise to a mixture of LiAlH₄ (2.7 g, 71 mmol) in ether (353 mL) at room temperature. After the addition, the mixture was allowed to stir at room temperature for 0.5 hours. The reaction was then quenched by adding 2 mL H₂O, followed by 1.5 mL 20% NaOH, then 7 mL 11₂O. The reaction mixture was then filtered through celite and the residue was washed with ether. The ethereal mother liquor was dried over anhydrous MgSO₄ and solvent was removed under vacuum. The intermediate amine product was recovered in 94% yield without any further purification. This amine intermediate (9.5 g, 33 mmol) was then dissolved in dichloromethane (100 ml,) along with Et₃N (4.8 mL, 34.7 mmol) and the reaction mixture was cooled to 0° C. To the reaction flask, trifluoroacetic anhydride (4.9 ml, 34.7 mmol) was added and the reaction was stirred at 0° C. for 10 minutes then at room temperature for 4 hours. Compound 69 was recovered as a clear oil (quantitative yield) after the reaction mixture was concentrated under vacuum. No further purification was needed.

Step 29C: 3-Boc-amino-N-[1-(2,4-dichlorobenzyl)-2-oxo-2-(4-{2-[(2-amino)methyl]cyclohexyl}piperazin-1-yl)ethyl]propionamide 70

1-[1-(Trifluoroacetamidomethyl)cyclohexyl]-4-benzylpiperazine69 (13 g, 33 mmol) was dissolved in MeOH (192 mL) and the solution was degassed with nitrogen for 5 minutes. To the reaction flask, 10% by weight Pd on carbon (5 g) was added along with ammonium formate (6.2 g, 99 mmol). The reaction was allowed to stir at 65° C. for 2 hours. The reaction was then cooled to room temperature, filtered through celite, washed with degassed methanol, and solvent was removed under vacuum. The resulting residue was dissolved in dichloromethane (150 mL) and washed with sat. NaHCO₃ (3×150 mL) followed by washing with sat. NaCl solution (1×200 mL). The organic layer was then dried over anhydrous MgSO₄, filtered, and solvent was removed under vacuum. The deprotected piperazine was recovered as a clear oil in 86% yield without further purification. This deprotected piperazine intermediate (2.93 g, 10 mmol) was then added to a solution of dipeptide (R)-2-(3-tert-butoxycarbonylamino-propionylamino)-3-(2,4-dichlorophenyl)-propionic acid (4 g, 9.87 mmol) that had been previously stirred for 1 hour at room temperature in DMF (42 mL) with HBTU (3.7 g, 9.87 mmol) and diisopropylethylamine (3.4 mL, 19.7 mmol). The reaction mixture was then allowed to stir for an additional 8 hours at room temperture. The reaction was then diluted with ethyl acetate (200 mL) and washed with washed with sat. NaHCO₃ (3×150 mL) followed by washing with sat. NaCl solution (1×200 ml). The organic layer was then dried over anhydrous Na₂SO₄, filtered, and solvent was removed under vacuum. The residue was purified by column chromatography on silica using 60% ethyl acetate/hexanes as the eluent (Rf=0.3). The cyclohexyl piperazine peptide product was recovered as a clear oil in 54% yield (3.65 g, 5.4 mmol). This cyclohexyl piperazine peptide intermediate (2.4 g, 3.5 mmol) was then dissolved in a MeOH (50 mL)/H₂O (4 mL) mixture along with K₂CO₃ (11.8 g) and the reaction was allowed to stir at 65° C. for 8 hours. The reaction was then cooled to room temperature and the reaction mixture was diluted with dichloromethane (150 mL). The reaction mixture was then washed with H₂O (3×100 mL) followed by washing with sat. NaCl solution (1×150 mL). The organic layer was then dried over anhydrous MgSO₄, filtered, and solvent was removed under vacuum. Compound 70 was recovered as a clear yellow oil in 86% yield without any further purification needed.

Step 29D: 3-Amino-N-[1-(2,4-dichlorobenzyl)-2-oxo-2-(4-{2-[(2-phenylacetamido)methyl]cyclohexyl}piperazin-1-yl)ethyl]propionamide

In a 4 mL reaction vial, a 1 mL aliquot of a 0.1M aminomethyl cyclohexyl peptide 70 THF stock solution was added along with Et₃N (14 uL, 0.1 mmol). To the reaction vial, phenylacetyl chloride (13.2 uL, 0.1 mmol) was added and the reaction was allowed to stir at room temperature for 8 hours. The solvent was then removed by evaporation under a stream on nitrogen and the residue was dissolved in 2 mL of dichloromethane/TFA (1:1). The reaction mixture was allowed to stir at room temperature for 15 minutes then evaporated to dryness. The residue was then dissolved in 1 mL of methanol and the crude product was purified by preparative HPLC. Example 29 was recovered as the TFA salt in 9% overall yield. MS: calc. for C₃₁H₄₁Cl₂N₅O₃: 601.26; Found: 602.1 (M+H); retention time: 1.938 minutes; Method info: APCI positive ion scan 100-1000 Frag V=80; 100% 0.05%TFA/H₂O to 90% ACN/0.05%TFA over 2 min, 2.5 min run, ODS-AQ column.

By the general procedures set forth above, the following compounds were also made.

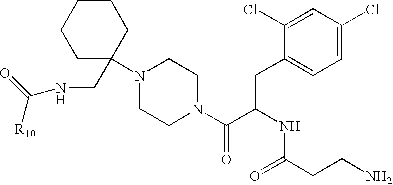

| Example | —R₁₀ | MS(MH⁺) | MW |
|---|---|---|---|
| 29-1 | Ph—CH₂— | 602 | 602.6 |
| 29-2 | —CF₃ | 580 | 580.5 |
| 29-3 | 4-F—Ph—CH₂— | 620 | 620.6 |
| 29-4 | 4-Cl—Ph—CH₂— | 637 | 637.0 |
| 29-5 | 3-OMe—Ph—CH₂— | 632 | 632.6 |
| 29-6 | 4-OMe—Ph—CH₂— | 632 | 632.6 |
| 29-7 | 3,4-di-OMe—Ph—CH₂— | 662 | 662.7 |
| 29-8 | 2-Thiophene-CH₂— | 608 | 608.6 |

### Example 30

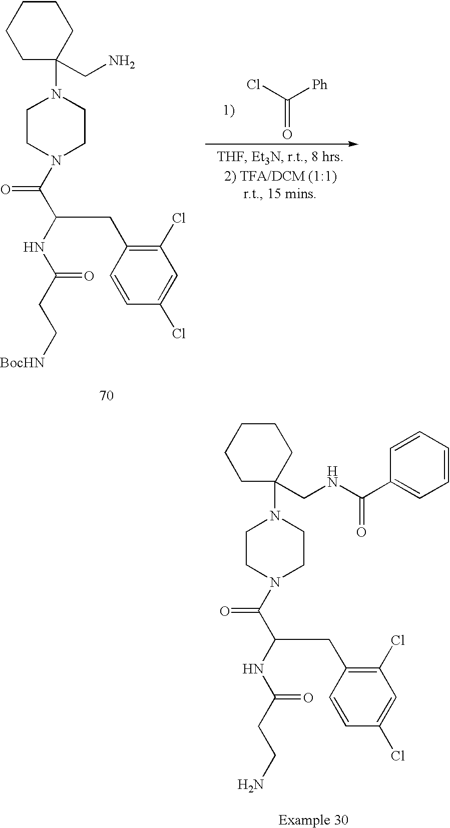

Step 30A: 3-Amino-N-[1-(2,4-dichlorobenzyl)-2-oxo-2-(4-{2-[(2-benzoylamino)methyl]cyclohexyl}piperazin-1-yl)ethyl]propionamide

In a 4 mL reaction vial, a 1 mL aliquot of the 0.1 M aminomethyl cyclohexyl peptide 70 THF stock solution was added along with Et₃N (14 uL, 0.1 mmol). To the reaction vial, benzoyl chloride (11.6 uL, 0.1 mmol) was added and the reaction was allowed to stir at room temperature for 8 hours. The solvent was then removed by evaporation under a stream of nitrogen and the residue was dissolved in 2 mL of dichloromethane/TFA (1:1). The reaction mixture was allowed to stir at room temperature for 15 minutes then evaporated to dryness. The residue was then dissolved in 1 mL of methanol and the crude product was purified by preparative HPLC. Example 30 was recovered as the TFA salt in 54% overall yield. MS: calc. for C₃₀H₃₉Cl₂N₅O₃: 587.24; Found: 588.1 (M+H); retention time: 1.907 minutes; Method info: APCI positive ion scan 100-1000 Frag V=80; 100% 0.05%TFA/H₂O to 90% ACN/0.05%TFA over 2 min, 2.5 min run, ODS-AQ column.

By the general procedures set forth above, the following compounds were also made.

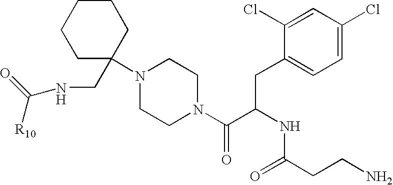

| Example | C(O)R₁₀ | MS(MH+) | MW |
|---|---|---|---|
| 30-1 | benzoyl | 588 | 588.6 |
| 30-2 | 4-methylbenzoyl | 602 | 602.6 |
| 30-3 | 4-tert-butylbenzoyl | 644 | 644.7 |
| 30-4 | 4-fluorobenzoyl | 606 | 606.6 |
| 30-5 | 4-chlorobenzoyl | 623 | 623.0 |
| 30-6 | 4-bromobenzoyl | 667 | 667.5 |
| 30-7 | 4-methoxybenzoyl | 618 | 618.6 |
| 30-8 | 4-trifluoromethylbenzoyl | 656 | 656.6 |
| 30-9 | 4-trifluoromethoxybenzoyl | 672 | 672.6 |
| 30-10 | 4-nitrobenzoyl | 633 | 633.6 |
| 30-11 | 2-methoxybenzoyl | 618 | 618.6 |
| 30-12 | 2-furancarbonyl | 578 | 578.5 |
| 30-13 | 2-thiophenecarbonyl | 594 | 594.6 |
| 30-14 | 3-pyridylcarbonyl | 589 | 589.6 |
| 30-15 | 4-pyridylcarbonyl | 589 | 589.6 |

### Example 31

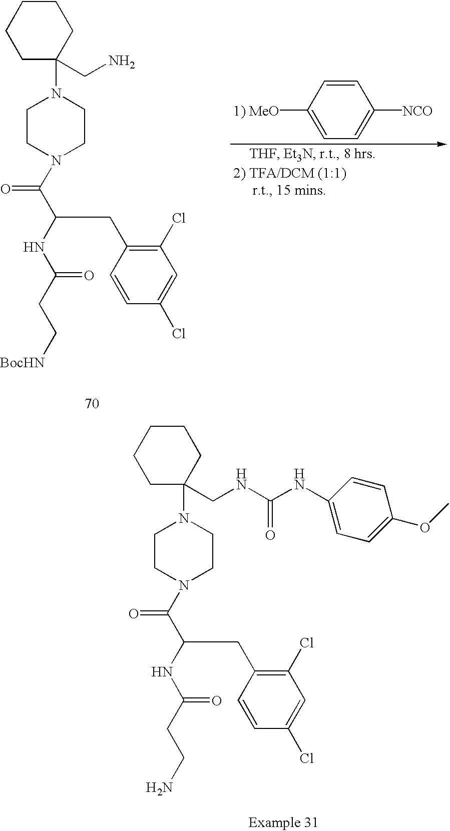

Step 31A:

In a 4 mL reaction vial, a 1 mL aliquot of the 0.1 M aminomethyl cyclohexyl peptide 70 THF stock solution was added along with Et₃N (14 uL, 0.1 mmol). To the reaction vial, 4-methoxyphenyl isocyanate (13 uL, 0.1 mmol) was added and the reaction was allowed to stir at room temperature for 8 hours. The solvent was then removed by evaporation under a stream on nitrogen and the residue was dissolved in 2 mL of dichloromethane/TFA (1:1). The reaction mixture was allowed to stir at room temperature for 15 minutes then evaporated to dryness. The residue was then dissolved in 1 mL of methanol and the crude product was purified by preparative HPLC. Example 31 was recovered as the TFA salt in 46% overall yield. MS: calc. for C₃₁H₄₂Cl₂N₆O₄: 632.26; Found: 633.1 (M+H); retention time: 1.925 minutes; Method info: APCI positive ion scan 100-1000 Frag V=80; 100% 0.05%TFA/H₂O to 90% ACN/0.05%TFA over 2 min, 2.5 min run, ODS-AQ column.

By the general procedures set forth above, the following compounds were also made.

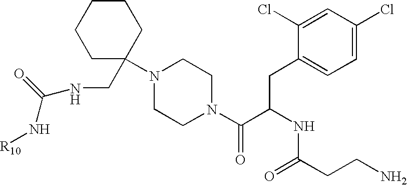

| Example | R₁₀ | MS(MH+) | MW |
|---|---|---|---|
| 31-1 | 4-methoxyphenyl | 633 | 633.6 |
| 31-2 | 4-fluorophenyl | 621 | 621.6 |
| 31-3 | 4-chlorophenyl | 638 | 638.0 |
| 31-4 | 4-nitrophenyl | 648 | 648.6 |
| 31-5 | 4-dimethylaminophenyl | 646 | 646.7 |
| 31-6 | 4-methoxycarbonylphenyl | 661 | 661.6 |

### Example 32

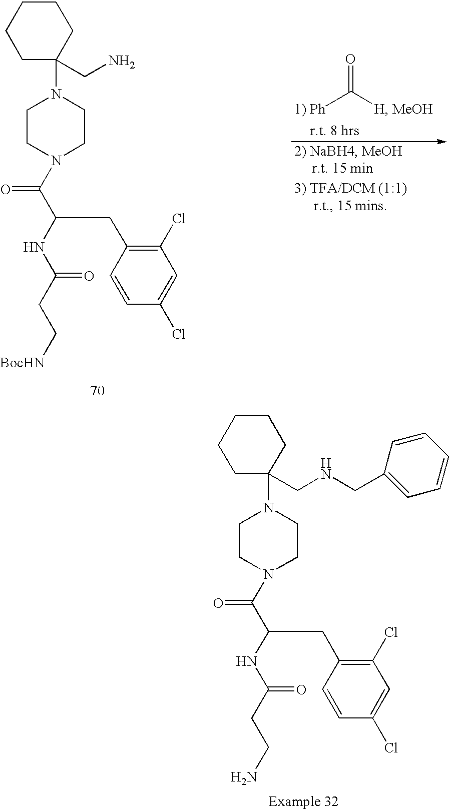

Step 32A: 3-Amino-N-[1-(2,4-dichlorobenzyl)-2-oxo-2-(4-{2-[(2-benzlamino)methyl]cyclohexyl}piperazin-1-yl)ethyl]propionamide

In a 4 mL reaction vial, a 1 mL aliquot of the 0.1 M aminomethyl cyclohexyl peptide 70 MeOH stock solution was added along with benzaldehyde (10 uL, 0.1 mmol). The reaction was allowed to stir at room temperature for 8 hours. Then, to the reaction vial, NaBH₄ (6.1 mg, 0.16 mmol) was added and the reaction was allowed to stir at room temperature for an additional 15 minutes. The reaction was then quenched with 1 mL of 1N NaOH and the product was extracted with ether. The ethereal extract was then concentrated under a stream on nitrogen and the residue was dissolved in 2 mL of dichloromethane/TFA (1:1). The reaction mixture was allowed to stir at room temperature for 15 minutes then evaporated to dryness. The residue was then dissolved in 1 mL of methanol and was purified by preparative HPLC. Example 32 was recovered as the TFA salt in 52% overall yield. MS: calc. for C₃₀H₄₁C₁₂N₅O₂: 573.26; Found: 574.1 (M+H); retention time: 1.984 minutes; Method info: APCI positive ion scan 100-1000 Frag V=80; 100% 0.05%TFA/H₂O to 90% ACN/0.05%TFA over 2 min, 2.5 min run, ODS-AQ column.

By the general procedures set forth above, the following compounds were also made.

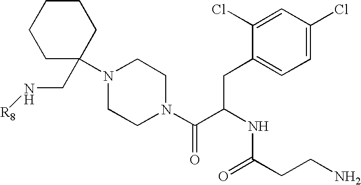

| Example | R₈ | MS(MH+) | MW |
|---|---|---|---|
| 32-1 | benzyl | 574 | 547.6 |
| 32-2 | hydrogen | 484 | 484.5 |
| 32-3 | 2-fluorobenzyl | 592 | 592.6 |
| 32-4 | 4-cyanobenzyl | 599 | 599.6 |
| 32-5 | 4-fluorobenzyl | 592 | 592.6 |
| 32-6 | 4-trifluorobenzyl | 642 | 642.6 |
| 32-7 | 4-trifluoromethoxylbenzyl | 658 | 658.6 |
| 32-8 | 4-dimethylaminobenzyl | 617 | 617.7 |
| 32-9 | 1-thizolemethyl | 581 | 581.6 |
| 32-10 | thiophenylmethyl | 580 | 580.6 |
| 32-11 | 2-pyridylmethyl | 575 | 575.6 |
| 32-12 | phenethyl | 588 | 588.6 |
| 32-13 | 3-phenylpropyl | 602 | 602.6 |
| 32-14 | isobutyl | 540 | 540.6 |
| 32-15 | 3,3-dimethylbutyl | 568 | 568.6 |
| 32-16 | cyclohexylmethyl | 580 | 580.6 |

### Example 33

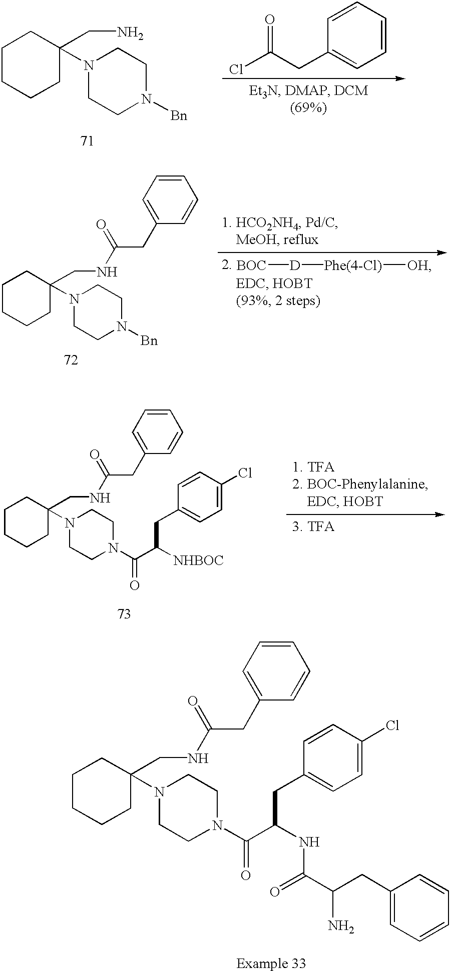

Step 33A: 1-[1-(Phenylacetamidomethyl)cyclohexyl]-4-benzylpiperazine

To a stirring solution of 1-[1-(aminomethyl)cyclohexyl]-4-benzylpiperazine 71 (9.29 g, 32.4 mmol, made according to steps 29A and 29B) and triethylamine (8.2 g, 81 mmol) in dry dichloromethane (80 mL) at 0° C. under nitrogen was added phenylacetyl chloride (5.5 g, 36 mmol). After warming to RT and stirring 3 h, DMAP (0.1 0g, 0.82 mmol) and additional phenylacetyl chloride (2.6 g, 17 mmol) were added, and stirring was continued for 1 h. The mixture was then diluted with dichloromethane (100 mL) and washed with sat. aq. sodium bicarbonate (100 mL) and brine (100 mL). The organic layer was dried (magnesium sulfate), concentrated and purified by column chromatography (70:30 dichloromethane: ethyl acetate to 96:4 dichloromethane:methanol) to give the amide 72 as a yellow solid (9.0 g, 69%). MS=406.1 ((M+H)⁺).

Step 33B: 1-[1-(tert-Butoxycarbonylamido)-2-(2,4-dichlorophenyl)propionyl]-4-{2-[(phenylacetamido)methyl]cyclohexyl}piperazine

To 1-[1-(phenylacetamidomethyl)cyclohexyl]-4-benzylpiperazine 72 (4.0 g, 9.9 mmol) in dry, degassed methanol (70 mL) was added ammonium formate (1.9 g, 30 mmol), followed by 10% Pd/C (2.0 g, 1.9 mmol). The mixture was refluxed under nitrogen for 40 min, then cooled and filtered over celite. Concentration of the filtrate gave the crude free amine as a yellow oil (3.1 g, 100%). MS=316.1 ((M+H)⁺).

A portion of the crude amine (2.23 g, 7.08 mmol) was immediately dissolved in dichloromethane (100 mL). BOC-D-Phe(4-Cl)—OH (2.23 g, 7.40 mmol), followed by HOBT (1.00 g, 7.40 mmol) were added and the mixture was stirred for 10 min. EDC (1.42 g, 7.40 mol) was then added, and the mixture was stirred overnight. The solution was diluted with dichloromethane (100 mL) and washed with sat. aq. sodium bicarbonate (2×100 mL), dried (magnesium sulfate), concentrated and purified by column chromatography (96:4 dichloromethane: methanol) to give the compound 73 as an orange foam (3.92 g, 93%). MS 597.2 ((M+H)⁺).

Step 33C: 1-[1-(Acetamido)-2-(2,4-dichlorophenyl)propionyl]-4-{2-[(phenylacetamido)methyl]cyclohexyl}piperazine

A sample of 1-[1-(tert-Butoxycarbonylamido)-2-(2,4-dichlorophenyl)propionyl]-4-{2-[(phenylacetamido)methyl]cyclohexyl}piperazine3 (2.0 g, 3.4 mmol) was dissolved in dichloromethane (10 mL), and TFA (10 mL) was added. The solution was stirred for 20 min, then evaporated, re-dissolved in dichloromethane (50 mL), and washed with sat. aq. sodium bicarbonate/sodium carbonate solution (pH 9, 25 mL). The aqueous layer was extracted with dichloromethane (50 mL), and the combined organics were washed with brine (25 mL), dried (magnesium sulfate) and concentrated to give the crude free base (1.6 g, 100%). To the free base (40 mg, 0.081 mmol) in dichloromethane (0.5 mL) was added HOBT (11 mg, 0.081 mmol) and the N-BOC-phenylalanine(21.5 mg,0.081 mmol). The mixture was stirred for 10 min, then a solution of EDC (16 mg, 0.081 mmol) in dichloromethane (0.5 mL) was added. The mixture was stirred overnight, then washed with sat. aq. sodium bicarbonate (0.5 mL), dried (magnesium sulfate) and concentrated. Dichloromethane (1 mL) and TFA (1 mL) were added and the mixture was stirred for 30 min., concentrated and purified by preparative LCMS to give Example 33. (MH⁺=644)

By the general procedures set forth above, the following compounds were also made.

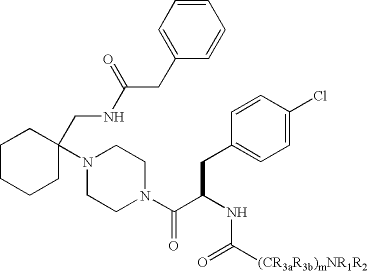

| Example | —(CR₃ₐR_{3b})ₘNR₁R₂ | MS(MH+) | MW |
|---|---|---|---|
| 33-1 | 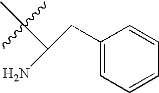 | 644 | 644.3 |
| 33-2 | 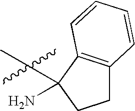 | 656 | 656.3 |
| 33-3 | 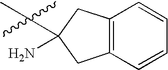 | 656. | 656.3 |
| 33-4 | 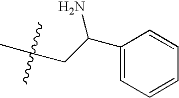 | 644 | 644.3 |
| 33-5 | 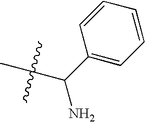 | 630 | 630.2 |
| 33-6 | 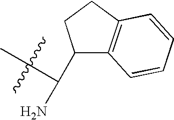 | 670 | 670.3 |
| 33-7 | 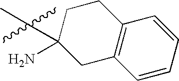 | 670 | 670.3 |
| 33-8 | 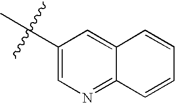 | 652 | 652.2 |
| 33-9 | 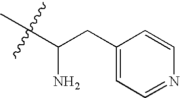 | 645 | 645.2 |
| 33-10 | 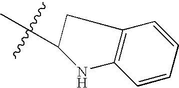 | 642 | 642.2 |
| 33-11 | 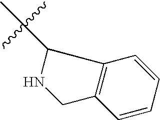 | 642 | 642.2 |
| 33-12 | 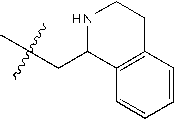 | 670 | 670.3 |
| 33-13 | 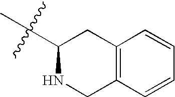 | 656 | 656.3 |
| 33-14 | 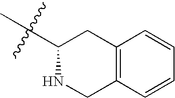 | 656 | 656.3 |

It will be appreciated that, although specific embodiments of the invention have been described herein for purposes of illustration, various modifications may be made without departing from the spirit and scope of the invention. Accordingly, the invention is not limited except as by the appended claims.

1. A compound having the following structure: 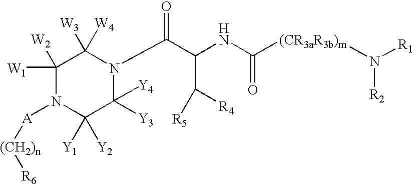
or a stereoisomer, prodrug or pharmaceutically acceptable salt thereof,
wherein:
n is 0, 1, 2, or 3;
m is 1, 2, 3, or 4;
A is alkanediyl optionally substituted with R₇;
R₁ and R₂ are the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl, or —C(═O)R₁₀;
or R₁ and R₂ taken together with the nitrogen atom to which they are attached form heterocycle or substituted heterocycle;
R₃ₐ and R_{3b} are the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
or R₃ₐ and R_{3b} taken together with the carbon atom to which they are attached form a homocycle, substituted homocycle, heterocycle, or substituted heterocycle;
or R₃ₐ and the carbon atom to which it is attached taken together with one or both of R₁ and R₂ and the nitrogen to which it is attached form heterocycle or substituted heterocycle;
R₄ is aryl, substituted aryl, heteroaryl, or substituted heteroaryl;
R₅ is hydrogen, hydroxy, alkyl, substituted alkyl, aryl, substituted aryl, heterocycle, or substituted heterocycle;
R₆ is cyano, nitro, heterocycle, substituted heterocycle, —NR₈R₉, —C(═O)NR₈R₉, —C(═O)OR₈, —OC(═O)OR₈, —OC(═O)R₈, —OC(═O)NR₈R₉, —NR₈C(═O)OR₈, —NR₈C(═O)R₁₀, —NR₈C(═O)NR₈R₉, —NR₈S(═O)ₚR₁, —S(═O)ₚR₁, —S(═O)ₚNR₈R₉, —NR₈S(═O)ₚNR₈R₉, or —OR₁₂;
R₇ is alkyl, substituted alkyl, aryl, substituted aryl, arylalkyl, substituted arylalkyl, heterocycle, substituted heterocycle, heterocyclealkyl, substituted heterocyclealkyl, cyano, nitro, —NR₈R₉, —C(═O)NR₈R₉, —C(═O)OR₈, —NR₈C(═O)R₁₀, —NR₈C(═O)NR₈R₉, —NR₈S(═O)ₚR₁₁, —S(═O)ₚR₁₁, —NR₈S(═O)ₚNR₈R₉, or —OR₁₂;
R₈ and R₉ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₁₀, R₁₁, and R₁₂ are the same or different and, at each occurrence, independently hydrogen, halogen, cyano, alkyl, substituted alkyl, aryl, substituted aryl, arylalkyl, substituted arylalkyl, heterocycle, substituted heterocycle, heterocyclealkyl or substituted heterocyclealkyl;
W₁, W₂, W₃, W₄, Y₁, Y₂, Y₃ and Y₄ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, cyano, nitro, —NR₈R₉, —C(═O)NR₈R₉, —C(═O)OR₁₀, —NR₈C(═O)R₁₀, —NR₈C(═O)NR₈R₉, —NR₈S(═O)ₚR₁₁, —S(═O)ₚR₁₁, —NR₈S(═O)ₚNR₈R₉, or —OR₁₂;
or any of one of W₁, W₂, W₃ or W₄ and the carbon to which it is attached together with any one of Y₁, Y₂, Y₃ or Y₄ and the carbon to which it is attached form a bridging heterocycle or substituted heterocycle; and
p is, at each occurrence, 0, 1 or 2.

2. The compound of claim 1 wherein A is cyclic alkyl.

3. The compound of claim 2 wherein A is cyclohexyl or cycloheptyl.

4. The compound of claim 1 wherein A is lower alkyl.

5. The compound of claim 1 where R₁ and R₂ are the same or different and independently hydrogen or lower alkyl.

6. The compound of claim 1 where R₃ₐ and R_{3b} are the same or different and independently hydrogen or lower alkyl.

7. The compound of claim 1 wherein R₃ₐ and the carbon atom to which it is attached taken together with R₁ and the nitrogen to which it is attached form heterocycle or substituted heterocycle.

8. The compound of claim 1 wherein R₄ is substituted aryl.

9. The compound of claim 1 wherein R₅ is hydrogen.

10. The compound of claim 1 wherein R₆ is heterocycle, substituted heterocycle, —NR₈R₉, —C(═O)NR₈R₉, —C(═O)OR₈, —OC(═O)OR₈, —OC(═O)R₈, —OC(═O)NR₈R₉, —NR₈C(═O)OR₈, —NR₈C(═O)R₁₀, —NR₈C(═O)NR₈R₉, —NR₈S(═O)ₚR₁₁, —S(═O)ₚR₁₁, —S(═O)ₚNR₈R₉, —NS(═O)ₚNR₈R₉, or —OR₁₂.

11. The compound of claim 10 where R₆ is tetrazolyl, triazolyl, —C(═O)OR₈, —NR₈C(═O)R₁₀, —C(═O)NR₈R₉ or —NR₈S(═O)ₚR₁₁.

12. The compound of claim 1 wherein n is 1.

13. A pharmaceutical composition comprising a compound of claim 1 in combination with a pharmaceutically acceptable carrier.

14. A method for altering a disorder associated with the activity of a melanocortin receptor, comprising administering to a patient in need thereof an effective amount of a compound of claim 1.

15. The method of claim 14 wherein the melanocortin receptor is melanocortin 3 receptor.

16. The method of claim 14 where the melanocortin receptor is melanocortin 4 receptor.

17. The method of claim 14 wherein the compound is an antagonist of the melanocortin receptor.

18. The method of claim 14 wherein the compound is an antagonist of the melanocortin receptor.

19. The method of claim 14 wherein the disorder is an eating disorder.

20. The method of claim 19 wherein the eating disorder is cachexia.

21. The method of claim 14 wherein the disorder is a sexual disfunction.

22. The method of claim 21 where the sexual disfunction is erectile disfunction.

23. The method of claim 14 wherein the disorder is a skin disorder.

24. The method of claim 14 where the disorder is chronic pain.

25. The method of claim 14 where the disorder is anxiety or depression.

26. The method of claim 14 wherein the disorder is obesity.
